(19) United States
(12) Patent Application Publication
(10) Pub. No.: US 2009/0325924 A1
(43) Pub. Date: Dec. 31, 2009

(54) GPCR Agonists

(76) Inventors: Stuart Edward, Oxford (GB); Graham John Dawson, Oxford (GB); Matthew Colin Thor Fyfe Oxford, Oxford (GB); Lisa Sarah Bertram, Oxford (GB); William Gattrell, Oxford (GB); Revathy Perpetua Jeevaratnam, Oxford (GB); John Keily, Oxford (GB); Neela Sumit Mistry, Oxford (GB); Martin James Procter, Oxford (GB); Chrystelle Marie Rasamison, Oxford (GB); Philip John Rushworth, Oxford (GB); Colin Peter Sambrook-Smith, Oxford (GB); David French Stonehouse, Oxford (GB)

Correspondence Address: OSI PHARMACEUTICALS, INC., 41 PINELAWN ROAD, MELVILLE, NY, 11747, US

(21) Appl. No.: 11/922,765

(22) PCT Filed: Jun. 29, 2006
(86) PCT No.: PCT/GB2006/050176

§ 371 Date: Mar. 23, 2009

(30) Foreign Application Priority Data
Jun. 30, 2005 (GB) 0513257.6
Mar. 20, 2006 (GB) 0605539.6

(51) Int. Cl. A61K 31/4245; C07D 413/14; C07D 413/12; C07D 271/06; A61K 31/454; A61K 31/5377; A61K 31/496; A61K 31/506; A61P 3/00

(52) U.S. Cl. 514/210.18; 546/210; 548/131; 544/130; 544/364; 544/331; 514/326; 514/364; 514/236.2; 514/253.1; 514/275

(57) ABSTRACT

Compounds of formula (I): or pharmaceutically acceptable salts thereof, are GPCR agonists and are useful as for the treatment of obesity and diabetes.

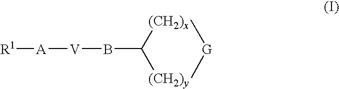

### BACKGROUND OF THE INVENTION

The present invention is directed to G-protein coupled receptor (GPCR) agonists. In particular, the present invention is directed to GPCR agonists that are useful for the treatment of obesity, e.g. as regulators of satiety, and for the treatment of diabetes.

Obesity is characterized by an excessive adipose tissue mass relative to body size. Clinically, body fat mass is estimated by the body mass index (BMI; weight (kg)/height (m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

Pharmacological approaches to the treatment of obesity have been mainly concerned with reducing fat mass by altering the balance between energy intake and expenditure. Many studies have clearly established the link between adiposity and the brain circuitry involved in the regulation of energy homeostasis. Direct and indirect evidence suggest that serotonergic, dopaminergic, adrenergic, cholinergic, endocannabinoid, opioid, and histaminergic pathways in addition to many neuropeptide pathways (e.g. neuropeptide Y and melanocortins) are implicated in the central control of energy intake and expenditure. Hypothalamic centres are also able to sense peripheral hormones involved in the maintenance of body weight and degree of adiposity, such as insulin and leptin, and fat tissue derived peptides.

Drugs aimed at the pathophysiology associated with insulin dependent Type I diabetes and non-insulin dependent Type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) which is characterized by hypertension and its associated pathologies including atherosclerosis, lipidemia, hyperlipidemia and hypercholesterolemia have been associated with decreased insulin sensitivity which can lead to abnormal blood sugar levels when challenged. Myocardial ischemia and microvascular disease is an established morbidity associated with untreated or poorly controlled metabolic syndrome.

There is a continuing need for novel antiobesity and antidiabetic agents, particularly ones that are well tolerated with few adverse effects.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of obesity and diabetes.

International patent application WO2005/061489 (published after the priority date of the present application) discloses heterocyclic derivatives as GPR119 receptor agonists.

The present invention relates to agonists of GPR119 which are useful for the treatment of obesity e.g. as peripheral regulators of satiety, and for the treatment of diabetes.

### SUMMARY OF THE INVENTION

Compounds of formula (I):

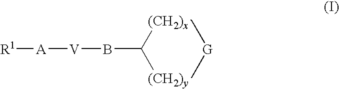

or pharmaceutically acceptable salts thereof, are agonists of GPR119 and are useful as for the prophylactic or therapeutic treatment of obesity and diabetes.

### DETAILED DESCRIPTION OF THE INVENTION

The present invention is directed to a compound of formula (I), or a pharmaceutically acceptable salt thereof:

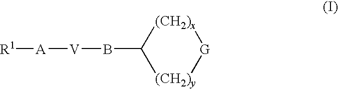

wherein V is a 5-membered heteroaryl ring containing up to four heteroatoms selected from O, N and S, which is optionally substituted by C₁₋₄ alkyl;

A is —CH═CH— or (CH₂)ₙ;

B is —CH═CH— or (CH₂)ₙ, where one of the CH₂ groups may be replaced by O, NR⁵, S(O)ₘ, C(O), C(O)NR⁵, CH(NR⁵R⁵⁵), CH(OH), C(O)O, C(O)S, SC(O) or OC(O);

n is independently 0, 1, 2 or 3;

m is independently 0, 1 or 2;

x is 0, 1, 2 or 3;

y is 1, 2, 3, 4 or 5;

with the proviso that x+y is 2, 3, 4 or 5;

G is CHR¹² or NR²;

R¹ is phenyl or a 5- or 6-membered heteroaryl group containing up to four heteroatoms selected from O, N and S, any of which may be optionally substituted by one or more substituents selected from halo, C₁₋₄ alkyl, C₁₋₄ fluoroalkyl, C₁₋₄ hydroxyalkyl, C₂₋₄ alkenyl, C₂₋₄ alkynyl, C₃₋₇ cycloalkyl, aryl, OR⁶, CN, NO₂, —(CH₂)ⱼ—S(O)ₘR⁶, —(CH₂)ⱼ—C(O)NR⁶R⁶⁶, NR⁶R⁶⁶, NR¹⁰C(O)R⁶, NR¹⁰C(O)NR⁶R⁶⁶, NR¹⁰SO₂R⁶, SO₂NR⁶R⁶⁶, C(O)R¹⁰, C(O)OR¹⁰, —(CH₂)ⱼ-(4- to 7-membered heterocyclyl) or —(CH₂)ⱼ-(5- to 6-membered heteroaryl); provided that R¹ is not optionally substituted 3- or 4-pyridyl, 4- or 5-pyrimidinyl or 2-pyrazinyl;

j is 0, 1 or 2;

R² is C(O)OR³, C(O)NR³R¹³, C₁₋₄alkylene-C(O)OR³, C(O)C(O)OR³, S(O)₂R³, C(O)R³ or P(O)(O-Ph)₂; or heterocyclyl or heteroaryl, either of which may optionally be substituted by one or two groups selected from C₁₋₄alkyl, C₁₋₄alkoxy or halogen;

R³ is C₁₋₈ alkyl, C₂₋₈ alkenyl or C₂₋₈ alkynyl, any of which may be optionally substituted by one or more halo atoms, NR⁴R⁴⁴, OR⁴, C(O)OR⁴, OC(O)R⁴ or cyano, and may contain a CH₂ group that is replaced by O or S; or C₃₋₇ cycloalkyl, aryl, heterocyclyl, heteroaryl, C₁₋₄ alkyleneC₃₋₇ cycloalkyl, C₁₋₄ alkylenearyl, C₁₋₄ alkyleneheterocyclyl or C₁₋₄ alkyleneheteroaryl, any of which may be substituted with one or more substituents selected from halo, C₁₋₄ alkyl, C₁₋₄ fluoroalkyl, OR⁴, CN, NR⁴R⁴⁴, SO₂Me, NO₂ or C(O)OR⁴;

R⁴ and R⁴⁴ are independently hydrogen or C₁₋₄alkyl; or, taken together, R⁴ and R⁴⁴ may form a 5- or 6-membered heterocyclic ring;

R⁵ and R⁵⁵ independently represent hydrogen or C₁₋₄ alkyl;

R⁶ and R⁶⁶ are independently hydrogen or C₁₋₄ alkyl, which may optionally be substituted by halo, hydroxy, C₁₋₄ alkyloxy-, C₁₋₄ alkylthio-, C₃₋₇ heterocyclyl, —C(O)OR¹⁴ or N(R¹⁰)₂; or C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR⁹, CN, SO₂CH₃, N(R¹⁰)₂ and NO₂; or, taken together, R⁶ and R⁶⁶ may form a 4- to 6-membered heterocyclic ring optionally substituted by hydroxy, C₁₋₄ alkyl or C₁₋₄ hydroxyalkyl and optionally containing a further heteroatom selected from O and NR¹⁰, or R⁶⁶ is C₁₋₄ alkyloxy-;

R⁹ is hydrogen, C₁₋₂ alkyl or C₁₋₂ fluoroalkyl;

R¹⁰ are independently hydrogen or C₁₋₄ alkyl; or a group N(R¹⁰)₂ may form a 4- to 7-membered heterocyclic ring optionally containing a further heteroatom selected from O and NR¹⁰;

R¹² is C₃₋₆alkyl; and

R¹³ and R¹⁴ are independently hydrogen or C₁₋₄alkyl.

The molecular weight of the compounds of formula (I) is preferably less than 800, more preferably less than 600, especially less than 500.

In the compounds of formula (I) V is preferably a 5-membered heteroaryl ring containing up to three heteroatoms selected from O, N and S of the formula:

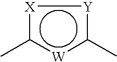

wherein W, X and Y represent the positions of the heteroatom(s) or otherwise represent CH.

Particular heterocyclic rings which V may represent include oxadiazole, oxazole, isoxazole, thiadiazole, thiazole, imidazole and pyrazole. A particular V group is oxadiazole e.g. 1,2,4-oxadiazole.

Suitably at least two of W, X and Y represent N.

Preferably two of W, X and Y are N, and the other is O.

W is preferably N.

Preferably the groups A and B do not both represent a bond, i.e. n is not 0 in each case.

A is preferably (CH₂)ₙ wherein n is preferably 0, 1 or 2, more preferably 0.

Suitably B is —CH═CH— or (CH₂)ₙ, where one of the CH₂ groups may be replaced by O, NR⁵, S(O)ₘ, C(O), C(O)NR⁵, CH(NR⁵R⁵⁵), C(O)O, C(O)S, SC(O) or OC(O).

B is preferably (CH₂)ₙ wherein n is preferably 1, 2 or 3, more preferably 2 or 3, especially 2.

When one of the CH₂ groups in B is replaced, it is preferably replaced by O or NR⁵, most preferably by O. In one embodiment of the invention a CH₂ group in B is replaced. In a second embodiment of the invention a CH₂ group in B is not replaced

R¹ is suitably phenyl or a 5- or 6-membered heteroaryl group containing up to four heteroatoms selected from O, N and S, any of which may be optionally substituted by one or more substituents selected from halo, C₁₋₄ alkyl, C₁₋₄ fluoroalkyl, C₁₋₄ hydroxyalkyl, C₂₋₄ alkenyl, C₂₋₄ alkynyl, C₃₋₇ cycloalkyl, aryl, OR⁶, CN, NO₂, S(O)ₘR⁶, C(O)NR⁶R⁶⁶, NR⁶R⁶⁶, NR¹⁰C(O)NR⁶R⁶⁶, NR¹⁰C(O)R⁶, NR¹⁰SO₂R⁶, SO₂NR⁶R⁶⁶, C(O)R¹⁰, C(O)OR¹⁰, a 4- to 7-membered heterocyclyl group or a 5- or 6-membered heteroaryl group; provided that R¹ is not optionally substituted 3- or 4-pyridyl, 4- or 5-pyrimidinyl or 2-pyrazinyl.

In one embodiment of the invention R¹ is phenyl or a 5- or 6-membered heteroaryl group containing up to four heteroatoms selected from O, N and S, any of which may be optionally substituted by one or more substituents selected from halo, C₁₋₄ alkyl, C₁₋₄ fluoroalkyl, C₁₋₄ hydroxyalkyl, C₂₋₄ alkenyl, C₂₋₄ alkynyl, C₃₋₇ cycloalkyl, aryl, OR⁶, CN, NO₂, S(O)ₘR⁶, C(O)NR⁶R⁶⁶, NR⁶R⁶⁶, NR¹⁰C(O)R⁶, NR¹⁰SO₂R⁶, SO₂NR⁶R⁶⁶, C(O)R¹⁰, C(O)OR¹⁰, 4- to 7-membered heterocyclyl or 5- to 6-membered heteroaryl; provided that R¹ is not optionally substituted 3- or 4-pyridyl, 4- or 5-pyrimidinyl or 2-pyrazinyl.

R¹ is preferably phenyl or a 6-membered heteroaryl group containing up to two N heteroatoms either of which rings may optionally be substituted, especially optionally substituted phenyl. When R¹ is phenyl or a 6-membered heteroaryl group it is preferably substituted in the meta and/or para positions.

Examples of R¹ heteroaryl groups include oxazolyl, isoxazolyl, thienyl, pyrazolyl, imidazolyl, furanyl, pyridazinyl or 2-pyridyl.

Preferred substituent groups for R¹ are halo, C₁₋₄ alkyl, C₁₋₄ fluoroalkyl, C₂₋₄ alkenyl, C₂₋₄ alkynyl, CN, S(O)R⁶, NR¹⁰C(O)NR⁶R⁶⁶, C(O)NR⁶R⁶⁶, SO₂NR⁶R⁶⁶, NR¹⁰SO₂R⁶, COR¹⁰, C(O)OR¹⁰ or a 5- or 6-membered heteroaryl group; especially halo e.g. fluoro or chloro, C₁₋₄ alkyl, C₁₋₄ fluoroalkyl, C₂₋₄alkenyl, C₂₋₄alkynyl, CN, S(O)ₘR⁶, NR¹⁰C(O)NR⁶R⁶⁶, C(O)NR⁶R⁶⁶ or SO₂NR⁶R⁶⁶ or a 5-membered heteroaryl group; in particular fluoro, chloro, methyl, S(O)ₘR⁶ e.g. where m is 1 or 2, NR¹⁰C(O)NR⁶⁶, C(O)NR⁶⁶ or SO₂NR⁶⁶ or a 5-membered heteroaryl group.

A further group of substituents for R¹ are halo, C₁₋₄ alkyl, C₁₋₄ fluoroalkyl, C₂₋₄ alkenyl, C₂₋₄ alkynyl, CN, S(O)ₘR⁶, C(O)NR⁶⁶, SO₂NR⁶R⁶⁶, NR¹⁰SO₂R⁶, COR¹⁰, C(O)OR¹⁰ or a 5- or 6-membered heteroaryl group; especially halo e.g. fluoro or chloro, C₁₋₄ alkyl, C₁₋₄ fluoroalkyl, C₂₋₄ alkenyl, C₂₋₄ alkynyl, CN, S(O)ₘR⁶, C(O)NR⁶R⁶⁶ or SO₂NR⁶R⁶⁶ or a 5-membered heteroaryl group; in particular fluoro, chloro, methyl, S(O)ₘR⁶ e.g. where m is 1 or 2, C(O)NR⁶⁶ or SO₂NR⁶R⁶⁶.

Suitably j is 0 or 1. In one embodiment of the invention j represents 0. In a second embodiment of the invention j represents 1.

G is preferably NR².

In one embodiment of the invention x+y is 2, 3, or 4. Suitably, x is 1 or 2 and y is 1 or 2. In a preferred embodiment of the invention x and y each represent 1. In a more preferred embodiment of the invention x and y each represent 2.

R² is preferably C(O)OR³, C(O)NR³R¹³, C₁alkylene-C(O)OR³, C(O)C(O)OR³, heterocyclyl, heteroaryl, S(O)₂R³, C(O)R³ or P(O)(O-Ph)₂; especially C(O)OR³, C(O)NR³R¹³, C₁₋₄alkylene-C(O)OR³, heteroaryl, S(O)₂R³ or C(O)R³; in particular C(O)OR³, C(O)NR³R¹³, heteroaryl, S(OR³ or C(O)R³. More preferably, R² is C(O)OR³, C(O)NR³R¹³ or heteroaryl. R² is most preferably C(O)OR³. When R² is heteroaryl the heteroaryl ring it is preferably a 5- or 6-membered heteroaryl ring containing up to 4 heteroatoms selected from N, O and S, suitably pyridinyl e.g. 2-pyridinyl, oxadiazolyl or pyrimidinyl, preferably pyrimidinyl, especially pyrimidin-2-yl. Heterocyclic rings that R² may represent included 4- to 7-membered rings containing 1 or 2 N or O atoms, examples of heterocyclic rings that R² may represent include azetidine, pyrrolidine, piperidine and piperazine. R² heterocyclyl groups may also contain additional heteroatoms, e.g. morpholine.

Preferably R³ represents C₁₋₈ alkyl, C₂₋₈ alkenyl or C₂₋₈ alkynyl, optionally substituted by one or more halo atoms, NR⁴R⁴⁴, OR⁴, C(O)OR⁴, OC(O)R⁴ or cyano, and which may contain a CH₂ group that is replaced by O or S; or a C₃₋₇ cycloalkyl, aryl or C₁₋₄ alkylC₃₋₇ cycloalkyl, any of which may be substituted with one or more substituents selected from halo, C₁₋₄ alkyl, C₁₋₄ fluoroalkyl, OR⁴, CN, NR⁴R⁴⁴, NO₂ or C(O)OC₁₋₄alkyl. More preferably R³ represents C₁₋₈ alkyl, C₂₋₈ alkenyl or C₂₋₈ alkynyl optionally substituted by one or more halo atoms or CN, and which may contain a CH₂ group that may be replaced by O or S; or C₃₋₇ cycloalkyl or aryl, either of which may be substituted with one or more substituents selected from halo, C₁₋₄ alkyl, C₁₋₄ fluoroalkyl, OR⁴, CN, NR⁴R⁴⁴, NO₂ or C(O)OC₁₋₄ alkyl. Most preferred R³ groups are C₂₋₅ alkyl e.g. C₂₋₅ alkyl, optionally substituted by one or more halo atoms or CN, and may contain a CH₂ group that is replaced by O or S; or C₃₋₅cycloalkyl optionally substituted by C₁₋₄ alkyl. In one embodiment of the invention the group represented by R³ is unsubstituted. Exemplary R³ groups include ethyl, n-propyl, isopropyl, 1-methyl-cycloprop-1-yl, cyclopropylmethyl-, 1-methyl-cycloprop-1-ylmethyl-, tert-butyl-, cyclobutyl and 1-methyl-cyclobut-1-yl.

Suitably R⁴ and R⁴⁴ are independently hydrogen or methyl, especially methyl.

Suitably R⁵ represents hydrogen or methyl, especially methyl.

Suitably R⁶ and R⁶⁶ are independently hydrogen or C₁₋₄ alkyl, which may optionally be substituted by halo e.g. fluoro, hydroxy, C₁₋₄ alkyloxy-, C₁₋₄ alkylthio-, C₃₋₇ heterocyclyl or N(R¹⁰)₂; or C₃₋₇ cycloalkyl, aryl, heterocyclyl or heteroaryl, wherein the cyclic groups may be substituted with one or more substituents selected from halo, C₁₋₄alkyl, C₁₋₄ fluoroalkyl, OR⁹, CN, SO₂CH₃, NR¹⁰)₂ and NO₂; or, taken together, R⁶ and R⁶⁶ may form a 5- or 6-membered heterocyclic ring optionally substituted by hydroxy, C₁₋₄ alkyl or C₁₋₄ hydroxyalkyl; or R⁶⁶ is C₁₋₄ alkyloxy-.

In one embodiment of the invention R⁶ and R⁶⁶ are independently hydrogen or C₁₋₄ alkyl, which may optionally be substituted by halo e.g. fluoro, hydroxy, C₁₋₄ alkyloxy-, C₁₋₄ alkylthio-, C₃₋₇ heterocyclyl, —(O)OR¹⁴ or N(R¹⁰); or C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR⁹, CN, SO₂CH₃, N(R¹⁰) and NO₂.

When the group R⁶ is attached to a sulfoxide or sulfone, R⁶ is preferably optionally substituted C₁₋₄ alkyl or optionally substituted C₃₋₇ cycloalkyl, more preferably optionally substituted C₁₋₄ alkyl. When the group R⁶ is attached to C(O)N, R⁶ is preferably hydrogen, optionally substituted C₁₋₄ alkyl or optionally substituted C₃₋₇ cycloalkyl, more preferably optionally substituted C₁₋₄alkyl. Exemplary R⁶ groups include methyl, ethyl, propyl, butyl, hydrogen, cyclopropyl, methyoxymethyl, methoxyethyl, methoxypropyl, hydroxyethyl, hydroxypropyl, tetrahydropyran and piperidine. Exemplary R⁵⁶ groups include hydrogen, methyl and ethyl. Exemplary rings formed by R⁶ and R⁶⁶ include morpholine, pyrrolidine, azetidine, piperazine and piperidine.

R⁹ is preferably C₁₋₂ alkyl or C₁₋₂ fluoroalkyl.

Suitably R¹⁰ is hydrogen, methyl or tert-butyl.

Suitably R¹² is pentyl.

Suitably R¹⁴ is hydrogen or methyl.

R¹³ and R¹⁵ are preferably independently hydrogen or methyl.

m is preferably 1 or 2.

One group of compounds are those of formula (Ia):

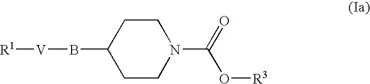

wherein:

B represents (CH₂), where n is 2 or 3 and one of the CH₂ groups may be replaced by O or NR⁵;

R¹, V, R³ and R⁵ are as described previously for compounds of formula (I).

For compounds of formula (Ia), suitably V represents oxadiazole, e.g. 1,2,4-oxadiazole.

A group of compounds of formula (Ia) are those of formula (Ib):

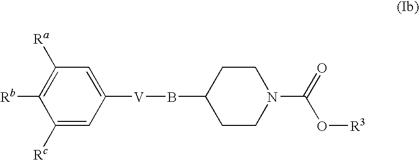

wherein:

R^{a} and R^{c} independently represent hydrogen, fluorine, chlorine, methyl or CN;

R^{b} represents S(O)ₘR⁶, C(O)NR⁶R⁶⁶, SO₂NR⁶R⁶⁶, NR¹⁰C(O)R⁶, NR¹⁰SO₂R⁶, NR¹⁰C(O)NR⁶R⁶⁶ or 5-membered heteroaryl;

R³ represents C₂₋₅ alkyl or C₃₋₅ cycloalkyl which may optionally be substituted by methyl;

m represents 1 or 2;

R⁶ and R⁶⁶ independently represent hydrogen or C₁₋₄ alkyl which may optionally be substituted by hydroxyl or NH₂, alternatively R⁶ and R⁶⁶ taken together may form a 5- or 6 membered heterocyclic ring optionally substituted with OH or CH₂OH; and

R¹⁰ are independently hydrogen or C₁₋₄ alkyl; or a group N(R¹⁰)₂ may form a 4- to 7-membered heterocyclic ring optionally containing a further heteroatom selected from O and NR¹⁰.

For compounds of formula (Ib), suitably B represents —CH₂—O—.

For compounds of formula (Ib), suitably R^{b} represents S(O)ₘR⁶, C(O)NR⁶R⁶⁶, NR¹⁰C(O)NR⁶R⁶⁶, 5-membered heteroaryl or SO₂NR⁶R⁶⁶. Alternatively for compounds of formula (Ib) R^{b} represents NR¹⁰C(O)R⁶ or NR¹⁰SO₂R⁶.

While the preferred groups for each variable have generally been listed above separately for each variable, preferred compounds of this invention include those in which several or each variable in formulae (I) to (Ib) is selected from the preferred, more preferred or particularly listed groups for each variable. Therefore, this invention is intended to include all combinations of preferred, more preferred and particularly listed groups.

Specific compounds of the invention which may be mentioned are those included in the Examples and pharmaceutically acceptable salts thereof.

The following provisos may optionally be used (individually or in any combination) to exclude certain compounds from the scope of the invention:

i) when R¹ represents fluorophenyl or difluorophenyl, A and B represent a bond, x represents 1, y represents 3, suitably G does not represent NC(O)O-fluorophenyl.

ii) when G represents CHR¹² and R¹² is pentyl, x represents 2, y represents 2, A represents a bond and V represents 1,3,4-oxadiazole, suitably R¹ does not represent phenyl substituted by 3-dimethylamino-pyrrolidin-1-yl.

iii) when R¹ represents phenyl, A represents —CH₂—, B represents a bond, x represents 0, y represents 4 and G represents NR² suitably R² does not represent S(O)₂R³.

iv) when R¹ represents substituted furan, A and B represent a bond, x represents 0, y represents 4 and G represents NR² suitably R² does not represent S(O)₂—CH₂-cyclohexyl.

v) when R¹ represents 4-methanesulphonylphenyl, A represents a bond, B represents —CH₂CH₂—, x represents 2 and y represents 2, suitably G does not represent N-cyclopropyl.

vi) when x represents 0, y represents 3 and B represents a bond, suitably G does not represent NC(O)R³.

As used herein, unless stated otherwise, “alkyl” as well as other groups having the prefix “alk” such as, for example, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

The term “fluoroalkyl” includes alkyl groups substituted by one or more fluorine atoms, e.g. CH₂F, CHF₂ and CF₃.

The term “cycloalkyl” means carbocycles containing no heteroatoms, and includes monocyclic and bicyclic saturated and partially saturated carbocycles. Examples of cycloalkyl include cyclopropyl, cyclobutyl, cyclopentyl, cyclohexyl and cycloheptyl. Examples of partially saturated cycloalkyl groups include cyclohexene and indane. Cycloalkyl groups will typically contain 3 to 10 ring carbon atoms in total, e.g. 3 to 6, or 8 to 10.

The term “halo” includes fluorine, chlorine, bromine, and iodine atoms (in particular fluorine or chlorine).

The term “aryl” includes phenyl and naphthyl, in particular phenyl.

Unless otherwise indicated the term “heterocyclyl” and “heterocyclic ring” includes 4- to 10-membered monocyclic and bicyclic saturated rings, e.g. 4- to 7-membered monocyclic saturated rings, containing up to three heteroatoms selected from N, O and S. Examples of heterocyclic rings include oxetane, tetrahydrofuran, tetrahydropyran, oxepane, oxocane, thietane, tetrahydrothiophene, tetrahydrothiopyran, thiepane, thiocane, azetidine, pyrrolidine, piperidine, azepane, azocane, [1,3]dioxane, oxazolidine, piperazine, and the like. Other examples of heterocyclic rings include the oxidised forms of the sulfur-containing rings. Thus, tetrahydrothiophene 1-oxide, tetrahydrothiophene 1,1-dioxide, tetrahydrothiopyran 1-oxide, and tetrahydrothiopyran 1,1-dioxide are also considered to be heterocyclic rings.

Unless otherwise stated, the term “heteroaryl” includes mono- and bicyclic 5- to 10-membered, e.g. monocyclic 5- or 6-membered, heteroaryl rings containing up to 4 heteroatoms selected from N, O and S. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl and triazinyl. Bicyclic heteroaryl groups include bicyclic heteroaromatic groups where a 5- or 6-membered heteroaryl ring is fused to a phenyl or another heteroaromatic group. Examples of such bicyclic heteroaromatic rings are benzofuran, benzothiophene, indole, benzoxazole, benzothiazole, indazole, benzimidazole, benzotriazole, quinoline, isoquinoline, quinazoline, quinoxaline and purine. Preferred heteroaryl groups are monocyclic 5- or 6-membered, heteroaryl rings containing up to 4 heteroatoms selected from N, O and S.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above formula (I) is shown without a definitive stereochemistry at certain positions. The present invention includes all stereoisomers of formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of formula (I) exists, the present invention includes any possible tautomers and pharmaceutically acceptable salts thereof, and mixtures thereof, except where specifically drawn or stated otherwise.

When the compound of formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylainine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like

Since the compounds of formula (I) are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below, in which, for illustrative purposes, —V— is shown as a group of the formula:

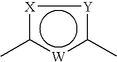

and R¹, A, B, x, y, G, W, X and Y are as defined above.

The compounds of formula (I), in which X=N, Y=O and W=N, may be prepared according to the method illustrated in Scheme 1. The nitriles of formula 2 are either commercially available or can be synthesised using known techniques. Compounds of formula 2 are treated with hydroxylamine in a suitable solvent, such as ethanol-water, at elevated temperature, to afford amidoximes of formula 3 (synthesis of amidoximes is further described by A. R. Martin et al, J. Med. Chem., 2001, 44, 1560). Compounds of formula 3 are subsequently condensed with acids of formula 4, which are themselves either commercially available or can be readily synthesised using known techniques. The condensation firstly entails activation of compounds of formula 4 by, for example, formation of the mixed anhydride, in which the acid is treated with a chloroformate, such as isobutylchloroformate, in the presence of a suitable base, such as triethylamine, in a suitable solvent, such as THF or toluene, followed by addition of compounds of formula 3. Alternatively, compounds of formula 4 may be activated by conversion to the acid halide, generated by treatment of the acid with, for example, oxalyl chloride in a suitable solvent, such as CH₂Cl₂-DMF. The intermediates arising from the condensation of amidoximes of formula 3 and acids of formula 4 are dissolved in an appropriate solvent, such as toluene or xylene, and heated under reflux, with concomitant removal of water by Dean-Stark apparatus or by molecular sieves, to form oxadiazoles of formula (I). Alternatively, amidoximes of formula 3 can firstly be treated with a suitable base, for example sodium hydride, in an appropriate solvent, such as THF, and subsequently esters of formula 5. Heating of this mixture also generates oxadiazoles of formula (I) (this process is further illustrated by R. H. Mach et al, Bioorg. Med. Chem., 2001, 9, 3113).

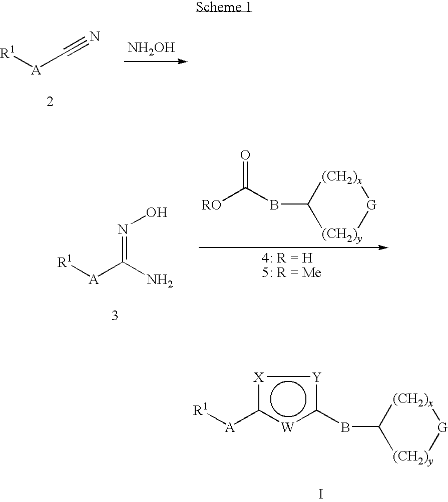

Compounds of formula (I) in which X=O, Y=N and W=N may be prepared according to the method outlined in Scheme 2. The nitrites of formula 6 are either commercially available or can be synthesised using known techniques. These are converted to the corresponding amidoximes of formula 7, as described above, and subsequently condensed with acids of formula 8, which are commercially available or can readily be synthesised by those skilled in the art. This condensation is performed in a fashion analogous to that described in Scheme 1, to afford the corresponding oxadiazoles of formula (I).

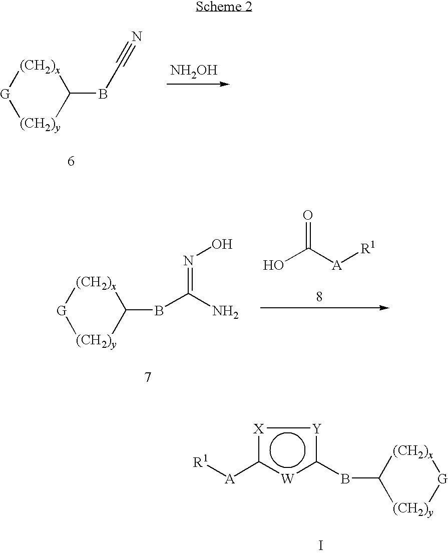

Compounds of formula (I) in which X=N, Y=N and W=O can be synthesised as outlined in Scheme 3. The acyl chlorides of formula 9 are either commercially available or may be synthesised using known methods. The acid hydrazides of formula 10 can be readily obtained by, for example, treating an ethanolic solution of the corresponding ester with hydrazine (for further details see K M. Kahn et al, Bioorg. Med. Chem., 2003, 11, 1381). Treating the acyl chlorides of formula 9 with the acid hydrazides of formula 10 in a suitable solvent, such as pyridine, affords compounds of formula 11 (further illustrated by V. N. Kerr et al, J. Am. Chem. Soc., 1960, 82, 186), which are then converted by POCl₃ at elevated temperature to compounds of formula (I) (this process is further described by S-A. Chen et al, J. Am. Chem. Soc., 2001, 123, 2296). Similarly, compounds of formula (I) where X=Y=W=N can be prepared via the condensation of the amidrazone analogue of 10 with the appropriate activated carboxylic acid derivative, such as 9. The reactive groups in this reaction may be exchanged, i.e. an amidrazone of formula R¹-A-C(═NH)NHNH₂ can form a compound of formula (I) by condensation with an activated carboxylic acid derivative LG-C(═O-—-B-cycle where LG is halogen or oxycarbonyl (P. H. Olesen et al., J. Med. Chem., 2003, 46, 3333-3341).

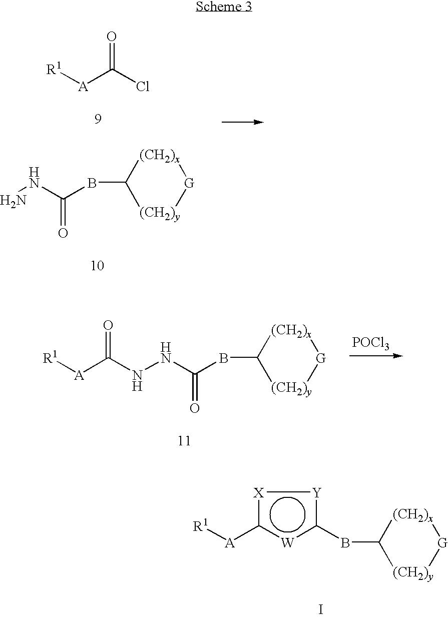

Compounds of formula (I) where X=N, Y=N, and W=S can also be prepared from compounds of formula 11 by heating with Lawesson's reagent in a suitable solvent, such as toluene or acetonitrile (D. Alker et al., J. Med. Chem., 1989, 32, 2381-2388). Compounds of formula (I) where X=S, Y=N and W=N can be formed from compounds of formula 12 (Scheme 4) which are commercially available, or can be readily synthesised from the corresponding carbonyl compound and Lawesson's reagent under standard conditions. Treating a compound of formula 12 with a compound of formula 13 in a suitable solvent such as dichloromethane at about 20° C. gives compounds of formula 14. Compounds of formula 13 can be obtained by treating the corresponding dimethylamide with Meerwein's reagent (for details see M. Brown U.S. Pat. No. 3,092,637). Compounds of formula 14 are then cyclised using hydroxylamine-O-sulfonic acid in the presence of a base, such as pyridine, in a suitable solvent such as methanol (for further details, see A. MacLeod et al, J. Med. Chem., 1990, 33, 2052).

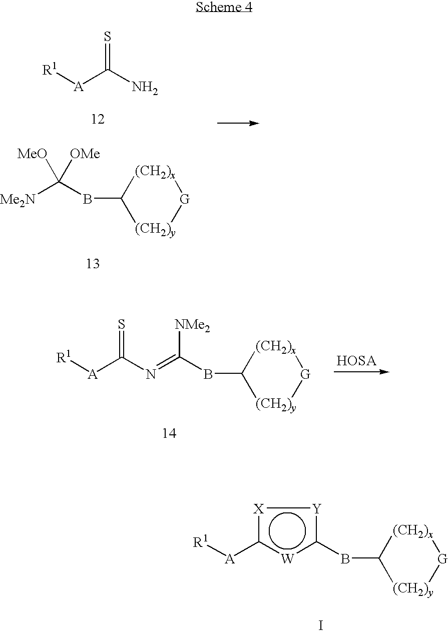

The regioisomeric derivatives of formula (I), where X=N, Y=S and W=N, can be formed in a similar manner by reversing the functionality of the reactants so the R¹ fragment contains the acetal moiety and the G containing cycle fragment contains the thiocarbonyl.

Compounds of formula (I) where W=O, X=N and Y=CH can be formed from compounds of formula 15 (Scheme 5). Compounds of formula 15 are commercially available or synthesised using known techniques. Chlorides of formula 16 are commercially available, or can readily be formed by chlorinating the corresponding ketone using standard conditions, for example, bubbling chlorine gas through a methanol solution of the ketone (for further details see R. Gallucci & R. Going, J. Org. Chem., 1981, 46, 2532). Mixing a compound of formula 15 with a chloride of formula 16 in a suitable solvent, such as toluene, with heating, for instance at about 100° C. gives compounds of formula (I) (for further information, see A. Hassner et al, Tetrahedron, 1989, 45, 6249). Compounds of formula (I) where W=O, X=CH and Y=N can be formed is a similar fashion by reversing the functionality of the reactants so the R¹ fragment contains the haloketone moiety and the G containing cycle fragment contains the C(O)NH₂.

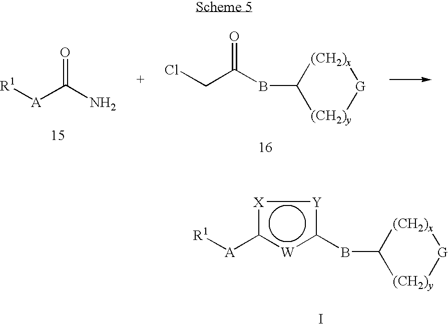

Alternatively, compounds of formula (I) where X=S, W=N and Y=CH can also be formed from compounds of formula 16. Heating an compound of formula 15 with phosphorus pentasulfide, followed by the addition of a compound of formula 16 followed by further heating gives compounds of formula (I) (for further details, see R. Kurkjy & E. Brown, J. Am. Chem. Soc., 1952, 74, 5778). The regioisomeric compounds where X=CH, W=N and Y=S can be formed is a similar fashion by reversing the functionality of the reactants, so the R¹ fragment contains the haloketone moiety and the G containing cycle fragment contains the C(O)NH₂.

Compounds of formula I where W=N, X=O and Y=CH can be formed from compounds of formula 15 and formula 17 (Scheme 6) under similar conditions to those outlined for Scheme 5. Compounds of formula I where W=S, X=N and Y=CH can also be formed from compounds of formula 15 and formula 17 using the conditions involving phosphorus pentasulfide described above.

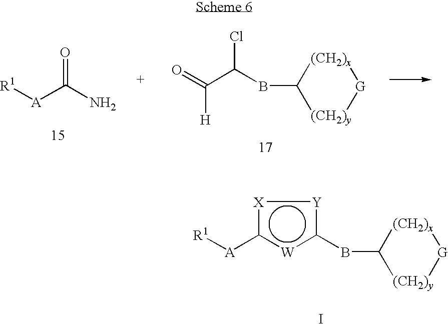

Compounds of formula (I) where X=O, Y=N and W=CH, and where X=N, Y═O and W=CH and can be formed from compounds of formula 20 (Scheme 7). Acylation of compounds of formula 18 with a compound of formula 19, where Q is alkoxide or chloride, can occur under standard conditions, for example, deprotonation of ketone 18 with a suitable base, such as lithium diisopropylamide or potassium ethoxide, in a suitable solvent, such as tetrahydrofuran, generally at low temperature. Treatment of compounds of formula 20 with hydroxylamine, in a suitable solvent, such as ethanol, at elevated temperature, for example 75° C., yields compounds of formula (I) as a mixture of both regioisomers of the isoxazole. Using standard separation techniques, such as chromatography on silica gel, the individual isomers can be isolated (for further details, see M. Rowley et al, J. Med. Chem., 1997, 40, 2374).

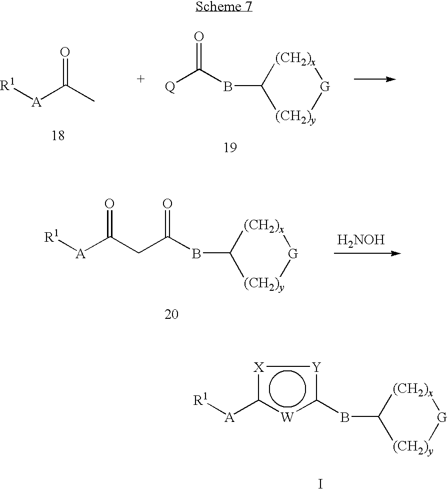

Compounds of formula (I) where X=S, Y=N and W=CH can be formed by hydrogenation of a compound of formula (I) where X=O, Y=N and W=CH, with platinum oxide in a suitable solvent such as ethanol, followed by heating with phosphorus pentasulfide to give compounds of formula (I) where X=S, Y=N and W=CH (for further details, see G. Wiegand et al, J. Med. Chem., 1971, 14, 1015). For details of the synthesis of the regioisomer where X=N, Y=S and W=CH also see G. Wiegand ibid.

Compounds of formula (I) where X=N, Y=N and W=CH can be formed from compounds of formula 20. Treatment of compounds of formula 20 with hydrazine in a suitable solvent, such as methanol, would give rise to compounds of formula (I) where X=N, Y=N and W=CH (this process is further illustrated by R. Baker et al, J. Med. Chem., 1997, 40, 2374).

Compounds of formula (I) in which X=CH, Y=N and W=N can be synthesised as described in Scheme 8. Bromides of formula 23 are either commercially available or may be synthesised from the corresponding ketone by, for example, treating an aqueous solution of the ketone with Br₂ and HBr (as described by J. Y. Becker et al, Tetrahedron Lett., 2001, 42, 1571). The amidines of formula 22 may be synthesised by known methods, for example by treatment of the corresponding alkyl imidates of formula 21 with ammonia in a suitable solvent, such as ethanol (as detailed by D. A. Pearson et al, J. Med. Chem., 1996, 39, 1372). The imidates of formula 21 may in turn be generated by, for example, treatment of the corresponding nitrile with HCl in a suitable solvent, such as methanol (for further details see J. P. Lokensgard et al, J. Org. Chem., 1985, 50, 5609). Reaction of amidines of formula 22 with bromides of formula 23 in a suitable solvent, such as DMF, affords compounds of formula (I) (illustrated by N. J. Liverton et al, J. Med. Chem., 1999, 42, 2180).

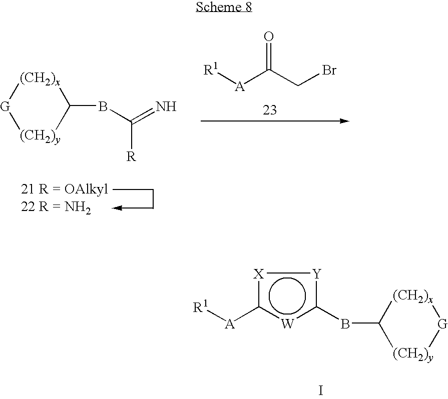

The regioisomeric compounds where X=N, Y=CH and W=N can be formed in a similar fashion by reversing the functionality of the reactants, so the R¹ fragment contains the amidine moiety and the R² fragment contains the bromide.

Compounds of formula (I) in which X=CH, Y=CH and W=N can be synthesised as illustrated in Scheme 9. Diketones of formula 25 are readily accessible by, for example, the condensation of ketones of formula 24, which are commercially available or are readily synthesised using known techniques, with bromides of formula 23 in a suitable solvent, such as benzene using an appropriate catalyst. Illustrative examples are described by O. G. Kulinkovich et al, Synthesis, 2000, 9, 1259. Using a Paal-Knorr reaction, diketones of formula 25 may be treated with, for example, ammonium carbonate in a suitable solvent, such as ethanol at elevated temperature (for further details see R. A. Jones et al, Tetrahedron, 1996, 52, 8707) to afford compounds of formula (I).

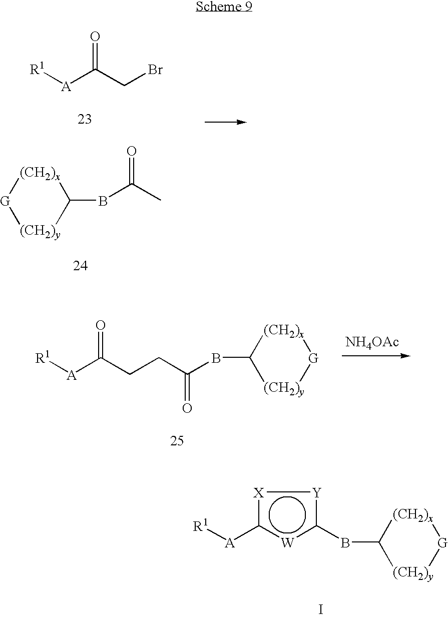

Compounds of formula (I) in which R² contains either a carbamate or a sulfonamide group may be synthesised as described in Scheme 10. Compounds of formula 26, in which P represents a suitable protecting group, for example tert-butoxycarbonyl (Boc), may be synthesised as outlined in Schemes 1-9 above. The protecting group is firstly removed under suitable conditions to afford compounds of formula 27. In the case of the Boc group this can be achieved by treatment of compounds of formula 26 with a suitable acid, such as trifluoroacetic acid, in an appropriate solvent, such as CH₂Cl₂. Treatment of compounds of formula 27 with chloroformates of formula 28, which are generally commercially available or can be readily synthesised, in a suitable solvent, such as CH₂Cl₂, in the presence of a suitable base, such as triethylamine, affords compounds of formula (I). Similarly, compounds of formula 27 may be reacted with sulfonyl chlorides of formula 29, which are generally commercially available or can readily be synthesised, in a suitable solvent, such as CH₂Cl₂, in the presence of a suitable base, such as triethylamine, to afford compounds of formula (I). Compounds of formula (I) in which R² contains a urea moiety may be prepared by reacting a compound of formula 27 with an isocyanate of formula —N—R³. Furthermore, compounds of formula (I) in which R² a heteroaryl group may be prepared by reacting the amine 27 with the appropriate heteroaryl chloride or bromide under Pd(0) catalysis in the presence of a suitable ligand and base (Urgaonkar, S.; Hu, J.-H.; Verkade, J. G. J. Org. Chem. 2003, 68, 8416-8423).

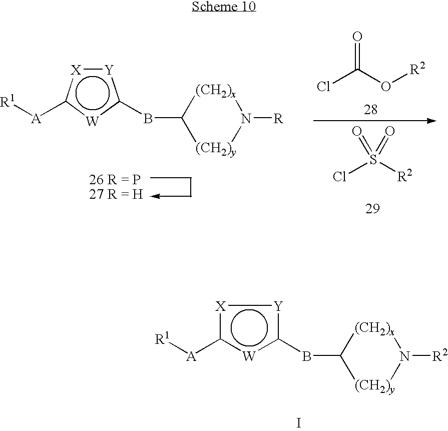

Compounds of formula (I) in which R² contains an amide group may be synthesised from compounds of formula 27 and a suitable acid (R³COOH), or activated derivative thereof, in an amide bond forming reaction.

Compounds of formula (I) where B contains a NR⁵ group where R⁵ is hydrogen can be further transformed into compounds of formula (I) where R⁵ is C₁₋₄ alkyl group using standard techniques known to those with skill in the art.

Compounds of the formula (I) where R¹ is pyridyl optionally substituted with CN can be prepared from the corresponding unsubstituted pyridine by the Reissert reaction (Fife, W. K. J. Org. Chem. 1983, 48, 1375-1377). Similar reactions can be used to prepare the compounds where R¹ is pyridyl optionally substituted with halogen (Walters, M. A.; Shay, J. J. Tetrahedron Lett. 1995, 36, 7575-7578). The compounds where R¹ is pyridyl optionally substituted with halogen can be transformed into the corresponding compounds where R¹ is pyridyl optionally substituted with C₁₋₄ alkyl by transition metal-catalysed cross-coupling reactions (Fürstner, A., et al. J. Am. Chem. Soc. 2002, 124, 13856-13863).

Compounds of formula (I) and where X=N, Y=N, U=N and W=CH can be synthesised as shown in Scheme 11 below. Illustrative examples are described by M. Meldal et al Journal of Organic Chemistry (2002), 67(9), 3057-3064. Azides of formula 30 are either commercially available or may be synthesised, for example, from the displacement of the corresponding halides with azide ion using known techniques; or synthesised from the corresponding amine derivative via reaction with sodium nitrite in acidic media. The alkynes of formula 31 may be commercial or synthesised by known methods, for example by reaction of acetylide ions with boranes (see Journal of Organic Chemistry (1981), 46(11) 2311-2314) or aldehydes or ketones.

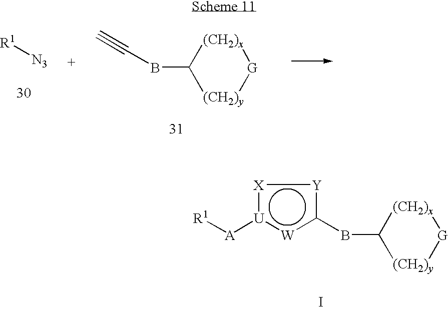

Compounds of formula (I) and where X=N, Y=CH, U=N and W=CH can be synthesised as shown in Scheme 12 by reaction of 1,3-dicarbonyl compounds of formula 33 (or their equivalents, such as enol ethers) with hydrazines of formula 32. The hydrazines of formula 32 may be commercial or synthesised by known methods, for example by reaction of the corresponding amine with sodium nitrite and reacting the resulting diazonium salt with a reducing agent such as sodium sulfite.

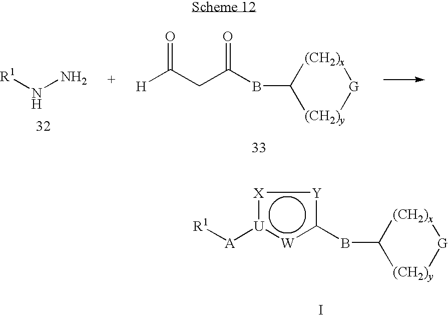

Other compounds of formula (I) may be prepared by methods analogous to those described above or by methods known per se.

Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

Any novel intermediates as defined above are of use in the synthesis of compounds of formula (I) and are therefore also included within the scope of the invention. For example, compounds of formula (XXVII):

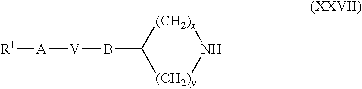

or a salt or protected derivative thereof, wherein the groups R¹, A, V, B, x and y are as defined above for compounds of formula (I). In the compounds of formula (XXVII) when R¹ represents biphenyl, A and B represent a bond, x represents 2, suitably y does not represent 2.

An example compound falling within the scope of formula (XXVII) is 4-[5-(4-methanesulfonylphenyl)-[1,2,4]oxadizol-3-ylmethoxy]piperidine.

As indicated above the compounds of formula (I) are useful as GPR119 agonists, e.g. for the treatment and/or prophylaxis of obesity and diabetes. For such use the compounds of formula (I) will generally be administered in the form of a pharmaceutical composition.

The invention also provides a compound of formula (I), or a pharmaceutically acceptable salt thereof, for use as a pharmaceutical.

The invention also provides a pharmaceutical composition comprising a compound of formula (I), in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, the invention also provides a pharmaceutical composition for the treatment of disease by modulating GPR119, resulting in the prophylactic or therapeutic treatment of obesity, e.g. by regulating satiety, or for the treatment of diabetes, comprising a pharmaceutically acceptable carrier and a non-toxic therapeutically effective amount of compound of formula (I), or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 30 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of formula (I) may be used in the treatment of diseases or conditions in which GPR119 plays a role.

Thus the invention also provides a method for the treatment of a disease or condition in which GPR119 plays a role comprising a step of administering to a subject in need thereof an effective amount of a compound of formula (I), or a pharmaceutically acceptable salt thereof. Diseases or conditions in which GPR119 plays a role include obesity and diabetes. In the context of the present application the treatment of obesity is intended to encompass the treatment of diseases or conditions such as obesity and other eating disorders associated with excessive food intake e.g. by reduction of appetite and body weight, maintenance of weight reduction and prevention of rebound and diabetes (including Type 1 and Type 2 diabetes, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The compounds of the invention may offer advantages over compounds acting via different mechanisms for the treatment of the above mentioned disorders in that they may offer beta-cell protection, increased cAMP and insulin secretion and also slow gastric emptying.

The invention also provides a method for the regulation of satiety comprising a step of administering to a subject in need thereof an effective amount of a compound of formula (I), or a pharmaceutically acceptable salt thereof.

The invention also provides a method for the treatment of obesity comprising a step of administering to a subject in need thereof an effective amount of a compound of formula (I), or a pharmaceutically acceptable salt thereof.

The invention also provides a method for the treatment of diabetes, including Type 1 and Type 2 diabetes, particularly type 2 diabetes, comprising a step of administering to a patient in need thereof an effective amount of a compound of formula (I), or a pharmaceutically acceptable salt thereof.

The invention also provides a method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of formula (I), or a pharmaceutically acceptable salt thereof.

The invention also provides a compound of formula (I), or a pharmaceutically acceptable salt thereof for use in the treatment of a condition as defined above.

The invention also provides the use of a compound of formula (I), or a pharmaceutically acceptable salt thereof, in the manufacture of a medicament for the treatment of a condition as defined above.

In the methods of the invention the term “treatment” includes both therapeutic and prophylactic treatment.

The compounds of formula (I), or pharmaceutically acceptable salts thereof, may be administered alone or in combination with one or more other therapeutically active compounds. The other therapeutically active compounds may be for the treatment of the same disease or condition as the compounds of formula (I) or a different disease or condition. The therapeutically active compounds may be administered simultaneously, sequentially or separately.

The compounds of formula (I) may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, mixed PPAR-α/γ agonists, RXR agonists, fatty acid oxidation inhibitors, α-glucosidase inhibitors, dipeptidyl peptidase IV inhibitors, GLP-1 agonists e.g. GLP-1 analogues and mimetics, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of formula (I), or a pharmaceutically acceptable salt thereof, and at least one other antiobesity agent represents a further aspect of the invention.

The present invention also provides a method for the treatment of obesity in a mammal, such as a human, which method comprises administering an effective amount of a compound of formula (I), or a pharmaceutically acceptable salt thereof, and another antiobesity agent, to a mammal in need thereof.

The invention also provides the use of a compound of formula (I), or a pharmaceutically acceptable salt thereof, and another antiobesity agent for the treatment of obesity.

The invention also provides the use of a compound of formula (I), or a pharmaceutically acceptable salt thereof, in the manufacture of a medicament for use in combination with another antiobesity agent, for the treatment of obesity.

The compound of formula (I), or a pharmaceutically acceptable salt thereof, and the other antiobesity agent(s) may be co-administered or administered sequentially or separately.

Co-administration includes administration of a formulation which includes both the compound of formula (I), or a pharmaceutically acceptable salt thereof, and the other antiobesity agent(s), or the simultaneous or separate administration of different formulations of each agent. Where the pharmacological profiles of the compound of formula (I), or a pharmaceutically acceptable salt thereof, and the other antiobesity agent(s) allow it, coadministration of the two agents may be preferred.

The invention also provides the use of a compound of formula (I), or a pharmaceutically acceptable salt thereof, and another antiobesity agent in the manufacture of a medicament for the treatment of obesity.

The invention also provides a pharmaceutical composition comprising a compound of formula (I), or a pharmaceutically acceptable salt thereof, and another antiobesity agent, and a pharmaceutically acceptable carrier. The invention also encompasses the use of such compositions in the methods described above.

GPR119 agonists are of particular use in combination with centrally acting antiobesity agents.

The other antiobesity agent for use in the combination therapies according to this aspect of the invention is preferably a CB-1 modulator, e.g. a CB-1 antagonist or inverse agonist. Examples of CB-1 modulators include SR141716 (rimonabant) and SLV-319 ((4S)-(−)-3-(4-chlorophenyl)N-methyl-N-[(4-chlorophenyl)sulfonyl]-4-phenyl-4,5-dihydro-1H-pyrazole-1-carboxamide); as well as those compounds disclosed in EP576357, EP656354, WO 03/018060, WO 03/020217, WO 03/020314, WO 03/026647, WO 03/026648, WO 03/027076, WO 03/040105, WO 03/051850, WO 03/051851, WO 03/053431, WO 03/063781, WO 03/075660, WO 03/077847, WO 03/078413, WO 03/082190, WO 03/082191, WO 03/082833, WO 03/084930, WO 03/084943, WO 03/086288, WO 03/087037, WO 03/088968, WO 04/012671, WO 04/013120, WO 04/026301, WO 04/029204, WO 04/034968, WO 04/035566, WO 04/037823 WO 04/052864, WO 04/058145, WO 04/058255, WO 04/060870, WO 04/060888, WO 04/069837, WO 04/069837, WO 04/072076, WO 04/072077, WO 04/078261 and WO 04/108728, and the references disclosed therein.

Other diseases or conditions in which GPR119 has been suggested to play a role include those described in WO 00/50562 and U.S. Pat. No. 6,468,756, for example cardiovascular disorders, hypertension, respiratory disorders, gestational abnormalities, gastrointestinal disorders, immune disorders, musculoskeletal disorders, depression, phobias, anxiety, mood disorders and Alzheimer's disease.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Abbreviations

Boc: tert-Butoxycarbonyl; t-Bu: tert-Butyl; DCM: Dichloromethane; DMAP: 4-Dimethylaminopyridine; DMF: N,N-Dimethylformamide; h: Hour; DMSO: Dimethylsulfoxide; EDC: 1-(3-Dimethylaminopropyl)-3-ethylcarbodiimide hydrochloride; EtOAc: Ethyl acetate; HOBt: 1-Hydroxybenzotriazole hydrate; HPLC: High performance liquid chromatography; mCPBA: 3-Chloroperoxybenzoic acid; IH: Isohexane; Me: Methyl; min: Minutes; RP-HPLC: Reverse phase high performance liquid chromatography; rt: Room temperature; RT: Retention time; TFA: Trifluoroacetic acid; THF: Tetrahydrofuran.

### LCMS Method 1

LCMS data were obtained as follows: Waters Xterra MS C18, 5 μm (4.6×50 mm, flow rate 1.5 mL/min) eluting with a H₂O-MeCN gradient containing 0.1% v/v ammonia over 12 min with UV detection at 215 and 254 nm. Gradient information: 0.0-8.0 min: Ramp from 95% H₂O-5% MeCN to 5% H₂O-95% MeCN; 8.0-9.9 min: Hold at 5% H₂O-95% MeCN; 9.9-10.0 min: Return to 95% H₂O-5% MeCN; 10.0-12.0 min: Hold at 95% H₂O-5% MeCN. Mass spectra were obtained using an electrospray ionization source in either the positive (ESI⁺) or negative (ESI⁻) mode.

### LCMS Method 2

LCMS data were obtained as follows: Waters Atlantis C18, 3μ (3.0×20 mm, flow rate 0.85 mL/min) eluting with a H₂O-MeCN gradient containing 0.1% v/v HCO₂H over 6.5 min with UV detection at 220 nm. Gradient information: 0.0-0.3 min 100% H₂O; 0.3-4.25 min: Ramp to 10% H₂O-90% CH₃CN; 4.25-4.4 min: Ramp to 100% CH₃CN; 4.4-4.9 min: Hold at 100% MeCN; 4.9-5.0 min: Return to 100% H₂O; 5.00-6.50 min: Hold at 100% H₂O. The mass spectra were obtained using an electrospray ionisation source in either the positive (ESI⁺) ion or negative ion (ESI⁻) mode. ¹H nmr spectra were recorded on a Varian Mercury 400 spectrometer, operating at 400 MHz. Chemical shifts are reported as ppm relative to tetramethylsilane (δ=0).

HPLC was performed using a Phenomenex™ C₁₈ column (210×21 mm) eluting with a H₂O—CH₃CN solution at 20 mL/min, with UV detection at 220 nm. Typical gradient: 0-0.5 min, 10% CH₃CN-90% H₂O; 0.5-10 min, ramp to 90% CH₃CN-10% H₂O and hold at 90% CH₃CN-10% H₂O for 5 min; 15-16 min, return to 10% CH₃CN-90% H₂O.

3-Hydroxymethylazetidine-1-carboxylic acid tert-butyl ester: Slusarchyk S. A., et al, Bioorg. Med. Chem. Lett., 2002, 12, 3235-3238; (4-Cyanophenyl)carbamic acid tert-butyl ester: Sendzik M., et al, Tetrahedron Lett. 2003, 44, 8697-8700.

### Preparation 1: 4-Carboxymethoxypiperidine-1-carboxylic acid tert-butyl ester

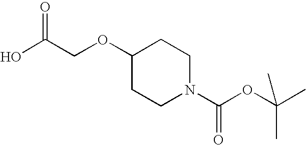

Sodium hydride (596 mg of a 60% dispersion in oil, 14.9 mmol) was added portionwise to a stirred solution of tert-butyl-4-hydroxypiperidine-1-carboxylate (1.0 g, 5 mmol) in anhydrous THF (20 mL) at rt. After 15 min bromoacetic acid (1.38 g, 9.94 mmol) was introduced and stirring continued for 5 h. Additional bromoacetic acid (5 mmol) and sodium hydride (5 mmol) were added and stirring continued for 24 h. The reaction was quenched with water (2 mL) and diluted with EtOAc (20 mL), which was washed with saturated aqueous NaHCO₃ (20 mL). The aqueous phase was acidified to pH 2 using dilute HCl and the precipitate extracted into EtOAc (50 mL), the organic phase dried (MgSO₄), evaporated and the residue purified by flash chromatography (5% AcOH in IH-EtOAc, 7:3 to 1:1) to afford the title acid: RT=2.89 min; m/z (ES⁺)=260.3 [M+H]⁺.

### Preparation 2: 3-Carboxymethoxymethylazetidine-1-carboxylic acid tert-butyl ester

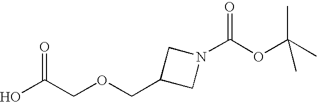

Anhydrous DMF (5 mL) was added slowly to an ice-cooled mixture of sodium hydride (120 mg of a 60% dispersion in oil, 3.2 mmol) and 3-hydroxymethylazetidine-1 carboxylic acid tert-butyl ester (400 mg, 2.13 mmol) under argon. After stirring for 15 min, solid sodium iodoacetate (666 mg, 320 mmol) was added in one portion and the stirring continued for 48 h. The resulting mixture was partitioned between water (15 mL) and EtOAc (15 mL) and the organic phase extracted further with saturated aqueous Na₂CO₃ (2×10 mL). The combined aqueous phases were acidified to pH 2 with conc HCl and extracted with EtOAc (2×60 mL). The organics were washed with brine (20 mL), dried (MgSO₄) and evaporated to give the title acid. δ_{H} (CDCl₃) 1.44 (s, 9H), 2.81 (m, 1H), 3.71 (d, 2H), 3.73 (dd, 2H), 4.02 (t, 2H), 4.13 (s, 2H).

### Preparation 3: 5-Methanesulfonylpyridine-2-carboxylic acid ethyl ester

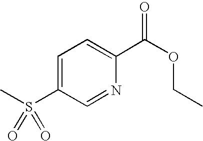

Pd(OAc)₂ (50 mg) was added to a stirred solution of 2-bromo-5-methanesulfonylpyridine (200 mg, 960 μmol) and NEt₃ (334 μL, 2A mmol) in anhydrous DMF (1.6 mL). Ethanol (570 μL) and 1,3-bis(diphenylphosphino)propane (39 mg, 95 μmol) were introduced and the resulting mixture heated at 80° C. under an atmosphere of carbon monoxide for 72 h. Following removal of the solvent, the residue was partitioned between EtOAc (50 mL) and brine (10 mL) and the organic phase separated and dried (MgSO₄). Removal of the solvent and purification of the residue by column chromatography (IH-EtOAc 4:1) afforded the title ester: δ_{H} (CDCl₃) 1.48 (3H, t), 3.15 (3H, s), 4.54 (2H, q), 8.33 (1H, d), 8.40 (1H, dd), 9.28 (1H, d).

### Preparation 4: 1-(4-Cyclopropylsulfanylphenyl)ethanone

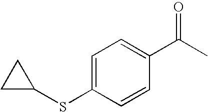

A stirred solution of aluminium trichloride (1.3 g, 9.74 mmol) in anhydrous CH₂Cl₂ (30 mL) was cooled to 0° C. and acetyl chloride (544 μL, 7.65 mmol) added, ensuring the temperature did not exceed 10° C. throughout. After stirring for 0.5 h cyclopropylsulfanylbenzene (1 mL, 6.96 mmol) and further acetyl chloride (544 μL) were added and the resulting mixture stirred for 18 h, again maintaining the temperature below 10° C. The reaction mixture was poured on to ice (40 g) and extracted with EtOAc (2×80 mL). The combined organics were washed with water (30 mL), saturated aqueous NaHCO₃ (30 mL) and brine (30 mL) then dried (MgSO₄). Removal of the solvent and purification of the residue by column chromatography (IH) afforded the title ketone: δ_{H} (CDCl₃) 0.73 (2H, m), 1.16 (2H, m), 2.21 (1H, m), 2.58 (3H, s), 7.42 (2H, d), 7.88 (2H, d).

### Preparation 5: 4-Cyclopropylsulfanylbenzoic acid

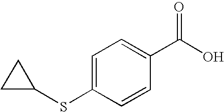

A mixture of 1-(4-cyclopropylsulfanylphenyl)ethanone (991 mg, 5.15 mmol) and pulverized KOH (2.89 g, 51.5 mmol) in DMF (120 mL) was heated at 65° C. After 18 h the solvent was removed, water (40 mL) added and the aqueous phase washed with ether (40 mL) before being acidified to pH 2 using conc HCl. The mixture was extracted into EtOAc (5×30 mL) and the combined organics dried (MgSO₄). After removal of the solvent, the residue was purified by column chromatography (DCM+10% MeOH+1% AcOH) to afford the title acid: δ_{H} (CDCl₃) 0.74 (2H, m), 1.17 (2H, m), 2.22 (1H, m), 7.43 (2H, d), 8.01 (2H, d).

### Preparation 6: 4-Methoxymethylsulfanylbenzoic acid methyl ester

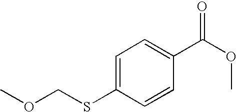

A stirred solution of 4-mercaptobenzoic acid methyl ester (500 mg, 2.97 mmol) and NEt₃ (600 μL, 4.31 mmol) in anhydrous THF (5.5 mL) was cooled to 0° C. and treated with methoxymethyl chloride (271 μL, 3.57 mmol). After 36 h the reaction was quenched with saturated aqueous NH₄Cl and extracted with ether (2×50 mL). The combined organics were washed with saturated aqueous NaHCO₃ (20 mL) and brine (10 mL) then dried (MgSO₄) and evaporated. The residue was purified by column chromatography (IH-Et₂O 9:1) to afford the title thioether: δ_{H} (CDCl₃) 3.45 (3H, s), 3.91 (3H, s), 5.05 (2H, s), 7.50 (2H, d), 7.95 (2H, d).

### Preparation 7: 4-(2-Methoxyethylsulfanyl)benzoic acid methyl ester

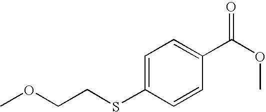

4-Mercaptobenzoic acid methyl ester was reacted with methoxyethyl chloride, using a method similar to that described in Preparation 6, to afford the title compound δ_{H} (CDCl₃) 3.20 (2H, t), 3.39 (3H, s), 3.64 (2H, t), 3.91 (3H, s), 7.34 (2H, d), 7.94 (2H, d).

### Preparation 8: trans-N-Hydroxy-4-pentylcyclohexanecarboxamidine

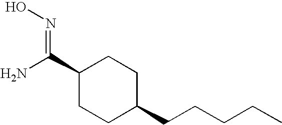

A solution of potassium carbonate (2.49 g, 18 mmol) and NH₂OH.HCl (2.50 g, 36 mmol) in water (15 mL) was added to trans-4-pentylcyclohexanecarbonitrile (4.30 g, 24 mmol) and the mixture heated to 80° C. Sufficient ethanol (approx. 45 mL) was then added to give a homogeneous solution. After 10 h, the solution was cooled, diluted with water (200 mL) and the solid material collected by filtration. The solid was dissolved in EtOAc (150 mL) and the resulting solution washed with brine (50 mL), dried (MgSO₄) and evaporated to a 15 mL volume. Hexane (60 mL) was added to precipitate the title compound: RT=2.86 min, m/z (ES⁺)=213.2 [M+H]⁺.

### Preparation 9: 4-Carbamoylmethoxypiperidine-1-carboxylic acid tert-butyl ester

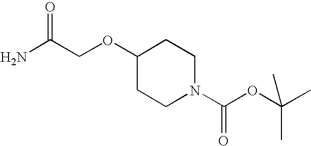

A solution of 4-carboxymethoxypiperidine-1-carboxylic acid tert-butyl ester (Preparation 1, 14.13 g, 54.7 mmol) and NEt₃ (7.68 mL, 65.6 mmol) in anhydrous THF (250 mL) was cooled to 0° C. and isobutylchloroformate (8.51 mL, 65.6 mmol) introduced dropwise. After stirring at 0° C. for 30 min, the reaction mixture was cooled to −20° C. and added rapidly via cannula to a solution of 0.7 M NH₃ in anhydrous DCM (250 mL, 180 mmol) at −70° C. The reaction was allowed to warm to rt and stirred for 1 h. The mixture was diluted with DCM (250 mL), washed with saturated aqueous NaHCO₃ (200 mL), 0.5 M aqueous HCl (200 mL) and brine (200 mL) then dried (MgSO₄). The solvent was evaporated and the residue purified by flash chromatography (1H-THF 3:7) to afford the title compound: δ_{H} (CDCl₃) 1.49 (9H, s), 1.53-1.60 (2H, m), 1.85-1.92 (2H, m), 3.11 (2H, m), 3.58 (1H, m), 3.76-3.83 (2H, m), 3.98 (2H, s), 6.19 (1H, s), 6.56 (1H, s).

### Preparation 10: 4-Cyanomethoxypiperidine-1-carboxylic acid tert-butyl ester

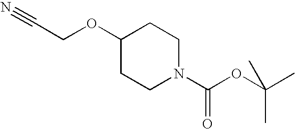

A solution of 4-carbamoylmethoxypiperidine-1-carboxylic acid tert-butyl ester (Preparation 9, 235 mg, 910 μmol) and NEt₃ (140 μL, 1 mmol) in anhydrous DCM (5 mL) was cooled to 0° C. and a solution of trichloroacetyl chloride (174 mg, 960 μmol) in anhydrous DCM (1 mL) added dropwise. The reaction mixture was stirred at rt for 1 h, the solvent removed and the residue purified by flash chromatography (IH-EtOAc 1:1) to afford the title compound: 8H (CDCl₃) 1.50 (9H, s), 1.58-1.65 (2H, m), 1.89-1.95 (2H, m), 3.20 (2H, m), 3.74-3.79 (3H, m), 4.33 (2H, s).

### Preparation 11: 4-N-Hydroxycarbamimidoylmethoxy)piperidine-1-carboxylic acid tert-butyl ester

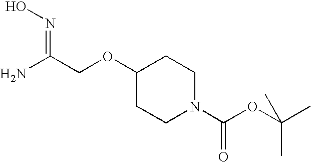

A solution of potassium carbonate (119 mg, 860 μmol) and NH₂OH.HCl (119 mg, 1.71 mmol) in water (0.5 mL) was added to 4-cyanomethoxypiperidine-1-carboxylic acid tert-butyl ester (Preparation 10, 206 mg, 857 μmol) in ethanol (2 mL). The mixture was heated at 75° C. for 0.75 h, cooled and the ethanol evaporated. The residue was diluted with EtOAc (50 mL) and washed with water (2×10 mL) and brine (10 mL) then dried (MgSO₄). The solvent was removed to afford the title compound: δ_{H} (CDCl₃) 1.50 (9H, s), 1.50-1.60 (2H, m), 1.85-1.92 (2H, m), 3.13 (2H, m), 3.56 (1H, m), 3.77-3.84 (2H, m), 4.05 (2H, s), 4.82 (2H, s); RT=2.70 min, m/z (ES⁺)=274.0 [M+H]⁺.

### Preparation 12: 2-Fluoro-4-methanesulfonylbenzoic acid

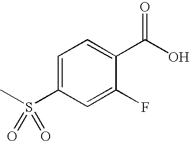

A mixture of 3-fluoro-4-methylphenylamine (3.67 g, 29.3 mmol), dimethyl disulfide (39.6 mL, 440 mmol) and tert-butyl nitrite (4.70 mL, 39.2 mmol) in 1,2-dichloroethane (400 mL) was heated at 60° C. for 10 min. A solution of 3-fluoro-4-methylphenylamine (33.0 g, 264 mmol) in 1,2-dichloroethane (100 mL) was added dropwise, whilst simultaneously adding tert-butyl nitrite (38.0 mL, 317 mmol) and maintaining the temperature at around 60° C. Following addition of reactants, the heat source was removed and the reaction stirred for 1 h. Water (200 mL) was added and the reaction mixture stirred vigorously for 10 min. The organic phase was separated and washed with water (100 mL) and 1 M aqueous HCl (200 mL) then dried (MgSO₄) and evaporated. 2-Fluoro-1-methyl-4-methylsulfanylbenzene was isolated by distillation (50° C./0.9 Torr) and used immediately as follows: a mixture of the thioether (10.0 g, 64.1 mmol) in water (200 mL) was heated to 100° C. with vigorous stirring. Potassium permanganate (45.6 g, 289 mmol) was introduced portionwise over 20 min and heating continued for 35 min then filtered through a sinter. The filtrate was cooled and extracted with EtOAc (3×200 mL), the aqueous phase acidified to pH 1 using conc HCl and extracted with EtOAc (4×150 mL). The combined organics were washed with brine (100 mL), dried (MgSO₄) and concentrated in vacuo. Et₂O was added and the precipitate collected by filtration and air-dried to give the title compound. δ_{H} (CD₃OD) 3.19 (3H, s), 7.81-7.86 (2H, m), 8.14-8.18 (1H, m).

### Preparation 13: 2-Fluoro-4-methanesulfonylbenzamide

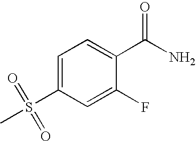

Dry NEt₃ (4.71 mL, 33.8 mmol) was added to a stirred solution of 2-fluoro-4-methanesulfonylbenzoic acid (4.91 g, 22.5 mmol) in anhydrous THF (200 mL) and the solution cooled to 0° C. Neat isobutylchloroformate (3.50 mL, 27.0 mmol) was added via syringe over 10 min and the reaction mixture warmed to rt. After 1.25 h DCM (200 mL) was added and the vessel was cooled to −78° C., ammonia (ca. 3 L) was condensed into the reaction mixture and the stirring continued for 20 min. On warming to rt, the solution was diluted with DCM (200 mL) and washed with saturated aqueous NaHCO₃ (200 mL). The aqueous phase was separated and extracted with DCM (3×100 mL). The combined organics were washed with brine (100 mL), dried (MgSO₄) and evaporated to give the title compound: δ_{H} (CD₃OD) 3.19 (3H, s), 7.81-7.88 (2H, m), 7.96-8.00 (1H, m).

### Preparation 14: 2-Fluoro-4-methanesulfonylbenzonitrile

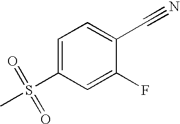

A stirred suspension of 2-fluoro-4-methanesulfonylbenzamide (3.50 g, 16.1 mmol) and NEt₃ (2.81 mL, 20.1 mmol) in DCM (100 mL) at 0° C. was treated with trichloroacetyl chloride (2.16 mL, 19.4 mmol) via syringe. After warming to rt and stirring for 2 h the reaction mixture was diluted with DCM (50 mL) and washed with saturated aqueous NaHCO₃ (100 mL). The aqueous layer was separated and extracted with DCM (100 mL) and the combined organics washed with brine (100 mL), dried (MgSO₄) and evaporated. The residue was purified by flash chromatography (EtOAc-1H 45:55) to afford the title compound δ_{H} (CDCl₃) 3.12 (3H, s), 7.84-7.92 (3H, m).

### Preparation 15: 4-Amino-2-fluorobenzonitrile

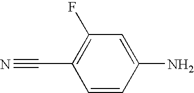

Saturated aqueous ammonium chloride (50 mL) and acetic acid (3 mL) were added to a stirred solution of 2-fluoro-4-nitrobenzonitrile (50.0 g, 301 mmol) in EtOH (600 mL) followed by iron powder (2 g, 35.7 mmol). The mixture was heated under reflux and more iron powder (123 g, 2.20 mol) added portionwise over a 4 h period. The reaction heated for a further 1 h then allowed to cool to rt before being filtered through a celite plug. The filtrate was evaporated to dryness and the residual material partitioned between EtOAc (500 mL) and water (200 mL). The organic phase was washed with water (50 mL) and brine (100 mL), then dried (MgSO₄) and evaporated to afford the title compound: δ_{H} (d₆-DMSO) 6.41-6.46 (2H, m), 6.52 (2H, br s), 7.37-7.41 (1H, m).

### Preparation 16: 3,5-Difluoro-4-methylsulfanylbenzonitrile

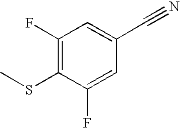

A stirred solution of 3,4,5-trifluorobenzaldehyde (6.89 g, 43 mmol) in anhydrous THF (100 mL) was cooled to 0° C. and sodium thiomethoxide (3.26 g, 46 mmol) added in small portions over 20 min. The mixture was brought to rt and stirred for 18 h then poured into water and extracted with DCM (3×200 mL). After washing with brine (50 mL), the combined organics were dried (MgSO₄) and evaporated. The residue was purified by column chromatography (IH-EtOAc-DCM 93:5:2) to give 3,5-difluoro-4-methylsulfanylbenzaldehyde: δ_{H} (CDCl₃) 2.54 (3H, s), 7.35 (2H, d), 9.86 (1H, t). A stirred solution of this aldehyde (1.0 g, 5.32 mmol) in EtOH (12 mL) was treated firstly with a solution of NH₂OH.HCl (778 mg, 11.2 mmol) in water (5 mL) followed by a solution of K₂CO₃ (780 mg, 5.64 mmol) in water (10 mL). After 1 h the EtOH was removed in-vacuo and the remaining aqueous phase extracted with EtOAc (100 mL). The organic phase was dried (MgSO₄), evaporated and the residue purified by column chromatography (IH-EtOAc 9:1) to afford 3,5-difluoro-4-methylsulfanylbenzaldehyde oxime: δ_{H} (CDCl₃) 2.50 (3H, s), 7.14 (2H, d), 7.47 (1H, s) and 8.03 (1H, s). A sample of this oxime (1.14 g, 5.6 mmol), p-toluenesulfonic acid (1.07 g, 5.6 mmol) and acetic anhydride (45 mL) was heated under reflux for 20 h. On cooling, the solvent was evaporated, and aqueous Na₂CO₃ (10 mL) added, ensuring the aqueous was pH 8. The mixture was extracted with DCM (2×100 mL) and the combined organic layers dried (MgSO₄), evaporated and the residue purified by column chromatography (IH-EtOAc 9:1) to afford the title nitrile: δ_{H} (CDCl₃) 2.58 (3H, s), 7.20 (2H, d).

### Preparation 17: 2-Fluoro-4-methylsulfanylbenzonitrile

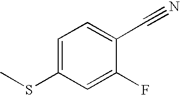

A mixture of 4-amino-2-fluorobenzonitrile (4.00 g, 29.4 mmol), dimethyl disulfide (40.6 mL, 452 mmol) and tert-butyl nitrite (4.80 mL, 40.1 mmol) in 1,2-dichloroethane (800 mL) was heated to 60° C. for 10 min. A suspension of 4-amino-2-fluorobenzonitrile (37.0 g, 272 mmol) in 1,2-dichloroethane (250 mL) was added dropwise, whilst simultaneously adding tert-butyl nitrite (43.3 mL, 362 mmol), keeping the temperature at about 60° C. After addition of reactants, the oil bath was removed and the reaction stirred for 2 h. Water (200 mL) was added and the reaction mixture stirred vigorously for 10 min. The layers were separated, the organic phase washed with water (100 mL), 2 M aqueous HCl (200 mL), brine (100 mL) then dried (MgSO₄), filtered and concentrated under reduced pressure. The residue was adsorbed onto silica gel then purified via column chromatography (EtOAc-1H, 1:9) to afford the title compound: δ_{H} (CDCl₃) 2.52 (3H, s), 6.99-7.07 (2H, m), 7.46-7.49 (1H, m).

### Preparation 18: 4-Cyano-2-fluorobenzoic acid methyl ester

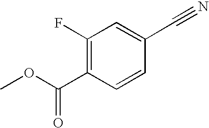

A suspension of 4-cyano-2-fluorobenzoic acid (2 g, 12.11 mmol) was suspended in toluene (5 mL) and sufficient MeOH added (ca 10 mL) to give a clear solution. Trimethylsilyldiazomethane (7.87 mL of a 2 M solution in hexane, 15.75 mmol) was added dropwise, giving a yellow solution which was stirred a further 10 min. Acetic acid was then added dropwise until a colourless solution was obtained which was then diluted with EtOAc (50 mL). The organic solution was washed with saturated aqueous Na₂CO₃ (20 mL) and brine (20 mL) then dried (MgSO₄). Evaporation of the solvent afforded the title ester: δ_{H} (CDCl₃) 3.98 (3H, s), 7.47 (1H, dd), 7.53 (1H, dd), 8.06 (1H, t).

### Preparation 19: 4-Cyano-2-fluorobenzenesulfonamide

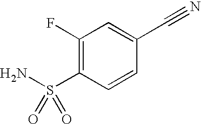

Water (15 mL) was added to a solution of 3-fluoro-4-methylsulfanylbenzonitrile (1.5 g, 8.9 μmol) in DCM (75 mL). Chlorine gas was bubbled gently through the vigorously-stirred mixture for 50 min. The organic phase was separated, dried (MgSO₄) and evaporated. The residue was dissolved in thionyl chloride (25 mL) and heated at 90° C. for 6.5 h and the solvent removed to afford 4-cyano-2-fluorobenzenesulfonyl chloride: δ_{H} (CDCl₃) 7.67 (1H, d), 7.70 (1H, d), 8.14 (1H, dd). Ammonia gas was bubbled through a stirred solution of the sulfonyl chloride (80 mg, 365 μmol) for 10 min. The solvent was then removed and the residue purified by column chromatography (IH-EtOAc 2:1) to afford the title nitrile: δ_{H} (d₆-DMSO) 7.88 (1H, dd), 7.95 (2H, br s) 7.96 (1H, t), 8.11 (1H, dd).

### Preparation 20: 3-Chloro-4-methylsulfanylbenzonitrile

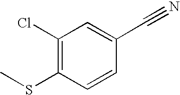

Solid sodium thiomethoxide (5.4 g, 77 mmol) was added to a stirred solution of 3-chloro-4-fluorobenzonitrile (10 g, 64 mmol) in anhydrous DMF (200 mL). The resulting mixture was heated at 80° C. for 18 h, cooled and the solvent evaporated. The residue was partitioned between EtOAc (250 mL) and water (150 mL) and the aqueous phase extracted further with EtOAc (250 mL). The combined organics were washed with water (2×100 mL) and brine (100 mL) then dried (MgSO₄) and evaporated to afford the title nitrile: δ_{H} (CDCl₃) 2.53 (3H, s), 7.20 (1H, d), 7.52 (1H, dd), 7.60 (1H, d).

The compounds listed in Table 1 were prepared by reaction of a thiolate with the appropriate 4-fluorobenzonitrile, using the method outlined in Preparation 20.

TABLE 1

| Prep | Structure | Name | δ_{H} (CDCl₃) |
|---|---|---|---|
| 21 | 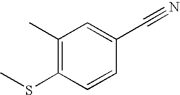 | 3-Methyl-4- methylsulfanylbenzonitrile | 2.33 (3H, s), 2.52 (3H, s), 7.16 (1H, d), 7.38 (1H, s), 7.47 (1H, d) |
| 22 | 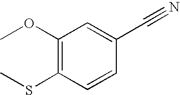 | 3-Methoxy-4- methylsulfanylbenzonitrile | 2.47 (3H, s), 3.93 (3H, s), 7.01 (1H, s), 7.14 (1H, d), 7.19-7.29 (1H, complex) |
| 23 | 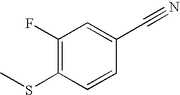 | 3-Fluoro-4-methylsulfanyl benzonitrile | 2.53 (3H, s), 7.25 (1H, d), 7.29 (1H, d), 7.43 (1H, d) |
| 24 | 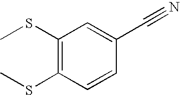 | 3,4-Bis-methylsulfanyl benzonitrile | 2.53 (3H, s), 2.54 (3H, s), 7.18 (1H, d), 7.41 (1H, d), 7.43 (1H, dd) |
| 25 | 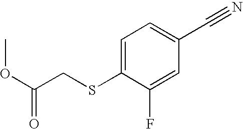 | (4-Cyano-2-fluorophenyl sulfanyl)acetic acid methyl ester | 3.75 (2H, s), 3.75 (3H, s), 7.34 (1H, dd), 7.42 (1H, dd), 7.48 (1H, t) |
| 26 | 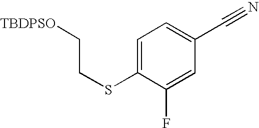 | 3-Fluoro-4-(2-tert-butyl diphenylsilyloxyethyl sulfanyl)benzonitrile | 1.05 (9H, s), 3.14 (2H, t), 3.87 (2H, t), 7.09 (1H, t), 7.23 (2H, m), 7.38- 7.46 (6H, m), 7.66 (4H, m) |

The amidoximes listed in Table 2 were prepared by reacting the corresponding nitrile with hydroxylamine, using the method outlined in Preparation 11.

TABLE 2

| Prep | Structure | Name | δ_{H} |
|---|---|---|---|
| 27 | 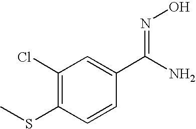 | 3-Chloro-N-hydroxy-4- methylsulfanylbenzamidine | (CDCl₃) 2.50 (3H, s), 4.86 (2H, s), 7.16 (1H, d), 7.51 (1H, d), 7.53 (1H, s), 8.05 (1H, br s) |
| 28 | 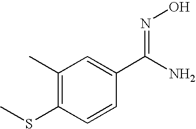 | N-Hydroxy-3-methyl-4- methylsulfanylbenzamidine | (CDCl₃) 2.35 (3H, s), 2.50 (3H, s), 4.83 (2H, s), 6.83 (1H, s), 7.16 (1H, d), 7.42 (1H, s), 7.45 (1H, d) |
| 29 | 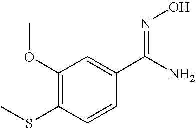 | N-Hydroxy-3-methoxy-4- methylsulfanylbenzamidine | (CDCl₃) 2.46 (3H, s), 3.94 (3H, s), 4.85 (2H, s), 7.13-7.21 (3H, m) |
| 30 | 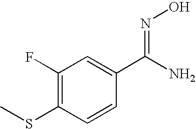 | 3-Fluoro-N-hydroxy-4- methylsulfanylbenzamidine | (CDCl₃) 2.50 (3H, s), 4.82 (2H, s), 7.18 (1H, s), 7.26 (1H, t), 7.33 (1H, dd), 7.38 (1H, dd) |
| 31 | 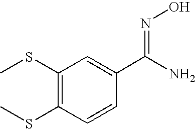 | N-Hydroxy-3,4-bis- methylsulfanylbenzamidine | (CDCl₃) 2.51 (3H, s), 2.52 (3H, s), 4.83 (2H, s), 6.89 (1H, s), 7.20 (1H, d), 7.40 (1H, dd), 7.50 (1H, d) |
| 32 | 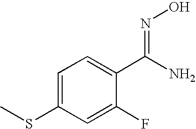 | 2-Fluoro-N-hydroxy-4- methylsulfanylbenzamidine | (d₆ DMSO) 2.52 (3H, s), 5.76 (2H, s), 7.09 (1H, dd), 7.15 (1H, dd), 7.44 (1H, t), 9.62 (1H, s) |
| 33 | 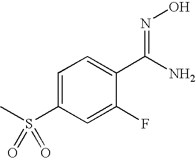 | 2-Fluoro-N-hydroxy-4- methylsulfanylbenzamidine | (d₆ DMSO) 3.29 (3H, s), 5.97 (2H, s), 7.77-7.83 (3H, m), 9.90 (1H, s) |
| 34 | 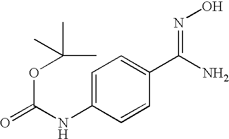 | [4-(N-Hydroxy carbamimidoyl)phenyl] carbamic acid tert-butyl ester | (CD₃OD) 1.53 (9H, s), 7.43 (2H, d), 7.55 (2H, d) |
| 35 | 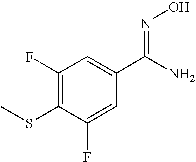 | 3,5-Difluoro-N-hydroxy-4- methylsulfanylbenzamidine | (CDCl₃) 2.50 (3H, s), 4.79 (2H, br s), 6.64 (1H, s), 7.20 (2H, d) |
| 36 | 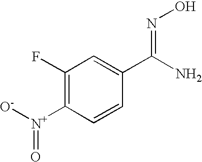 | 3-Fluoro-N-hydroxy-4- nitrobenzamide | (CDCl₃) 4.86 (2H, br s), 6.74 (1H, s), 7.59 (2H, m), 8.11 (1H, t) |
| 37 | 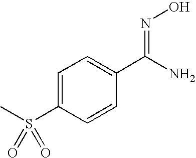 | N-Hydroxy-4-methane sulfonylbenzamide | (d₆ DMSO) 3.22 (3H, s), 5.99 (2H, s), 7.92 (4H, s), 9.97 (1H, s) |
| 38 | 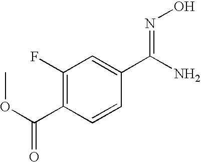 | 2-Fluora-4-(N- hydroxycarbamimidoyl)- benzoic acid methyl ester | (d₆ DMSO) 3.86 (3H, s), 6.00 (2H, s), 7.59 (1H, dd), 7.65 (1H, dd), 7.88 (1H, t), 10.04 (1H, s) |
| 39 | 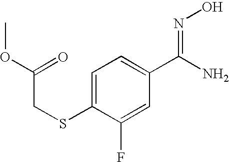 | [2-Fluoro-4-(N-hydroxy carbamimidoyl)phenyl sulfanyl]acetic acid methyl ester | (CDCl₃) 3.67 (2H, s), 3.72 (3H, s), 4.84 (2H, br s), 7.37 (2H, d), 7.43 (1H, s), 7.47 (1H, t) |
| 40 | 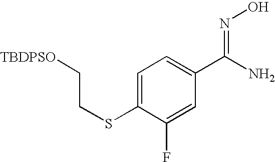 | 3-Fluoro-N-hydroxy-4-(2- tert-butyldiphenylsilyloxy ethylsulfanyl)benzamide | (CDCl₃) 1.05 (9H, s), 3.10 (2H, t), 3.84 (2H, t), 4.78 (2H, s), 6.77 (1H, s), 7.18 (1H, t), 7.24 (1H, dd), 7.29 (1H, dd), 7.36-7.46 (6H, m), 7.64-7.67 (4H, m) |
| 41 | 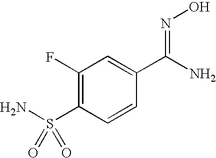 | 3-Fluoro-N-hydroxy-4- sulfamoylbenzamidine | (d₆ DMSO) 6.00 (2H, s), 7.62- 7.69 (4H, m) 7.77 (1H, t), 10.0 (1H, s) |

### Preparation 42:1-Azido-4-methanesulfonylbenzene

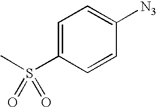

A stirred solution of 4-methanesulfonylphenylamine (1.0 g, 5.84 mmol) in TFA (1 mL) was cooled on an ice-water bath and a solution of sodium nitrite (3.1 g, 36.5 mmol) in cold water (10 mL) was added dropwise followed by a solution of sodium azide (3.25 g, 50 mmol) in water (10 mL). The reaction was allowed to warm to rt and, after 30 min, was neutralized by the portionwise addition of solid NaHCO₃. The mixture was extracted with EtOAc (100 mL), dried (MgSO₄) and evaporated to afford the title compound: δ_{H} (CDCl₃) 3.08 (3H, s), 7.21 (2H, d), 7.98 (2H, d).

### Preparation 43: 4-Bromomethyl-1-(4-methanesulfonylphenyl)-1H-[1,2,3]triazole

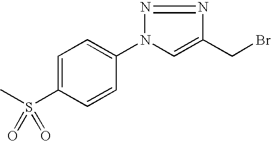

A stirred solution of 1-azido-4-methanesulfonylbenzene (1.35 g, 6.85 mmol) and prop-2-yn-1-ol (280 mg, 5 mmol) in a mixture of toluene (50 mL) and THF (50 mL) was heated at 100° C. for 17 h. After cooling, the solvent was removed and the residue purified by column chromatography (EtOAc) to afford an inseparable mixture of [1-(4-methanesulfonylphenyl)-1H-[1,2,3]triazol-4-yl]methanol and [3-(4-methanesulfonylphenyl)-3H-[1,2,3]triazol-4-yl]methanol: RT=2.32 min; m/z (ES⁺)=254.0 [M+H]⁺. A sample of this mixture of alcohols (277 mg, 1.03 mmol) was dissolved in DCM (5 mL) and thionyl chloride (600 μL, 8.22 mmol) added. After stirring for 17 h the solvent was removed and the residue purified by column chromatography (IH-EtOAc 1:1) to afford a mixture of 4-chloromethyl-1-(4-methanesulfonylphenyl)-1H-[1,2,3]triazole and 5-chloromethyl-1-(4-methanesulfonylphenyl)-1H-[1,2,3]triazole: RT=2.72 min; m/z (ES⁺)=272.0 [M+H]⁺. A solution of this mixture of chlorides (430 mg, 1.58 mmol), LiBr (1.41 g, 15.8 mmol) and acetone (10 mL) was heated under reflux for 3 h. The solvent was then removed and the residue taken up in DCM (20 mL) and washed with water (10 mL). The organic phase was passed through a hydrophobic frit, evaporated and the residue purified by column chromatography (IH-EtOAc 7:3) to give the title bromide: RT 2.65 min (method 2); m/z (ES⁺)=318.0 [M+H]⁺.

### Preparation 44: 4-Bromomethyl-1-(4-methanesulfonylphenyl)-1H-pyrazole

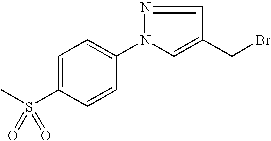

A solution of ethyl 4-pyrazolecarboxylate (102 mg, 730 μmol) in anhydrous DMSO (2 mL) was treated with sodium hydride (30 mg of a 60% dispersion in oil, 750 μmol). After stirring for 30 min, 1-fluoro-4-methanesulfonylbenzene (127 mg, 730 μmol) was added and the resulting mixture heated at 160° C. for 18 h. The cooled mixture was then poured into water (5 mL) and the precipitate collected by filtration and air-dried to give 1-(4-methanesulfonylphenyl)-1H-pyrazol 4 carboxylic acid ethyl ester RT=3.09 min; m/z (ES⁺)=295.1 [M+H]⁺. A sample of this ester (810 mg, 2.76 mmol) was dissolved in anhydrous THF (10 mL), cooled to 0° C. under argon and LiAlH₄ (3 mL of a 1M solution in THF, 3 mmol) added. After stirring for 1 h, the reaction was quenched by the addition of THF (20 mL) containing water (1 mL) and diluted with aqueous Rochelle's salts (0.5 M, 10 mL). The mixture was extracted with EtOAc (2×100 mL), the combined organics dried (MgSO₄) and evaporated to afford [1-(4-methanesulfonylphenyl)-1H-pyrazol-4-yl]methanol: RT=2.30 min; m/z (ES⁺)=253.0 [M+H]⁺. A sample of this alcohol (570 mg, 2.25 mmol) and DMF (10 μL) in dry DCM (15 mL) was treated with thionyl chloride (370 μL, 5.0 mmol). After stirring for 17 h the solvent was removed and the residue purified by column chromatography (IH-EtOAc 1:1) to afford 4-chloromethyl-1-(4-methanesulfonylphenyl)-1H-pyrazole: RT=3.01 min; m/z (ES⁺)=271.0 [M+H]⁺. A mixture of this chloride (212 mg, 780 μmol) and LiBr (347 mg, 3.9 mmol) in acetone (50 mL) was heated under reflux for 3 h, cooled and the solvent removed. The residue was taken up in CH₂Cl₂ (20 mL), washed with water (10 mL) and the organic phase dried by passage through a hydrophobic frit. Evaporation afforded the title bromide: RT=3.09 min (method 2); m/z (ES⁺)=315.0 [M+H]⁺.

### Preparation 45: 2,5-Difluoro-4-methylsulfanylbenzoic acid

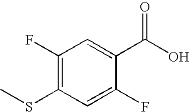

A methanolic (5 mL) solution of 2,4,5-trifluorobenzoic acid (2 g, 11.4 mmol) was diluted with toluene (15 mL) and trimethylsilyldiazomethane (6.8 mL of a 2 M solution in hexane, 13.7 mmol) was added dropwise. After 5 min, 100 μL of glacial acetic acid was added and the mixture diluted with EtOAc (100 mL), washed with water and brine then dried (MgSO₄). Evaporation of the solvent afforded 2,4,5-trifluorobenzoic acid methyl ester: 8H (CDCl₃) 3.94 (3H, s), 7.01 (1H, dt), 7.80 (1H, ddd). A stirred solution of this ester 1.0 g, 5.7 mmol) in anhydrous DMF (15 mL) was cooled to −78° C. and a suspension of sodium thiomethoxide (402 mg, 5.7 mmol) in anhydrous DMF was added. The mixture was allowed to warm to rt over 2.5 h and poured into water (25 mL). After extraction into EtOAc (150 mL) the organic phase was washed with water (20 mL), dried (MgSO₄) and evaporated to dryness. Purification of the residue by column chromatography (IH-DCM 7:3) afforded 2,5-difluoro-4-methylsulfanylbenzoic acid methyl ester: δ_{H} (CDCl₃) 2.50 (3H, s), 3.92 (3H, s), 6.91 (1H, dd), 7.57 (1H, dd). A solution of this ester (300 mg, 1.4 mmol) in MeOH (15 mL) was treated with LiOH (645 mg, 15 mmol) and the mixture stirred for 2 h at rt. After acidification to pH 1 using conc HCl, the methanol was evaporated to a small volume and the title acid collected by filtration: δ_{H} (CDCl₃) 2.48 (3H, s), 6.89 (1H, dd), 7.57 (1H, dd).

### Preparation 46: 4-(2-Carboxyethoxy)piperidine-1-carboxylic acid tert-butyl ester

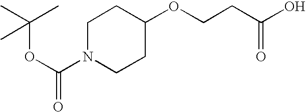

Solid 4-hydroxypiperidine-1-carboxylic acid tert-butyl ester (3 g, 14.9 mmol) and sodium hydride (42 mg of a 60% dispersion in oil, 1 mmol) were combined in a flask which was flushed with argon. Methyl acrylate (13.4 mL, 149 mmol) was added and the mixture stirred at rt for 18 h. Residual solvent was removed in vacuo and the residue purified by column chromatography (IH-EtOAc 9:1 then 1:1) to afford 4-(2-methoxycarbonylethoxy)piperidine-1-carboxylic acid tert-butyl ester: δ_{H} (CDCl₃) 1.46 (9H, s), 1.52 (2H, m), 1.81 (2H, m), 2.59 (2H, t), 3.12 (2H, ddd), 3.48 (1H, dddd), 3.70 (3H, s), 3.72 (2H, m), 3.74 (2H, t). A sample of this ester (933 mg, 3.25 mmol) was dissolved in MeOH (10 mL) and aqueous NaOH (3.25 mL of a 2 M solution, 6.5 mmol) was added. After stirring for 1 h, the solvent was evaporated, the residue dissolved in water (50 mL) and washed with ether (20 mL). The aqueous phase was acidified to pH 2 using dilute HCl and extracted into EtOAc (100 mL). Drying of the organic phase (MgSO₄) and removal of the solvent afforded the title acid: δ_{H} (CDCl₃) 1.46 (9H, s), 1.54 (2H, m), 1.82 (2H, m), 2.64 (2H, t), 3.12 (2H, ddd), 3.51 (1H, dddd), 3.72 (2H, m), 3.75 (2H, t).

### Preparation 47: 5-Chloro-3-(4-methanesulfonylphenyl)-[1,2,4]oxadiazole

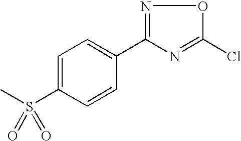

A stirred solution of N-hydroxy-4-methanesulfonylbenzamidine (2.14 g, 10 mmol) and pyridine (1 mL) in anhydrous DMF (15 mL) was cooled to 0° C. and neat methyl chloroformate (0.85 mL, 11 mmol) was added in one portion. The mixture was warmed to rt and after 1.5 h the solvent was evaporated and water (20 mL) and 2% MeOH in EtOAc (150 mL) added. The aqueous was separated and extracted again with 2% MeOH in EtOAc. The combined organics were washed with brine and dried (MgSO₄). The solvent was removed and the residue dissolved in pyridine (10 mL). After heating under reflux for 3 h the pyridine was evaporated to afford 3-(4-methanesulfonylphenyl)-[1,2,4]oxadiazol-5-ol: δ_{H} (d₆-DMSO) 3.29 (3H, s), 8.06 (2H, d), 8.12 (2H, d). A sample of 3-(4-methanesulfonylphenyl)-[1,2,4]oxadiazol-5-ol (1.23 g, 5.15 mmol) dissolved in POCl₃ (30 mL) was heated under gentle reflux and pyridine (0.42 mL, 5.12 mmol) and 4-DMAP (4 mg) added. Heating was continued for 22 h whereupon the mixture was allowed to cool and poured onto ice/water 300 (mL). The aqueous was extracted with EtOAc (3×150 mL), the combined organics washed with brine, dried (MgSO₄) and evaporated to afford the title chlorooxadiazole: δ_{H} (d₆-DMSO) 3.30 (3H, s), 8.14 (2H, d), 8.24 (2H, d).

### Preparation 48: 4-(2-Ethoxycarbonyl-1-hydroxyethyl)piperidine-1-carboxylic acid tert-butyl ester

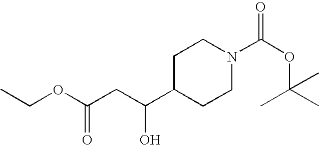

Solid sodium borohydride (374 mg, 9.88 mmol) was added to a solution of 4-(2-ethoxycarbonylacetyl)piperidine-1-carboxylic acid tert-butyl ester (2.69 g, 8.98 mmol) in EtOH (80 mL). After stirring at rt for 0.5 h, the solvent was evaporated and the residue purified by column chromatography (IH-EtOAc) to give the title ester: δ_{H} (CDCl₃) 1.25 (2H, m), 1.29 (3H, t), 1.46 (9H, s), 1.52 (1H, m), 1.60 (1H, m), 1.85 (1H, dddd), 2.42 (11, dd), 2.53 (1H, dd), 2.67 (2H, br t), 3.02 (1H, d), 3.79 (1H, m), 4.16 (2H, m), 4.19 (2H, q).

### Preparation 49: 3-Fluoro-4-methanesulfinylbenzoic acid and 3-fluoro-methylsulfonylbenzoic acid

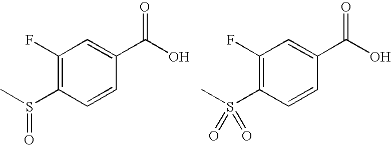

A stirred solution of 3,4-difluorobenzaldehyde (5.0 g, 35.2 mmol) in dry acetonitrile (50 mL) under argon was treated portionwise with sodium thiomethoxide (2.47 g, 35.2 mmol) over approximately 1 h. After 18 h the reaction mixture was diluted with EtOAc (50 mL) and washed with saturated aqueous NaHCO₃ (2×10 mL) and saturated aqueous NH₄Cl (20 mL). The organic phase was dried (MgSO₄), evaporated and the residue purified by column chromatography (IH-EtOAc 9:1 then 7:3) to give 3-fluoro-4-methylsulfanylbenzaldehyde: δ_{H} (CDCl₃) 2.54 (3H, s), 7.32 (1H, t), 7.51 (1H, dd), 7.63 (1H, dd), 9.92 (1H, d). A sample of this thioether (1.0 g, 5.38 mmol) was suspended in water and NaH₂PO₄ (705 mg, 5.88 mmol), tert-butanol (44 mL) and sodium chlorite (1.59 g, 63 mmol) were then added. After stirring vigorously for 1.5 h, the tert-butanol was removed under reduced pressure and EtOAc (50 mL) added. The mixture was extracted with 1 M aqueous NaOH (3×20 mL) and the combined extracts acidified to pH 2 using dilute HCl. The precipitate was extracted into EtOAc which was dried (MgSO₄) and evaporated to afford an inseparable mixture of the title sulfoxide: RT=2.22 min (method 2); m/z (ES⁺)=203.0 [M+H]⁺ and the sulfone 2.22 min (method 2); m/z (ES⁺)=219.0 [M+H]⁺.

### General Procedures for the Synthesis of Oxadiazoles

### Method A:

### 4-[5-(3-Cyano-4-methoxyphenyl)-[1,2,4]oxadizol-3-ylmethoxy]piperidine-1-carboxylic acid tert-butyl ester

### Example 1

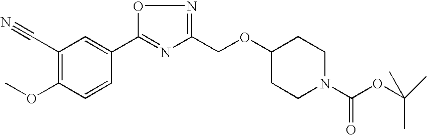

A mixture of 4-N-hydroxycarbamimidoylmethoxy)piperidine-1-carboxylic acid tert-butyl ester (Preparation 11, 150 mg, 0.36 mmol) and KOtBu (49 mg, 0.44 mmol) in DMSO (1 mL) was sonicated and heated until a workable solution was obtained, which was then added to 3-cyano-4-methoxybenzoic acid methyl ester (71 mg, 0.4 mmol). The reaction mixture was stirred at 60° C. for 15 h, neutralised with acetic acid (5 drops) and purified by RP-HPLC to afford the title compound. δ_{H} (CDCl₃) 1.49 (9H, s), 1.68 (2H, m), 1.95 (2H, m), 3.12 (2H, m), 3.72 (1H, m), 3.84 (2H, m), 4.08 (3H, s), 4.75 (2H, s), 7.17 (1H, d), 8.36 (1H, dd), 8.41 (1H, d).

### Method B:

### 4-[5-(4-Methanesulfonylphenyl)-[1,2,4]oxadizol-3-ylmethoxy]piperidine-1-carboxylic acid tert-butyl ester

### Example 2

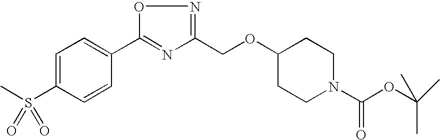

A solution of 4-methanesulfonylbenzoic acid (88 mg, 0.44 mmol) and HOBt (60 mg, 0.44 mmol) in THF (0.8 mL) was treated with EDC (76 mg, 0.44 mmol) and subsequently a suspension of 4-(N-hydroxycarbamrimidoylmethoxy)piperidine-1-carboxylic acid tert-butyl ester (Preparation 11, 109 mg, 0.4 mmol) in THF (0.4 mL). The mixture was stirred at rt overnight.

A solution of KO^{t}Bu (205 mg, 1.83 mmol) in THF (0.6 mL) was added, causing formation of a thick, solid precipitate. Sufficient THF (0.3 mL) was added to give a fluid mixture, which was stirred for a further 2 h. The solvent was evaporated, the residue dissolved in a mixture of DMSO (0.3 mL), MeOH (0.4 mL) and DCM (0.4 mL) and purified by RP-HPLC to afford the title compound: δ_{H} (CDCl₃) 1.49 (9H, s), 1.68 (2H, m), 1.95 (2H, m), 3.15 (3H, s), 3.16 (2H, m), 3.75 (1H, m), 3.84 (2H, m), 4.80 (2H, s), 8.17 (2H, d), 8.41 (2H, d).

### Method C:

### 4-[5-(2-Fluoro-4-methanesulfonylphenyl)-[1,2,4]oxadizol-3-ylmethoxy]piperidine-1-carboxylic acid tert-butyl ester

### Example 3

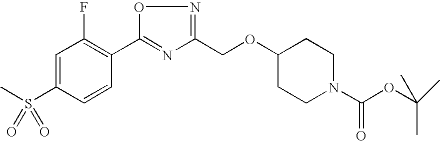

A stirred solution of 2-fluoro-4-methanesulfonylbenzoic acid (100 mg, 460 μmol) and triethylamine (64 μL, 460 μmol) in dry THF (5 mL) was cooled in an ice-water bath and isobutylchloroformate (60 μL, 460 μmol) added. The reaction mixture was warmed to rt and, after 20 min, 4-(N-hydroxycarbamimidoylmethoxy)piperidine-1-carboxylic acid tert-butyl ester (Preparation 11, 104 mg, 380 μmol) was added and the mixture allowed to stir overnight. The reaction mixture was diluted with EtOAc (20 mL), washed with water (20 mL), saturated aqueous NaHCO₃ (20 mL), brine (20 mL) and dried (MgSO₄). The solvent was removed and the residue dissolved in toluene (15 mL), 4A molecular sieves (0.25 g) were added and the mixture heated under reflux for 4 days. On cooling the solution was filtered through Celite, evaporated and the residue purified by column chromatography, affording the title compound: δ_{H} (CDCl₃) 1.47 (9H, s), 1.64 (2H, m), 1.91 (2H, m), 3.13 (2H, ddd), 3.14 (3H, s), 3.72 (1H, tt), 3.81 (2H, m), 4.79 (2H, s), 7.89 (1H, d), 7.91 (1H, d), 8.41 (1H, t); RT=3.65 min (method 2), m/z (ES⁺)=456.1 [M+H]⁺.

### Method D:

### 4-[5-(4-Methanesulfanylphenyl)-[1,2,4]oxadizol-3-ylmethoxy]piperidine-1-carboxylic acid tert-butyl ester

### Example 4

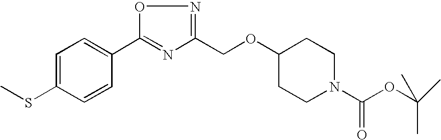

Oxalyl chloride (0.83 mL, 9.51 mmol) was added in a dropwise manner to a suspension of 4-methanesulfanylbenzoic acid (1.23 g, 7.31 mmol) in dry DCM (25 mL). After stirring at rt for 2 h, the solvent and excess oxalyl chloride were removed in vacuo. The residue was dissolved in dry DCM (30 mL) and triethylamine (2.55 mL, 18.3 mmol) added followed by a solution of 4-(N-hydroxycarbamimidoylmethoxy)piperidine-1-carboxylic acid tert-butyl ester (Preparation 11, 2.00 g, 7.32 mmol). After 3 h water (30 mL) and DCM (30 mL) were added, the organic phase separated, dried (MgSO₄) and evaporated. The residue was dissolved in anhydrous THF (20 mL) under argon and sodium hydride (287 mg, 12.0 mmol) added portion-wise. After stirring for 36 h, the solvent was removed, saturated aqueous ammonium chloride (30 mL) added, and the resulting suspension extracted into EtOAc (2×80 mL). The combined organics were washed with brine, dried (MgSO₄) and evaporated. The residue was then purified by column chromatography (IH-EtOAc 3:2) to give the title compound: δ_{H} (CDCl₃) 1.50 (9H, s), 1.65 (2H, m), 1.95 (2H, m), 2.60 (3H, s), 3.15 (2H, m), 3.75 (1H, m), 3.80 (2H, m), 4.80 (2H, s), 7.40 (2H, d), 8.05 (2H, d).

### Method E:

### 4-[3-(4-Methylsulfanylphenyl)-[1,2,4]oxadiazol-5-ylmethoxy]piperidine-1-carboxylic acid tert-butyl ester

### Example 5

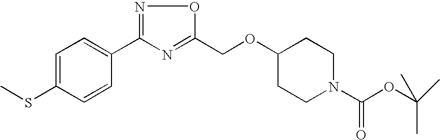

A stirred solution of 4-carboxymethoxypiperidine-1-carboxylic acid tert-butyl ester (Preparation 1, 854 mg, 2.11 mmol) and triethylamine (460 μL, 3.3 mmol) in toluene (20 mL) was cooled in an ice-water bath and isobutylchloroformate (430 μL, 3.31 mmol) added. After 10 min the reaction mixture was brought to rt and stirred a further 45 min. N-Hydroxy-4-methylsulfanylbenzamidine (500 mg, 2.74 mmol) was then added in one portion and the resulting solution heated under reflux for 18 h then cooled to rt. Saturated aqueous NaHCO₃ was added and the mixture extracted with EtOAc. The organic phase was washed with water, brine, dried (MgSO₄) and evaporated, and the residue purified by column chromatography (IH-EtOAc 17:3) to afford the title compound: δ_{H} (CDCl₃) 1.47 (9H, s), 1.64 (2H, m), 1.91 (2H, m), 2.54 (3H, s), 3.14 (2H, ddd), 3.72 (1H, tt), 3.79 (2H, m), 4.83 (2H, s), 7.33 (2H, d), 8.01 (2H, d); RT=4.20 min (method 2), m/z (ES⁺)=406.1 [M+H]⁺.

### Method F:

### 4-[5-(3-Cyan-4-fluorophenyl)-[1,2,4]oxadiazol-3-ylmethoxy]piperidine-1-carboxylic acid tert-butyl ester

### Example 6

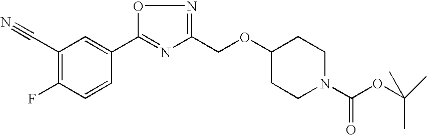

A solution comprised of 3-cyano-4-fluorobenzoic acid (200 mg, 1.21 mmol), diisopropylethylamine (695 μL, 4.0 mmol), 4-(N-hydroxycarbamimidoylmethoxy)piperidine-1-carboxylic acid tert-butyl ester (Preparation 11, 364 mg, 1.33 mmol) and HOBt (204 mg, 1.51 mmol) in dry DMF (10 mL) was stirred for 5 min and EDC (280 mg, 1.46 mmol) added. After stirring for a further 22 h, the mixture was poured into water (5 mL) and extracted with EtOAc (50 mL). The organic phase was washed with water, dried (MgSO₄), evaporated and the residue passed through a column of silica (IH-EtOAc 1:3). After removing the solvent, the solid residue was suspended in toluene and the mixture heated under reflux for 21 h. The cooled the solution was evaporated on to silica and purified by column chromatography (IH-EtOAc 7:3) to afford the title compound: δ_{H} (CDCl₃) 1.47 (9H, s), 1.63 (2H, m), 1.92 (2H, m), 3.12 (2H, ddd), 3.71 (1H, tt), 3.80 (2H, m), 4.74 (2H, s), 7.44 (1H, t), 8.41 (1H, ddd), 8.47 (1H, dd); RT=3.87 min (Method 2), m/z (ES⁺)=403.1 [M+H]⁺.

Each of the oxadiazoles listed in Table 3 were synthesised using the general method indicated.

TABLE 3

| | | | LCMS | |
|---|---|---|---|---|
| | | | RT (min) | |
| Eg | Structure | Name [Synthetic Method] | [Method] | m/z |
| 7 | 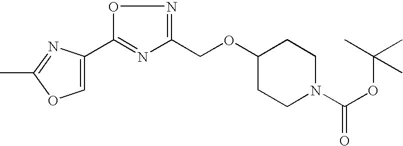 | 4-[5-(2-Methyloxazol-4-yl)- [1,2,4]oxadiazol-3-ylmethoxy] piperidine-1-carboxylic acid tert- butyl ester [A] | 5.92 [1] | 365.1 [M + H]⁺ |
| 8 | 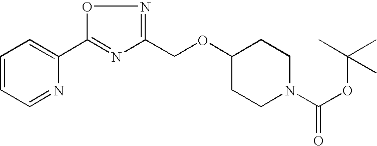 | 4-(5-Pyridin-2-yl-[1,2,4] oxadiazol-3-ylmethoxy) piperidine-1-carboxylic acid tert- butyl ester [A] | 5.97 [1] | 361.1 [M + H]⁺ |
| 9 | 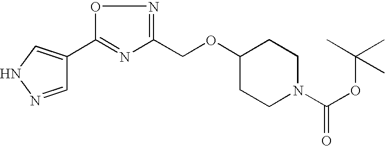 | 4-[5-(1H-Pyrazol-4-yl)-[1,2,4] oxadiazol-3-ylmethoxy] piperidine-1-carboxylic acid tert- butyl ester [A] | 5.22 [1] | 350.1 [M + H]⁺ |
| 10 | 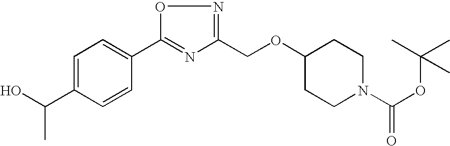 | 4-{5-[4-(1-Hydroxyethyl) phenyl]-[1,2,4]oxadiazol-3-yl methoxy}piperidine-1- carboxylic acid ten-butyl ester [A] | 6.30 [1] | 404.1 [M + H]⁺ |
| 11 | 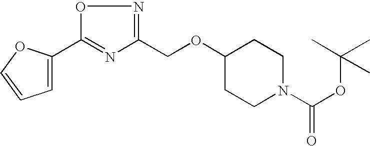 | 4-(5-Furan-2-yl-[1,2,4]oxadiazol- 3-ylmethoxy)piperidine-1- carboxylic acid tert-butyl ester [A] | 6.55 [1] | 350.1 [M + H]⁺ |
| 12 | 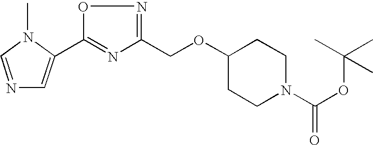 | 4-[5-(3-Methyl-3H-imidazol-4- yl)-[1,2,4]oxadiazol-3-yl methoxy]piperidine-1-carboxylic acid tert-butyl ester [A] | 5.55 [1] | 364.1 [M + H]⁺ |
| 13 | 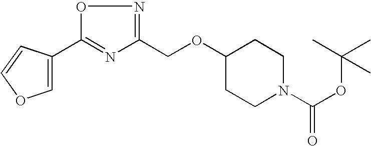 | 4-(5-Furan-3-yl[1,2,4]oxadiazol- 3-ylmethoxy)piperidine-1- carboxylic acid tert-butyl ester [A] | 6.59 [1] | 350.1 [M + H]⁺ |
| 14 | 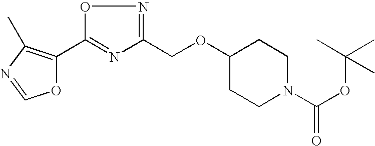 | 4-[5-(4-Methyloxazol-5-yl)- [1,2,4]oxadiazol-3-ylmethoxy] piperidine-1-carboxylic acid tert- butyl ester [A] | 6.15 [1] | 365.1 [M + H]⁺ |
| 15 | 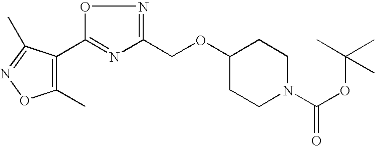 | 4-[5-(3,5-Dimethylisoxazol-4- yl)-[1,2,4]oxadiazol-3-yl methoxy]piperidine-1-carboxylic acid tert-butyl ester [A] | 6.89 [1] | 379.1 [M + H]⁺ |
| 16 | 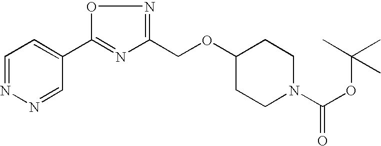 | 4-(5-Pyridazin-4-yl-[1,2,4] oxadiazol-3-ylmethoxy) piperidine-1-carboxylic acid tert- butyl ester [B] | 4.37 [1] | 2.62 [M + H − C₅H₈O₂]⁺ |
| 17 | 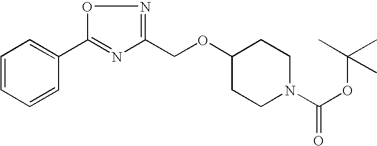 | 4-(5-Phenyl-[1,2,4]oxadiazol-3- ylmethoxy)piperidine-1- carboxylic acid tert-butyl ester [A] | 6.39 [1] | 360.1 [M + H]⁺ |
| 18 | 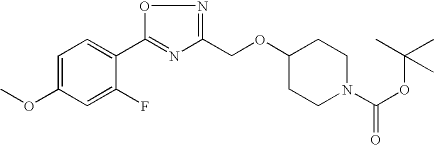 | 4-[5-(2-Fluoro-4-methoxy phenyl)-[1,2,4]oxadiazol-3-yl methoxy]piperidine-1-carboxylic acid tert-butyl ester [B] | 6.43 [1] | 408.1 [M + H]⁺ |
| 19 | 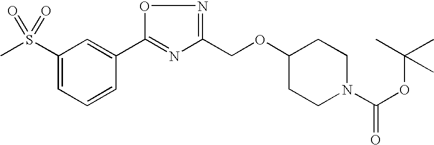 | 4-[5-(3-Methanesulfonylphenyl)- [1,2,4]oxadiazol-3-ylmethoxy] piperidine-1-carboxylic acid tert- butyl ester [B] | 5.47 [1] | 438.0 [M + H]⁺ |
| 20 | 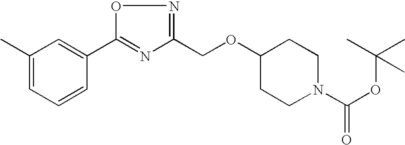 | 4-(5-m-Tolyl-[1,2,4]oxadiazol-3- ylmethoxy)piperidine-1- carboxylic acid tert-butyl ester [B] | 6.85 [1] | 374.1 [M + H]⁺ |
| 21 | 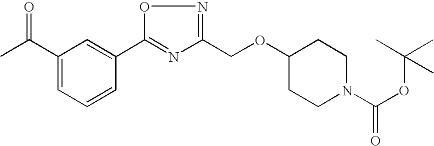 | 4-[5-(3-Acetylphenyl)-[1,2,4] oxadiazol-3-ylmethoxy] piperidine-1-carboxylic acid tert- butyl ester [B] | 6.02 [1] | 402.1 [M + H]⁺ |
| 22 | 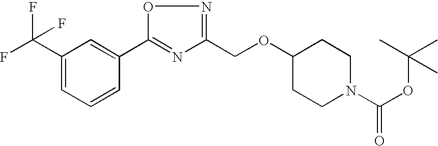 | 4-[5-(3-Trifluoromethylphenyl)- [1,2,4]oxadiazol-3-ylmethoxy] piperidine-1-carboxylic acid tert- butyl ester [B] | 7.09 [1] | 428.1 [M + H]⁺ |
| 23 | 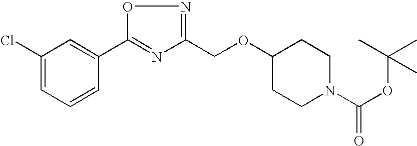 | 4-[5-(3-Chlorophenyl)- [1,2,4]oxadiazol-3-ylmethoxy] piperidine-1-carboxylic acid tert- butyl ester [B] | 7.00 [1] | 394.1 [M + H]⁺ |
| 24 | 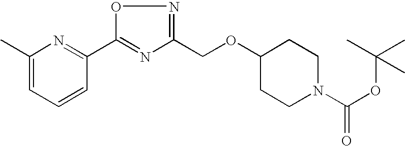 | 4-[5-(6-Methylpyridin-2-yl)- [1,2,4]oxadiazol-3-ylmethoxy] piperidine-1-carboxylic acid tert- butyl ester [B] | 5.49 [1] | 375.1 [M + H]⁺ |
| 25 | 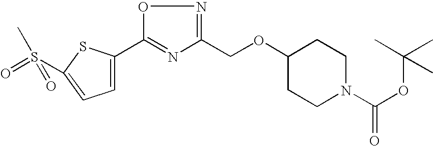 | 4-[5-(5-Methanesulfonylthiophen- 2-yl)-[1,2,4]oxadiazol-3-yl- methoxy]piperidine-1-carboxylic acid tert-butyl ester [B] | 5.62 [1] | 444.0 [M + H]⁺ |
| 26 | 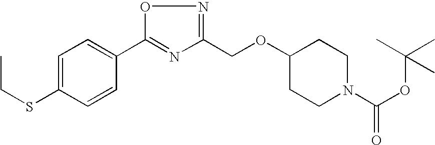 | 4-[5-(4-Ethylsulfanylphenyl)- [1,2,4]oxadiazol-3-ylmethoxy] piperidine-1-carboxylic acid tert- butyl ester [C] | 4.37 [2] | 420.1 [M + H]⁺ |
| 27 | 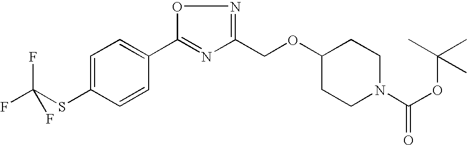 | 4-[5-(4-Trifluoromethylsulfanyl phenyl)-[1,2,4]oxadiazol-3- ylmelhoxy]piperidine-1- carboxylic acid tert-butyl ester [C] | 4.39 [2] | 460.1 [M + H]⁺ |
| 28 | 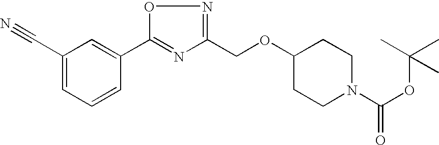 | 4-[5-(3-Cyanophenyl)-[1,2,4] oxadiazol-3-ylmethoxy] piperidine-1-carboxylic acid tert- butyl ester [C] | 3.65 [2] | 385.1 [M + H]⁺ |
| 29 | 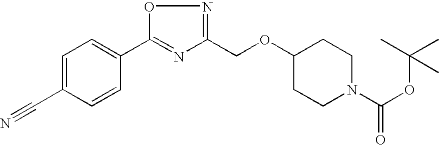 | 4-[5-(4-Cyanophenyl)-[1,2,4] oxadiazol-3-ylmethoxy] piperidine-1-carboxylic acid tert- butyl ester [C] | 3.82 [2] | 385.2 [M + H]⁺ |
| 30 | 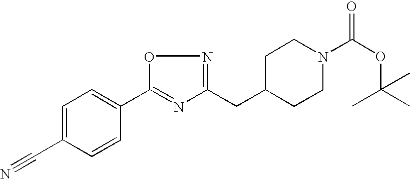 | 4-[5-(4-Cyanophenyl)-[1,2,4] oxadiazol-3-ylmethyl]piperidine- 1-carboxylic acid tert-butyl ester [C] | 3.99 [2] | 369.2 [M + H]⁺ |
| 31 | 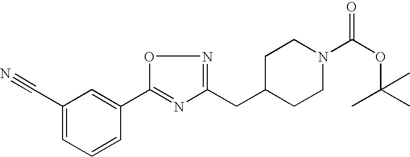 | 5-(3-Cyanophenyl)-[1,2,4] oxadiazol-3-ylmethyl]piperidine- 1-carboxylic acid tert-butyl ester [C] | 3.90 [2] | 269.1 [M + H − C₅H₈O₂]⁺ |
| 32 | 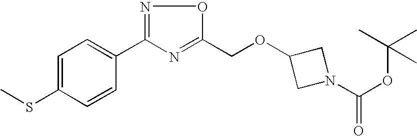 | 3-[3-(4-Methylsulfanylphenyl)- [1,2,4]oxadiazol-5-ylmethoxy] azetidine-1-carboxylic acid tert- butyl ester [C] | 3.87 [2] | 378.1 [M + H]⁺ |
| 33 | 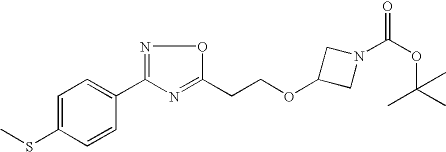 | 3-{2-[3-(4-Methylsulfanyl phenyl)-[1,2,4]oxadiazol-5- yl]ethoxy}azetidine-1-carboxylic acid tert-butyl ester [C] | 4.02 [2] | 392.2 [M + H]⁺ |
| 34 | 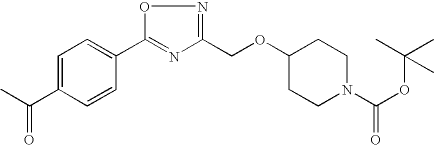 | 4-[5-(4-Acetylphenyl)-[1,2,4] oxadiazol-3-ylmethoxy] piperidine-1-carboxylic acid tert- butyl ester [B] | 3.85 [2] | 402.2 [M + H]⁺ |
| 35 | 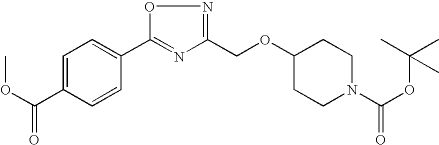 | 4-[5-(4-Methoxycarbonylphenyl)- [1,2,4]oxadiazol-3-ylmethoxy] piperidine-1-carboxylic acid tert- butyl ester [C] | 3.96 [2] | 418.2 [M + H]⁺ |
| 36 | 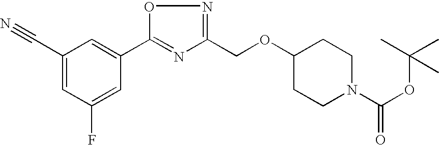 | 4-[5-(3-Cyano-5-fluorophenyl)- [1,2,4]oxadiazol-3-ylmethoxy] piperidine-1-carboxylic acid tert- butyl ester [F] | 3.94 [2] | 403.2 [M + H]⁺ |
| 37 | 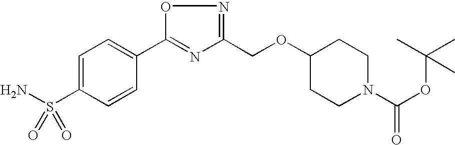 | 4-[5-(4-Sulfamoylphenyl)- [1,2,4]oxadiazol-3-ylmethoxy] piperidine-1-carboxylic acid tert- butyl ester [A] | 3.44 [2] | 439.1 [M + H]⁺ |
| 38 | 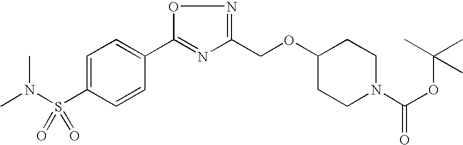 | 4-[5-(4-Dimethylsulfamoyl phenyl)-[1,2,4]oxadiazol-3-yl methoxy]piperidine-1-carboxylic acid tert-butyl ester [B] | 3.82 [2] | 467.2 [M + H]⁺ |
| 39 | 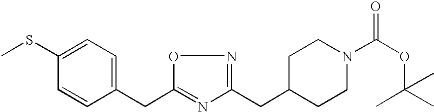 | 4-[5-(4-Methylsulfanylbenzyl)- [1,2,4]oxadiazol-3-ylmethyl] piperidine-1-carboxylic acid tert- butyl ester [B] | 4.12 [2] | 348.1 [M + H − C₄H₈]⁺ |
| 40 | 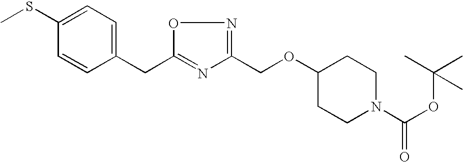 | 4-[5-(4-Methylsulfanylbenzyl)- [1,2,4]oxadiazol-3-ylmethoxy] piperidine-1-carboxylic acid tert- butyl ester [B] | 3.94 [2] | 452.2 [M + H]⁺ |
| 41 | 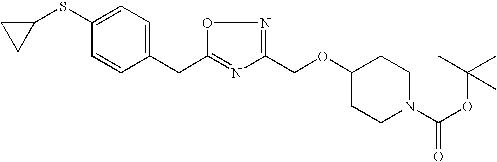 | 4-[5-(4-Cyclopropylsulfanyl benzyl)-[1,2,4]oxadiazol-3- ylmethoxy]piperidine-1- carboxylic acid tert-butyl ester [B] | 4.19 [2] | 446.2 [M + H]⁺ |
| 42 | 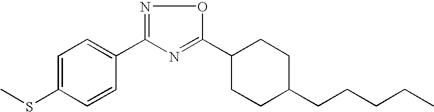 | 3-(4-Methylsulfanylphenyl)-5- (4-pentylcyclohexyl)-[1,2,4] oxadiazole [E] | 5.19 [2] | 345.3 [M + H]⁺ |
| 43 | 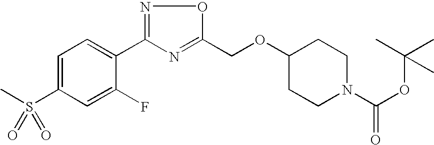 | 4[3-(2-Fluoro-4-methane sulfonylphenyl)-[1,2,4] oxadiazol-5-ylmethoxy] piperidine-1-carboxylic acid tert- butyl ester [B] | 3.69 [2] | 456.2 [M + H]⁺ |
| 44 | 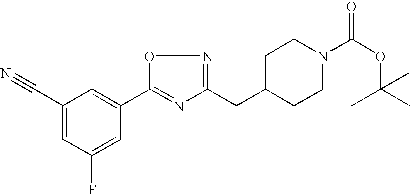 | 4-[5-(3-Cyano-5-fluorophenyl)- [1,2,4]oxadiazol-3-ylmethyl] piperidine-1-carboxylic acid tert- butyl ester [F] | 3.90 [2] | 387.2 [M + H]⁺ |
| 45 | 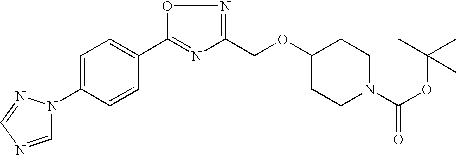 | 4-[5-(4-[1,2,4]Triazol-1-yl phenyl)-[1,2,4]oxadiazol-3-yl methoxy]piperidine-1-carboxylic acid tert-butyl ester [F] | 3.59 [2] | 427.2 [M + H]⁺ |
| 46 | 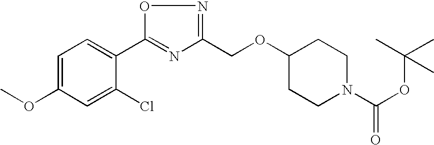 | 4-[5-(2-Chloro-4-methoxy phenyl)-[1,2,4]oxadiazol-3-yl methoxy]piperidine-1-carboxylic acid tert-butyl ester [B] | 4.11 [2] | 424.1 [M + H]⁺ |
| 47 | 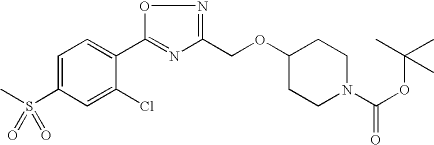 | 4-[5-(2-Chloro-4-methane sulfonylphenyl)-[1,2,4]oxadiazol- 3-ylmethoxy]piperidine-1- carboxylic acid tert-butyl ester [B] | 3.52 [2] | 472.1 [M + H]⁺ |
| 48 | 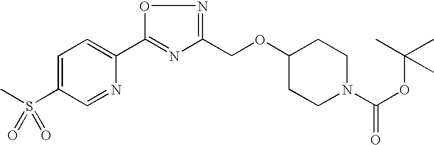 | 4-[5-(5-Methanesulfonylpyridin- 2-yl)-[1,2,4]oxadiazol-3-yl methoxy]piperidine-1-carboxylic acid tert-butyl ester [A] | 3.34 [2] | 439.2 [M + H]⁺ |
| 49 | 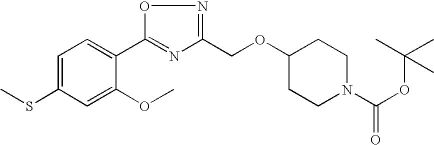 | 4-[5-(2-Methoxy-4-methyl sulfanylphenyl)-[1,2,4]oxadiazol- 3-ylmethoxy]piperidine-1- carboxylic acid tert-butyl ester [D] | 3.97 [2] | 436.2 [M + H]⁺ |
| 50 | 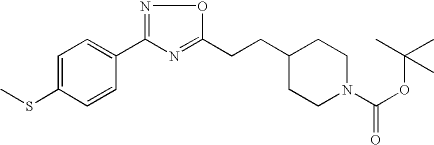 | 4-{2-[3-(4-Methylsulfanyl phenyl)-[1,2,4]oxadiazol-5-yl]- ethyl}piperidine-1-carboxylic acid tert-butyl ester [B] | 4.45 [2] | 404.2 [M + H]⁺ |
| 51 | 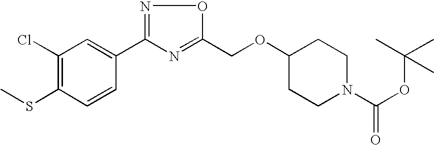 | 4-[3-(3-Chloro-4-methylsulfanyl phenyl)-[1,2,4]oxadiazol-5-yl methoxy]piperidine-1-carboxylic acid tert-butyl ester [F] | 4.39 [2] | 440.1 [M + H]⁺ |
| 52 | 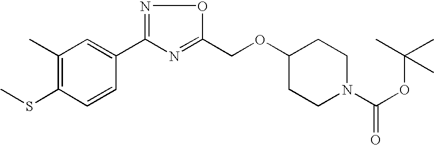 | 4-[3-(3-Methyl-4-methylsulfanyl phenyl)-[1,2,4]oxadiazol-5-yl methoxy]piperidine-1-carboxylic acid tert-butyl ester [F] | 4.29 [2] | 420.2 [M + H]⁺ |
| 53 | 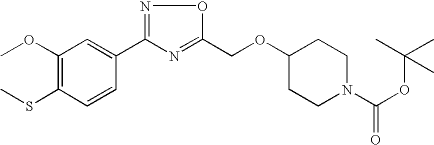 | 4-[3-(3-Methoxy-4-methyl sulfanylphenyl)-[1,2,4]oxadiazol- 5-ylmethoxy]piperidine-1- carboxylic acid tert-butyl ester [F] | 4.12 [2] | 436.2 [M + H]⁺ |
| 54 | 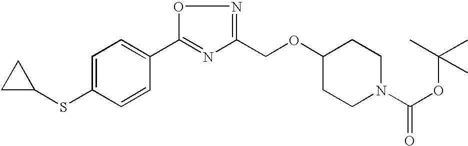 | 4-[5-(4-Cyclopropyl sulfanylphenyl)-[1,2,4] oxadiazol-3-ylmethoxy] piperidine-1-carboxylic acid tert- butyl ester [F] | 4.24 [2] | 432.2 [M + H]⁺ |
| 55 | 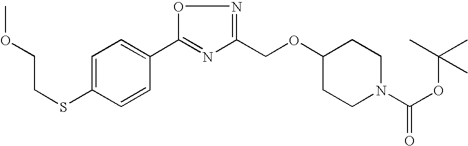 | 4-{5-[4-(2-Methoxyethyl sulfanyl)phenyl]-[1,2,4] oxadiazol-3-ylmethoxy} piperidine-1-carboxylic acid tert- butyl ester [A] | 3.92 [2] | 450.2 [M + H]⁺ |
| 56 | 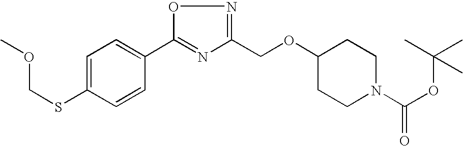 | 4-[5-(4-Methoxymethylsulfanyl phenyl)-[1,2,4]oxadiazol-3-yl methoxy]piperidine-1-carboxylic acid tert-butyl ester [A] | 4.01 [2] | 436.2 [M + H]⁺ |
| 57 | 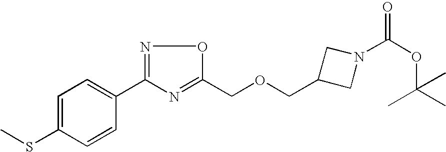 | 3-[3-(4-Methylsulfanylphenyl)- [1,2,4]oxadiazol-5-yl methoxymethyl]azetidine-1- carboxylic acid tert-butyl ester [E] | 4.02 [2] | 392.2 [M + H]⁺ |
| 58 | 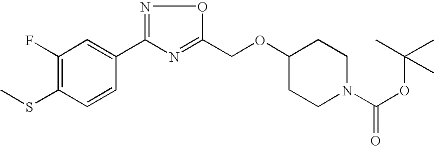 | 4-[3-(3-Fluoro-4-methylsulfanyl phenyl)-[1,2,4]oxadiazol-5- ylmethoxy]piperidine-1- carboxylic acid tert-butyl ester [E] | 4.26 [2] | 424.2 [M + H]⁺ |
| 59 | 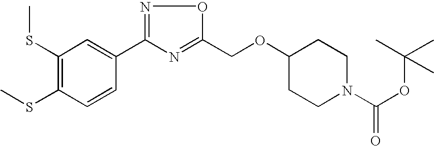 | 4-[3-(3,4-Bis-methylsulfanyl phenyl)-[1,2,4]oxadiazol-5-yl methoxy]piperidine-1-carboxylic acid tert-butyl ester [E] | 4.26 [2] | 452.2 [M + H]⁺ |
| 60 | 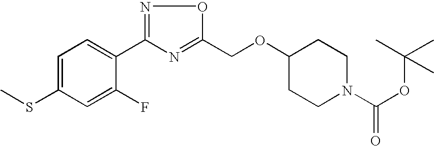 | 4-[3-(2-Fluoro-4-methylsulfanyl phenyl)-[1,2,4]oxadiazol-5-yl methoxy]piperidine-1-carboxylic acid tert-butyl ester [F] | 3.95 [2] | 424.1 [M + H]⁺ |
| 61 | 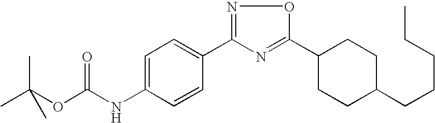 | {4-[5-(4-Pentylcyclohexyl)- [1,2,4]oxadiazol-3-yl]phenyl} carbamic acid tert-butyl ester [C] | 519 [2] | 414.3 [M + H]⁺ |
| 62 | 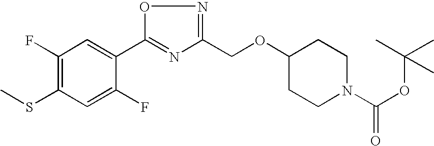 | 4-[5-(2,5-Difluoro-4-methyl sulfanylphenyl)-[1,2,4]oxadiazol- 3-ylmethoxy]piperidine-1- carboxylic acid tert-butyl ester [F] | 3.72 [2] | 442.0 [M + H]⁺ |
| 63 | 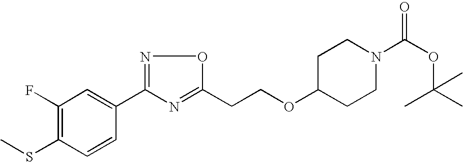 | 4-{2-[3-(3-Fluoro-4-methyl sulfanylphenyl)-[1,2,4]oxadiazol- 5-yl]ethoxy}piperidine-1- carboxylic acid tert-butyl ester [C] | 4.24 [2] | 438.1 [M + H]⁺ |
| 64 | 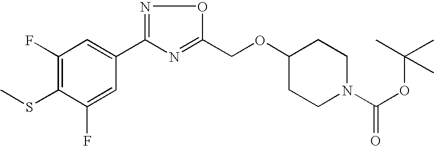 | 4-[3-(3,5-Difluoro-4-methyl sulfanylphenyl)-[1,2,4]oxadiazol- 5-ylmethoxy]piperidine-1- carboxylic acid tert-butyl ester [C] | 4.27 [2] | 442 [M + H]⁺ |
| 65 | 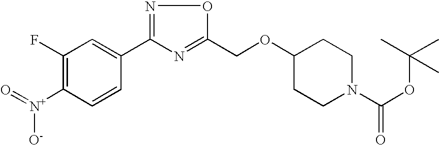 | 4-[3-(3-Fluoro-4-nitrophenyl)- [1,2,4]oxadiazol-5-ylmethoxy] piperidine-1-carboxylic acid tert- butyl ester [C] | 4.01 [2] | 423.0 [M + H]⁺ |
| 66 | 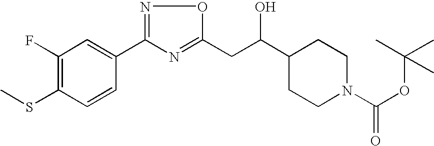 | 4-{2-[3-(3-Fluoro-4-methyl sulfanylphenyl)-[1,2,4] oxadiazol-5-yl]-1-hydroxy ethyl}piperidine-1-carboxylic acid tert-butyl ester [A] | 3.82 [2] | 438.0 [M + H]⁺ |
| 67 | 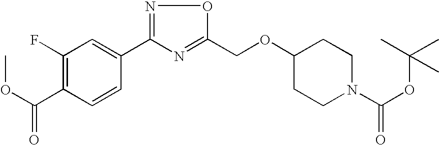 | 4-[3-(3-Fluoro-4-methoxy carbonylphenyl)-[1,2,4] oxadiazol-5-ylmethoxy] piperidine-1-carboxylic acid tert- butyl ester [F] | 4.09 [2] | 436.0 [M + H]⁺ |
| 68 | 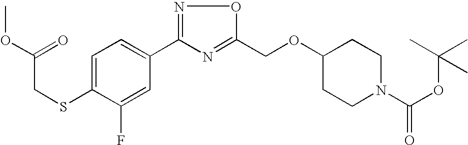 | 4-[3-(3-Fluoro-4-methoxy carbonylmethylsulfanylphenyl)- [1,2,4]oxadiazol-5-ylmethoxy] piperidine-1-carboxylic acid tert- butyl ester [C] | 4.11 [2] | 482.1 [M + H]⁺ |
| 69 | 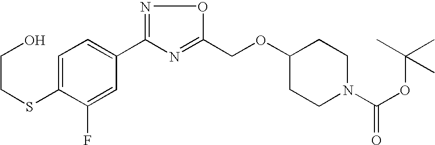 | 4-{3-[3-Fluoro-4-(2-hydroxy ethylsulfanyl)phenyl]-[1,2,4] oxadiazol-5-ylmethoxy} piperidine-1-carboxylic acid tert- butyl ester [C] | 3.31 [2] | 470.1 [M + H]⁺ |
| 70 | 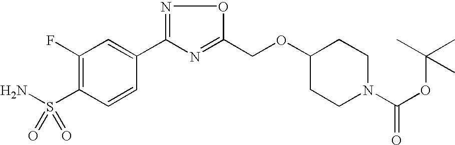 | 4-[3-(3-Fluoro-4-sulfamoyl phenyl)-[1,2,4]oxadiazol-5-yl methoxy]piperidine-1-carboxylic acid tert-butyl ester [F] | 3.49 [2] | 457.0 [M + H]⁺ |
| 71 | 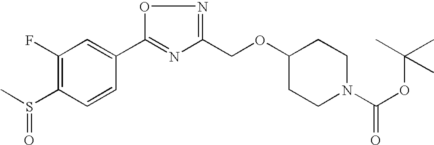 | 4-[5-(3-Fluoro-4-methane sulfinylphenyl)-[1,2,4]oxadiazol- 3-ylmethoxy]piperidine-1- carboxylic acid tert-butyl ester [F] | 3.47 [2] | 440.0 [M + H]⁺ |
| 72 | 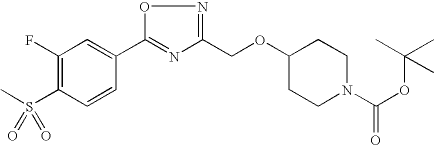 | 4-[5-(3-Fluoro-4-methane sulfonylphenyl)-[1,2,4]oxadiazol- 3-ylmethoxy]piperidine-1- carboxylic acid tert-butyl ester [F] | 3.65 [1] | 456.0 [M + H]⁺ |
| 73 | 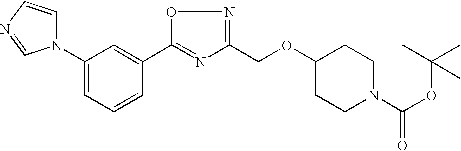 | 4-[5-(3-Imidazol-1-ylphenyl)- [1,2,4]oxadiazol-3-ylmethoxy] piperidine-1-carboxylic acid tert- butyl ester [B] | 5.70 [1] | 426.0 [M + H]⁺ |
| 74 | 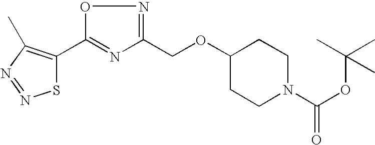 | 4-[5-(4-Methyl-[1,2,3]thiadiazol- 5-yl)-[1,2,4]oxadiazol-3-yl methoxy]piperidine-1-carboxylic acid tert-butyl ester [B] | 3.47 [1] | 382.0 [M + H]⁺ |
| 75 | 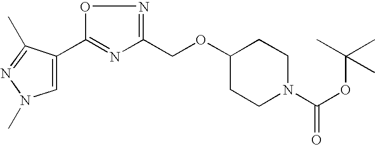 | 4-[5-(1,3-Dimethyl-1H-pyrazol- 4-yl)-[1,2,4]oxadiazol-3-yl methoxy]piperidine-1-carboxylic acid tert-butyl ester [B] | 5.42 [1] | 378.0 [M + H]⁺ |
| 76 | 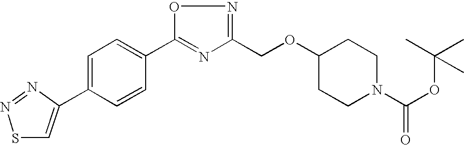 | 4-[5-(4-[1,2,3]Thiadiazol-4-yl phenyl)-[1,2,4]oxadiazol-3-yl methoxy]piperidine-1-carboxylic acid tert-butyl ester [B] | 6.85 [1] | 443.9 [M + H]⁺ |
| 77 | 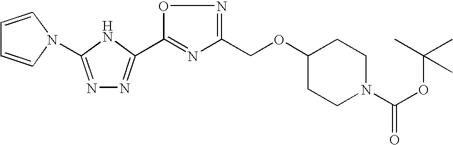 | 4-[5-(5-Pyrrol-1-yl-4H-[1,2,4] triazol-3-yl)-[1,2,4]oxadiazol-3- ylmethoxy]piperidine-1-car- boxylic acid tert-butyl ester [B] | 1.65 [1] | 416.0 [M + H]⁺ |
| 78 | 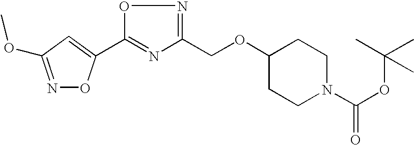 | 4-[5-(3-Methoxyisoxazol-5-yl)- [1,2,4]oxadiazol-3-ylmethoxy] piperidine-1-carboxylic acid tert- butyl ester [B] | 5.97 [1] | 381.0 [M + H]⁺ |
| 79 | 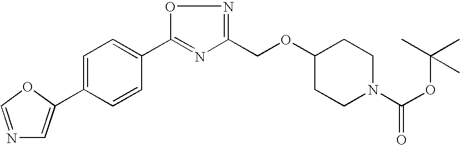 | 4-[5-(4-Oxazol-5-ylphenyl)- [1,2,4]oxadiazol-3-ylmethoxy] piperidine-1-carboxylic acid tert- butyl ester [B] | 6.14 [1] | 427.0 [M + H]⁺ |
| 80 | 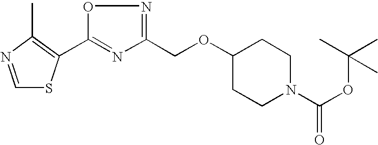 | 4-[5-(4-Methylthiazol-5-yl)- [1,2,4]oxadiazol-3-ylmethoxy] piperidine-1-carboxylic acid tert- butyl ester [B] | 3.18 [1] | 380.9 [M + H]⁺ |
| 81 | 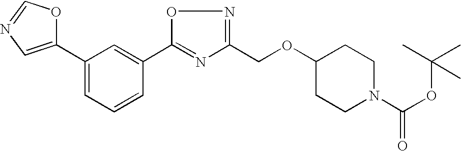 | 4-[5-(3-Oxazol-5-ylphenyl)- [1,2,4]oxadiazol-3-ylmethoxy] piperidine-1-carboxylic acid tert- butyl ester [B] | 6.15 [1] | 427.0 [M + H]⁺ |
| 82 | 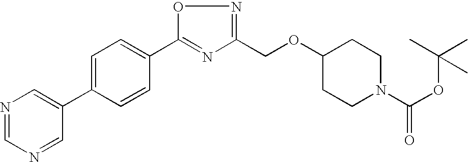 | 4-[5-(4-Pyrimidin-5-ylphenyl)- [1,2,4]oxadiazol-3-ylmethoxy] piperidine-1-carboxylic acid tert- butyl ester [B] | 5.74 [1] | 438.0 [M + H]⁺ |
| 83 | 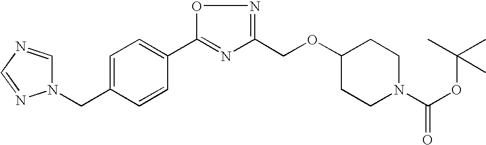 | 4-[5-(4-[1,2,4]Triazol-1-yl methylphenyl)-[1,2,4]oxadiazol- 3-ylmethoxy]piperidine-1-carboxylic acid tert-butyl ester [B] | 5.43 [1] | 441.0 [M + H]⁺ |
| 84 | 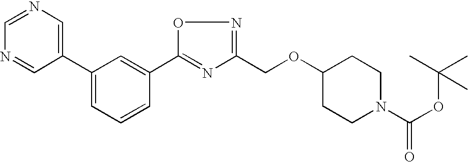 | 4-[5-(3-Pyrimidin-5-ylphenyl)- [1,2,4]oxadiazol-3-ylmethoxy] piperidine-1-carboxylic acid tert- butyl ester [B] | 5.72 [1] | 438.0 [M + H]⁺ |
| 85 | 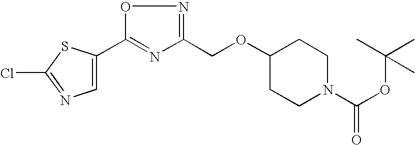 | 4-[5-(2-Chlorothiazol-5-yl)- [1,2,4]oxadiazol-3-ylmethoxy] piperidine-1-carboxylic acid tert- butyl ester [B] | 3.65 [1] | 400.9 [M + H]⁺ |
| 86 | 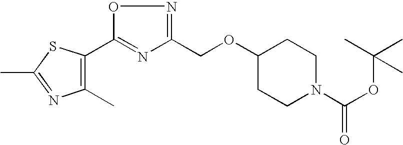 | 4-[5-(2,4-Dimethylthiazol-5-yl)- [1,2,4]oxadiazol-3-ylmethoxy] piperidine-1-carboxylic acid tert- butyl ester [B] | 5.93 [1] | 395.0 [M + H]⁺ |
| 87 | 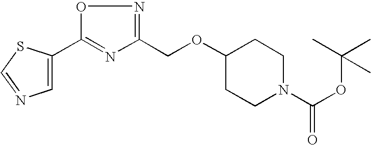 | 4-(5-Thiazol-5-yl-[1,2,4] oxadiazol-3-ylmethoxy) piperidine-1-carboxylic acid tert- butyl ester [B] | 5.43 [1] | 367.0 [M + H]⁺ |
| 88 | 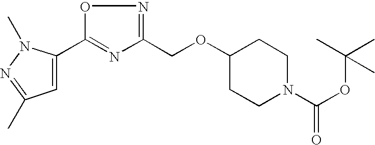 | 4-[5-(2,5-Dimethyl-2H-pyrazol- 3-yl)-[1,2,4]oxadiazol-3-yl methoxy]piperidine-1-carboxylic acid tert-butyl ester [B] | 5.85 [1] | 378.0 [M + H]⁺ |
| 89 | 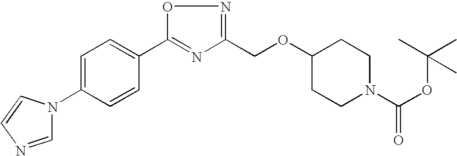 | 4-{5-(4-Imidazol-1-ylphenyl)- [1,2,4]oxadiazol-3-ylmethoxy] piperidine-1-carboxylic acid tert- butyl ester [B] | 5.70 [1] | 426.0 [M + H]⁺ |
| 90 | 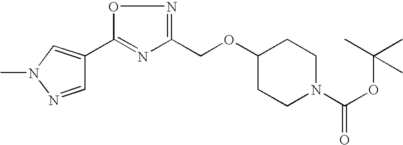 | 4-[5-(1-Methyl-1H-pyrazol-4- yl)-[1,2,4]oxadiazol-3-yl methoxy]piperidine-1-carboxylic acid tert-butyl ester [B] | 5.45 [1] | 364.0 [M + H]⁺ |
| 91 | 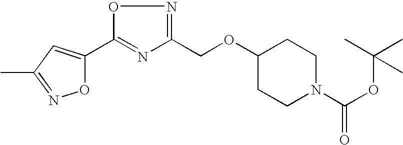 | 4-[5-(3-Methylisoxazol-5-yl)- [1,2,4]oxadiazol-3-ylmethoxy] piperidine-1-carboxylic acid tert- butyl ester [B] | 6.18 [1] | 365.0 [M + H]⁺ |
| 92 | 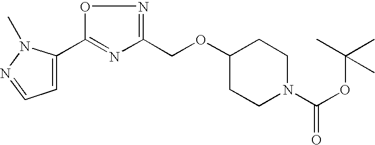 | 4-[5-(2-Methyl-2H-pyrazol-3- yl)-[1,2,4]oxadiazol-3-yl methoxy]piperidine-1-carboxylic acid tert-butyl ester [B] | 3.17 [1] | 364 [M + H]⁺ |
| 93 | 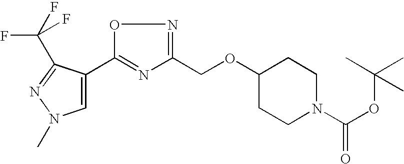 | 4-[5-(1-Methyl-3-trifluoromethyl- 1H-pyrazol-4-yl)-[1,2,4] oxadiazol-3-ylmethoxy] piperidine-1-carboxylic acid tert- butyl ester [B] | 6.12 [1] | 432.0 [M + H]⁺ |
| 94 | 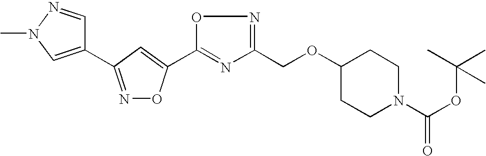 | 4-{5-[3-(1-Methyl-1H-pyrazol- 4-yl)isoxazol-5-yl]-[1,2,4] oxadiazol-3-ylmethoxy} piperidine-1-carboxylic acid tert- butyl ester [B] | 6.02 [1] | 431.0 [M + H]⁺ |
| 95 | 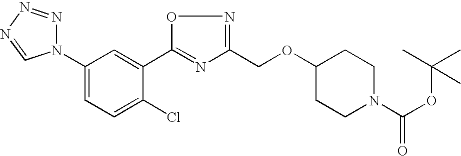 | 4-[5-(2-Chloro-5-tetrazol-1-yl phenyl)-[1,2,4]oxadiazol-3-yl methoxy]piperidine-1-carboxylic acid tert-butyl ester [B] | 3.45 [1] | 461.9 [M + H]⁺ |

### Example 96 and 97

### 4-[3-(4-Methylsulfinylphenyl)-[1,2,4]oxadiazol-5-ylmethoxy]piperidine-1-carboxylic acid tert-butyl ester and 4-[3-(4-methylsulfonylphenyl)-[1,2,4]oxadiazol-5-ylmethoxy]piperidine-1-carboxylic acid tert-butyl ester

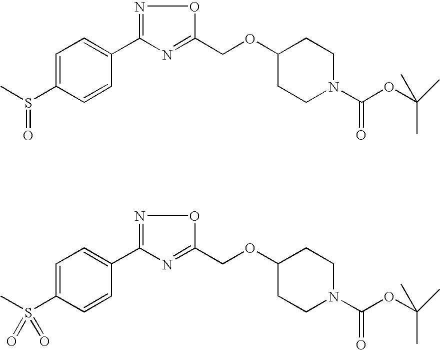

mCPBA (540 mg of 70% purity, 2.19 mmol) was added in one portion to a stirred solution of 4-[3-(4-methylsulfanylphenyl)-[1,2,4]oxadiazol-5-ylmethoxy]piperidine-1-carboxylic acid tert-butyl ester (Example 5, 590 mg, 1.45 mmol) in DCM (50 mL). After 1 h, the reaction mixture was washed with saturated aqueous NaHCO₃ (20 mL), the organic phase dried (MgSO₄) and evaporated on to silica. Purification by column chromatography, eluting firstly with IH-EtOAc 1:1, afforded the title sulfone: δ_{H} (CDCl₃) 1.50 (9H, s), 1.68 (2H, m), 1.95 (2H, m), 3.14 (3H, s), 3.18 (2H, m), 3.77 (1H, m), 3.83 (2H, m), 4.90 (2H, s), 8.12 (2H, d), 8.35 (2H, d). Further elution with neat EtOAc afforded the title sulfoxide: δ_{H} (CDCl₃) 1.49 (9H, s), 1.67 (2H, m), 1.94 (2H, m), 2.81 (3H, s), 3.18 (2H, m), 3.76 (1H, m), 3.82 (2H, m), 4.89 (2H, s), 7.81 (2H, d), 8.30 (2H, d).

Reacting the sulfide with a single equivalent of mCPBA afforded only the sulfoxide; reaction with two equivalents afforded the sulfone as the sole product.

The compounds listed in Table 4 were produced by oxidation with mCPBA using the method outlined in Example 96 & 97 and were analysed by LCMS method 2.

TABLE 4

| Eg | Structure | Name | RT (min) | m/z |
|---|---|---|---|---|
| 98 | 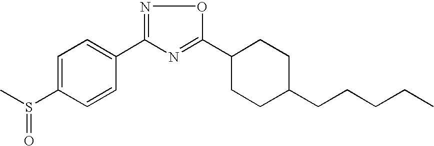 | 3-(4-Methanesulfinylphenyl)-5- (4-pentylcyclohexyl)-[1,2,4] Oxadiazole | 4.62 | 361.3 [M + H]⁺ |
| 99 | 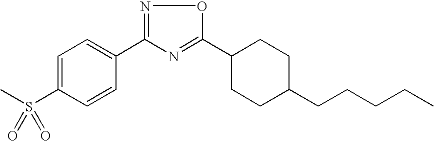 | 3-(4-Methanesulfonylphenyl)-5- (4-pentylcyclohexyl)-[1,2,4] Oxadiazole | 4.79 | 376.5 [M + H]⁺ |
| 100 | 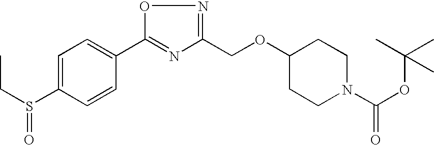 | 4-[5-(4-Ethanesulfinylphenyl)- [1,2,4]oxadiazol-3-ylmethoxy] piperidine-1-carboxylic acid tert- butyl ester | 3.49 | 436.2 [M + H]⁺ |
| 101 | 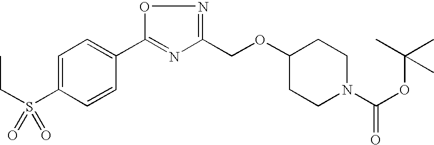 | 4-[5-(4-Ethanesulfonylphenyl)- [1,2,4]oxadiazol-3-ylmethoxy] piperidine-1-carboxylic acid tert- butyl ester | 3.67 | 452.1 [M + H]⁺ |
| 102 | 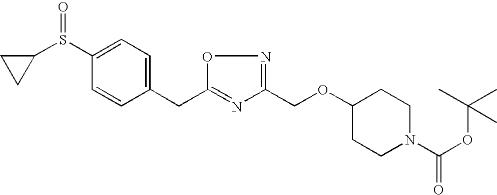 | 4-[5-(4-Cyclopropanesulfinyl benzyl)-[1,2,4]oxadiazol-3-yl methoxy]piperidine-1-carboxylic acid tert-butyl ester | 3.39 | 462.2 [M + H]⁺ |
| 103 | 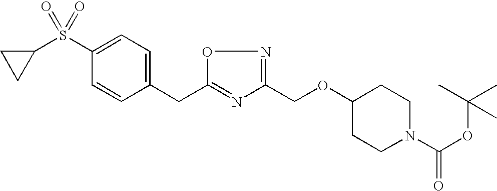 | 4-[5-(4-Cyclopropanesulfonyl benzyl)-[1,2,4]oxadiazol-3-yl methoxy]piperidine-1-carboxylic acid tert-butyl ester | 3.54 | 478.2 [M + H]⁺ |
| 104 | 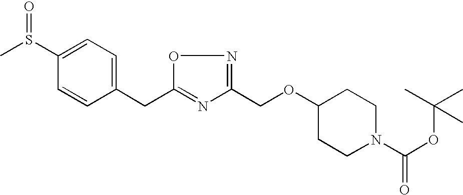 | 4-[5-Methanesulfinylbenzyl)- [1,2,4]oxadiazol-3-ylmethoxy] piperidine-1-carboxylic acid tert- butyl ester | 3.20 | 436.2 [M + H]⁺ |
| 105 | 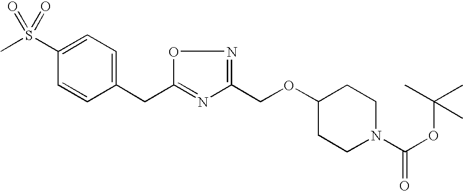 | 4-[5-(4-Methanesulfonylbenzyl)- [1,2,4]oxadiazol-3-ylmethoxy] piperidine-1-carboxylic acid tert- butyl ester | 3.39 | 452.2 [M + H]⁺ |
| 106 | 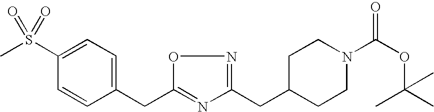 | 4-[5-(4-Methanesulfonylbenzyl)- [1,2,4]oxadiazol-3-ylmethyl] piperidine-1-carboxylic acid tert- butyl ester | 3.49 | 436.2 [M + H]⁺ |
| 107 | 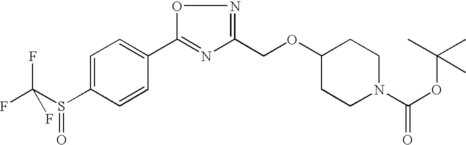 | 4-[5-(4-Trifluoromethanesulfinyl phenyl)-[1,2,4]oxadiazol-3-yl methoxy]piperidine-1-carboxylic acid tert-butyl ester | 3.90 | [M + H]⁺ |
| 108 | 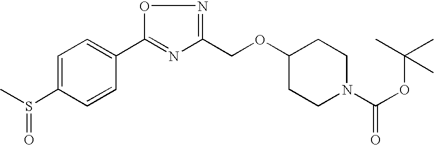 | 4-[5-(4-Methanesulfinylphenyl)- [1,2,4]oxadiazol-3-ylmethoxy] piperidine-1-carboxylic acid tert- butyl ester | 3.20 | 422.1 [M + H]⁺ |
| 109 | 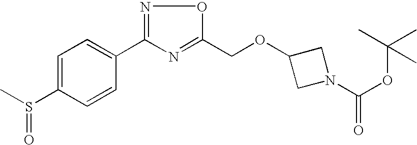 | 3-[3-(4-Methanesulfinylphenyl)- [1,2,4]oxadiazol-5-ylmethoxy] azetidine-1-carboxylic acid tert- bulyl ester | 3.19 | 394.1 [M + H]⁺ |
| 110 | 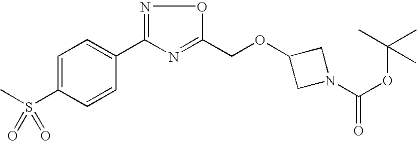 | 3-[3-(4-Methanesulfonylphenyl)- [1,2,4]oxadiazol-5-ylmethoxy] azetidine-1-carboxylic acid tert- butyl ester | 3.45 | 410.1 [M + H]⁺ |
| 111 | 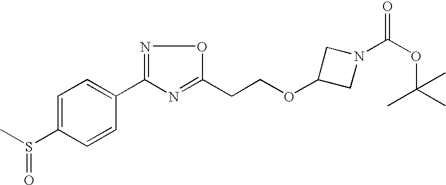 | 3-{2-[3-(4-Methanesulfinyl phenyl)-[1,2,4]oxadiazol-5-yl] ethoxy}azetidine-1-carboxylic acid tert-butyl ester | 3.24 | 408.1 [M + H]⁺ |
| 112 | 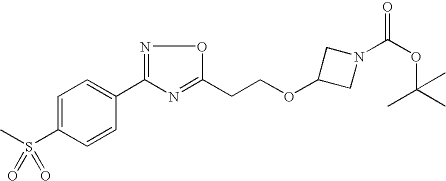 | 3-{2-[3-(4-Methanesulfonyl phenyl)-[1,2,4]oxadiazol-5- yl]ethoxy}azetidine-1-carboxylic acid tert-butyl ester | 3.45 | 441.1 [M + H + NH₃]⁺ |
| 113 | 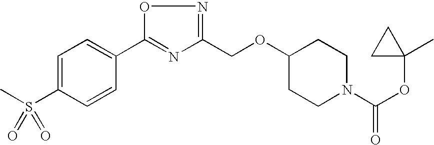 | 4-[5-(4-Methanesulfonylphenyl)- [1,2,4]oxadiazol-3-ylmethoxy] piperidine-1-carboxylic acid 1- methylcyclopropyl ester | 3.42 | 436.1 [M + H]⁺ |
| 114 | 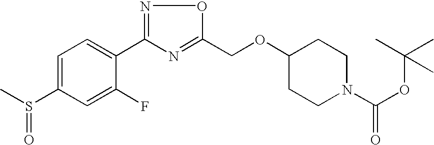 | 4-[3-(2-Fluoro-4-methane sulfinylphenyl)-[1,2,4] oxadiazol-5-ylmethoxy] piperidine-1-carboxylic acid tert- butyl ester | 3.37 | 440.1 [M + H]⁺ |
| 115 | 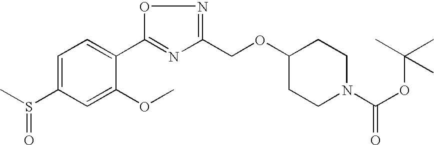 | 4-[5-(4-Methanesulfinyl-2- methoxyphenyl)-[1,2,4] oxadiazol-3-ylmethoxy] piperidine-1-carboxylic acid tert- butyl ester | 3.34 | 452.2 [M + H]⁺ |
| 116 | 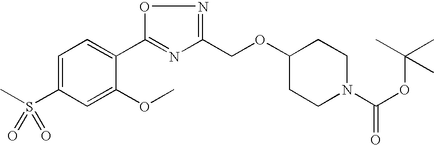 | 4-[5-(4-Methanesulfonyl-2- methoxyphenyl)- [1,2,4]oxadiazol-3-ylmethoxy] piperidine-1-carboxylic acid tert- butyl ester | 3.52 | 468.2 [M + H]⁺ |
| 117 | 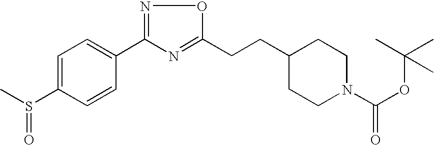 | 4-{2-[3-(4-Methanesulfinyl phenyl)-[1,2,4]oxadiazol-5-yl] ethyl}piperidine-1-carboxylic acid tert-butyl ester | 3.62 | 420.2 [M + H]⁺ |
| 118 | 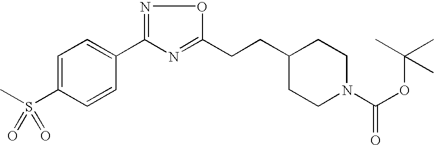 | 4-{2-[3-(4-Methanesulfonyl phenyl)-[1,2,4]oxadiazol-5-yl] ethyl}piperidine-1-carboxylic acid tert-butyl ester | 3.84 | 436.2 [M + H]⁺ |
| 119 | 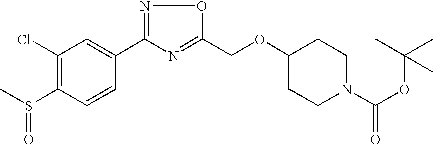 | 4-[3-(3-Chloro-4-methane sulfinylphenyl)-[1,2,4]oxadiazol- 5-ylmethoxy]piperidine-1- carboxylic acid tert-butyl ester | 3.65 | 456.1 [M + H]⁺ |
| 120 | 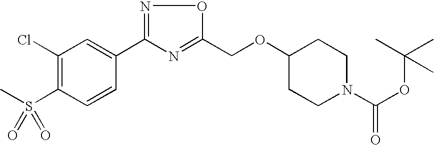 | 4-[3-(3-Chloro-4-methane sulfonylphenyl)-[1,2,4]oxadiazol- 5-ylmethoxy]piperidine-1- carboxylic acid tert-butyl ester | 3.77 | 472.2 [M + H]⁺ |
| 121 | 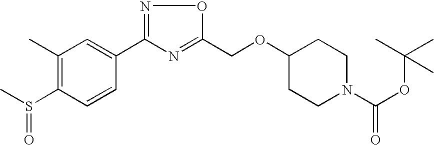 | 4-[3-(4-Methanesulfinyl-3- methylphenyl)-[1,2,4]oxadiazol- 5-ylmethoxy]piperidine-1- carboxylic acid tert-butyl ester | 3.51 | 436.2 [M + H]⁺ |
| 122 | 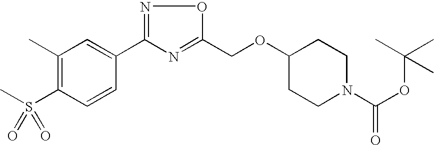 | 4-[3-(4-Methanesulfonyl-3- methylphenyl)-[1,2,4]oxadiazol- 5-ylmethoxy]piperidine-1- carboxylic acid tert-butyl ester | 3.74 | 452.2 [M + H]⁺ |
| 123 | 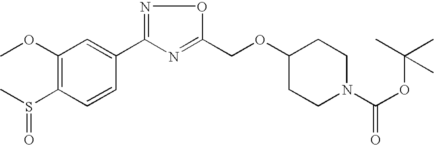 | 4-[3-(4-Methanesulfinyl-3- methoxyphenyl)-[1,2,4] oxadiazol-5-ylmethoxy] piperidine-1-carboxylic acid tert-butyl ester | 3.49 | 452.2 [M + H]⁺ |
| 124 | 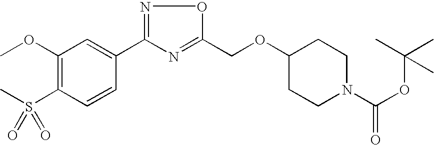 | 4-[3-(4-Methanesulfonyl-3- methoxyphenyl)-[1,2,4] oxadiazol-5-ylmethoxy] piperidine-1-carboxylic acid tert- butyl ester | 3.64 | 468.2 [M + H]⁺ |
| 125 | 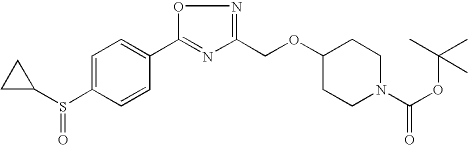 | 4-[5-(4-Cyclopropanesulfinyl phenyl)-[1,2,4]oxadiazol-3-yl methoxy]piperidine-1-carboxylic acid tert-butyl ester | 3.45 | 448.2 [M + H]⁺ |
| 126 | 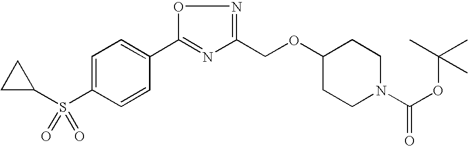 | 4-[5-(4-Cyclopropanesulfonyl phenyl)-[1,2,4]oxadiazol-3-yl methoxy]piperidine-1-carboxylic acid tert-butyl ester | 3.70 | 464.2 [M + H]⁺ |
| 127 | 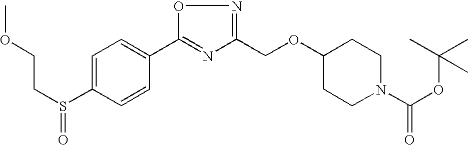 | 4-{5-[4-(2-Methoxyethane sulfinyl)phenyl]-[1,2,4]oxadiazol- 3-ylmethoxy}piperidine-1- carboxylic acid tert-butyl ester | 3.44 | 466.2 [M + H]⁺ |
| 128 | 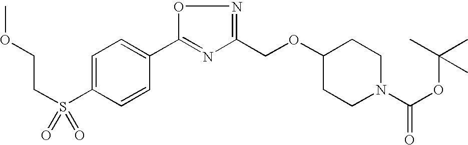 | 4-{5-[4-(2-Methoxyethane sulfonyl)phenyl]-[1,2,4] oxadiazol-3-ylmethoxy} piperidine-1-carboxylic acid tert- butyl ester | 3.61 | 482.2 [M + H]⁺ |
| 129 | 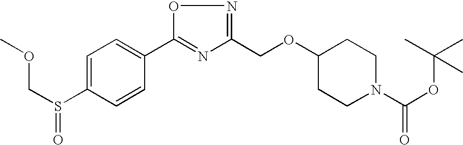 | 4-[5-(4-Methoxymethanesulfinyl phenyl)-[1,2,4]oxadiazol-3-yl methoxy]piperidine-1-carboxylic acid tert-butyl ester | 3.40 | 452.2 [M + H]⁺ |
| 130 | 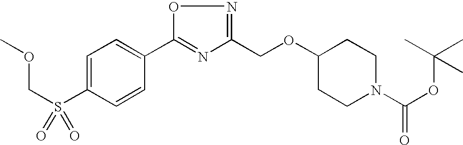 | 4-[5-(4-Methoxymethane sulfonylphenyl)-[1,2,4]oxadiazol- 3-ylmethoxy]piperidine-1- carboxylic acid tert-butyl ester | 3.64 | 468.2 [M + H]⁺ |
| 131 | 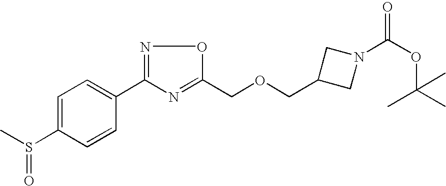 | 3-[3-(4-Methanesulfinylphenyl)- [1,2,4]oxadiazol-5-ylmethoxy methyl]azetidine-1-carboxylic acid tert-butyl ester | 3.27 | 408.2 [M + H]⁺ |
| 132 | 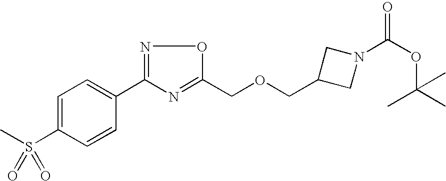 | 3-[3-(4-Methanesulfonylphenyl)- [1,2,4]oxadiazol-5-ylmethoxy methyl]azetidine-1-carboxylic acid tert-butyl ester | 3.45 | 424.2 [M + H]⁺ |
| 133 | 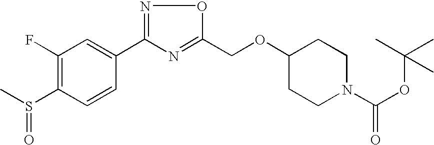 | 4-[3-(3-Fluoro-4-methane sulfinylphenyl)-[1,2,4]oxadiazol- 5-ylmethoxy]piperidine-1- carboxylic acid tert-butyl ester | 3.59 | 440.2 [M + H]⁺ |
| 134 | 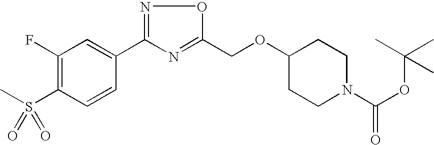 | 4-[3-(3-Fluoro-4-methane sulfonylphenyl)-[1,2,4]oxadiazol- 5-ylmethoxy]piperidine-1- carboxylic acid tert-butyl ester | 3.70 | 456.1 [M + H]⁺ |
| 135 | 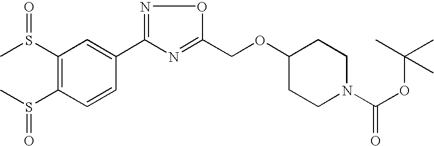 | 4-[3-(3,4-Bismethanesulfinyl phenyl)-[1,2,4]oxadiazol-5-yl methoxy]piperidine-1-carboxylic acid tert-butyl ester | 3.14 | 484.2 [M + H]⁺ |
| 136 | 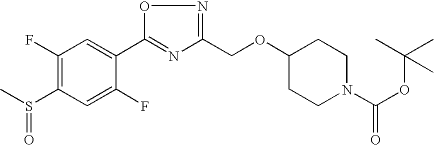 | 4-[5-(2,5-Difluoro-4-methane sulfinylphenyl)-[1,2,4]oxadiazol- 3-ylmethoxy]piperidine-1- carboxylic acid tert-butyl ester | 3.20 | 458.0 [M + H]⁺ |
| 137 | 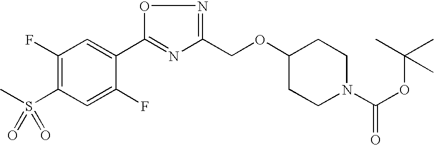 | 4-[5-(2,5-Difluoro-4-methane sulfonylphenyl)-[1,2,4]oxadiazol- 3-ylmethoxy]piperidine-1- carboxylic acid tert-butyl ester | 3.32 | 474.0 [M + H]⁺ |
| 138 | 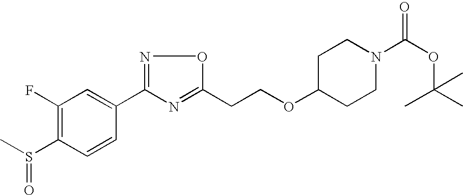 | 4-{2-[3-(3-Fluoro-4-methane sulfinylphenyl)-[1,2,4]oxadiazol- 5-yl]ethoxy}piperidine-1- carboxylic acid tert-butyl ester | 3.56 | 453.1 [M + H]⁺ |
| 139 | 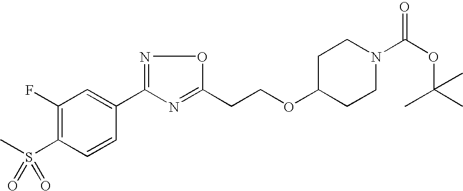 | 4-{2-[3-(3-Fluoro-4-methane sulfonylphenyl)-[1,2,4]oxadiazol- 5-yl]ethoxy}piperidine-1- carboxylic acid tert-butyl ester | 3.77 | 470.1 [M + H]⁺ |
| 140 | 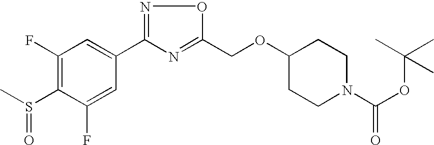 | 4-[3-(3,5-Difluoro-4-methane sulfinylphenyl)-[1,2,4]oxadiazol- 5-ylmethoxy]piperidine-1- carboxylic acid tert-butyl ester | 3.40 | 458.0 [M + H]⁺ |
| 141 | 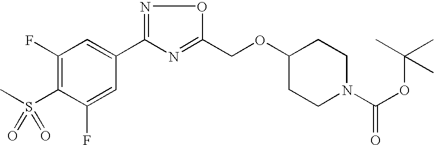 | 4-[3-(3,5-Difluoro-4-methane sulfonylphenyl)-[1,2,4]oxadiazol- 5-ylmethoxy]piperidine-1- carboxylic acid tert-butyl ester | 3.61 | 474.0 [M + H]⁺ |
| 142 | 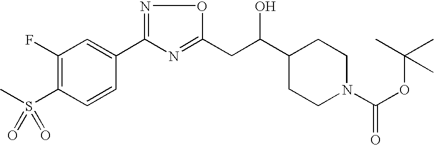 | 4-{2-[3-(3-Fluoro-4-methane sulfonylphenyl)-[1,2,4]oxadiazol- 5-yl]-1-hydroxyethyl}piperidine- 1-carboxylic acid tert-butyl ester | 3.24 | 470.0 [M + H]⁺ |
| 143 | 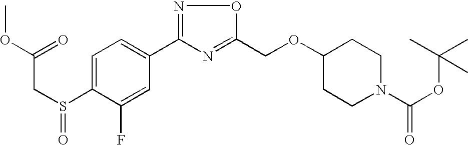 | 4-[3-(3-Fluoro-4-methoxy carbonylmethanesulfinyl phenyl)-[1,2,4]oxadiazol-5-yl methoxy]piperidine-1-carboxylic acid tert-butyl ester | 3.65 | 498.1 [M + H]⁺ |
| 144 | 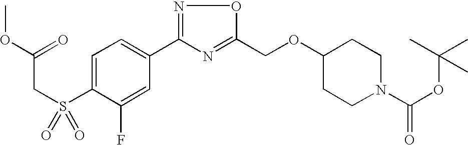 | 4-[3-(3-Fluoro-4-methoxy carbonylmethanesulfonylphenyl)- [1,2,4]oxadiazol-5-ylmethoxy] piperidine-1-carboxylic acid tert- butyl ester | 3.84 | 514.0 [M + H]⁺ |
| 145 | 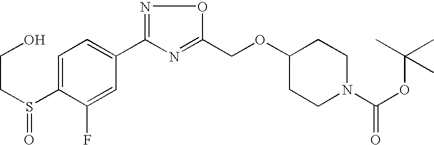 | 4-{3-[3-Fluoro-4-(2- hydroxyethanesulfinyl)phenyl]- [1,2,4]oxadiazol-5-ylmethoxy} piperidine-1-carboxylic acid tert- butyl ester | 3.31 | 470.1 [M + H]⁺ |
| 146 | 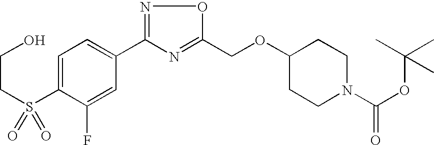 | 4-{3-[3-Fluoro-4-(2-hydroxy ethanesulfonyl)phenyl]-[1,2,4] oxadiazol-5-ylmethoxy} piperidine-1-carboxylic acid tert- butyl ester | 3.47 | 486.0 [M + H]⁺ |

### Example 147

### 4-[5-(4-Methanesulfonylphenyl)-[1,2,4]oxadizol-3-ylmethoxy]piperidine-1-carboxylic acid 1-methylcyclobutyl ester

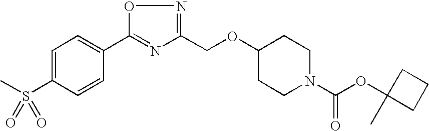

A solution of 4-[5-(4-methanesulfonylphenyl)-[1,2,4]oxadizol-3-ylmethoxy]piperidine-1-carboxylic acid tert-butyl ester (Example 2, 760 mg, 1.74 mmol) in DCM (8 mL) was treated with trifluoroacetic acid (3.8 mL). After 18 h, the solvent was evaporated and the residue taken up in EtOAc (80 mL) and quickly washed with 2 M aqueous NaOH (2×10 mL), ensuring the washings were of pH≧14. The organic phase was dried (MgSO₄) and evaporated to give 4-[5-(4-methanesulfonylphenyl)-[1,2,4]oxadiazol-3-ylmethoxy]piperidine: RT=2.07 min (method 2), m/z (ES⁺)=338 [M+H]⁺.

In a separate vessel, triphosgene (173 mg, 583 μmol) was added to a solution of 1-methylcyclobutanol (38 mg, 440 μmol) in THF (4 mL). After stirring for 1 h, triethylamine (123 μL, 880 μmol) was added and the stirring continued for a further 20 min, whereupon this milky solution of chloroformate was added quickly to a solution of the 4-[5-(4-methanesulfonylphenyl)-[1,2,4]oxadiazol-3-ylmethoxy]piperidine (51 mg, 150 μmol), prepared above, in dry THF (2 mL). After stirring for 17 h, the reaction was diluted with DCM and washed with water. The aqueous layer was re-extracted with DCM and the combined organic extracts dried (MgSO₄). The solvent was removed and the residue purified by RP-HPLC to give the title compound: δ_{H} (CDCl₃) 1.56 (3H, s), 1.66 (3H, m), 1.80 (1H, m), 1.93 (2H, m), 2.13 (2H, m), 2.31 (2H, m), 3.13 (3H, s), 3.15 (2H, m), 3.73 (1H, m), 3.82 (2H, m), 4.77 (2H, s), 8.14 (2H, d), 8.38 (2H, d); RT=3.61 (method 2), m/z (ES⁺)=450.1 [M+H]⁺.

The carbamates listed in Table 5 were synthesised by reacting the appropriate piperidine with the requisite chloroformates, according to the methods outlined in Example 147.

TABLE 5

| Eg | Structure | Name | RT (min) | m/z |
|---|---|---|---|---|
| 148 | 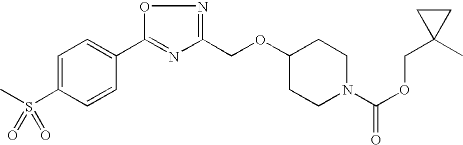 | 4-[5-(4-Methanesu1fonylphenyl)- [1,2,4]oxadiazol-3-ylmethoxy] piperidine-1-carboxylic acid 1- methylcyclopropylmethyl ester | 3.56 | 450.2 [M + H]⁺ |
| 149 | 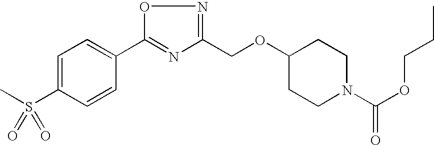 | 4-[5-(4-Methanesulfonylphenyl)- [1,2,4]oxadiazol-3-ylmethoxy] piperidine-1-carboxylic acid propyl ester | 3.47 | 424.1 [M + H]⁺ |
| 150 | 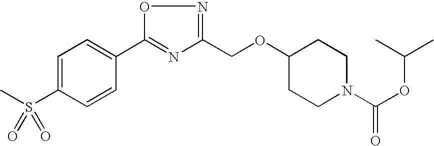 | 4-[5-(4-Methanesulfonylphenyl)- [1,2,4]oxadiazol-3-ylmethoxy] piperidine-1-carboxylic acid isopropyl ester | 3.40 | 424.1 [M + H]⁺ |
| 151 | 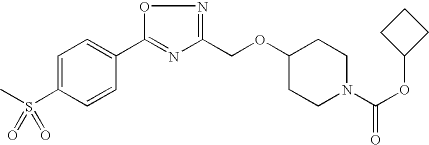 | 4-[5-(4-Methanesulfonylphenyl)- [1,2,4]oxadiazol-3-ylmethoxy] piperidine-1-carboxylic acid cyclobutyl ester | 3.51 | 436.1 [M + H]⁺ |
| 152 | 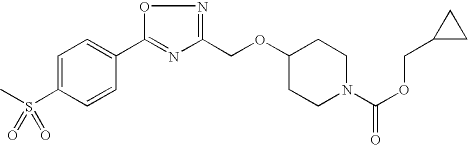 | 4-[5-(4-Methanesulfonylphenyl)- [1,2,4]oxadiazol-3-ylmethoxy] piperidine-1-carboxylic acid cyclopropylmethyl ester | 3.47 | 436.2 [M + H]⁺ |
| 153 | 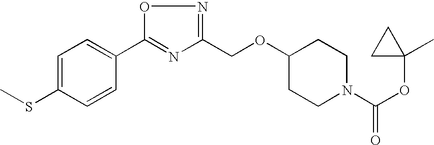 | 4-[5-(4-Methylsulfanylphenyl)- [1,2,4]oxadiazol-3-ylmethoxy] piperidine-1-carboxylic acid 1- methylcyclopropyl ester | 3.99 | 404.1 [M + H]⁺ |
| 154 | 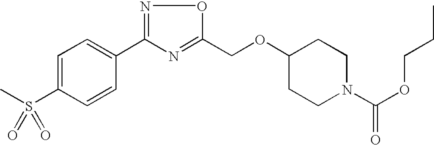 | 4-[3-(4-Methanesulfonylphenyl)- [1,2,4]oxadiazol-5-ylmethoxy] piperidine-1-carboxylic acid propyl ester | 3.45 | 424.1 [M + H]⁺ |
| 155 | 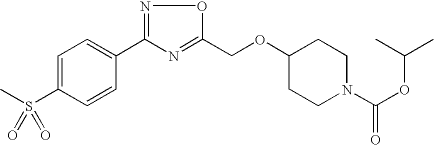 | 4-[3-(4-Methanesulfonylphenyl)- [1,2,4]oxadiazol-5-ylmethoxy] piperidine-1-carboxylic acid isopropyl ester | 3.21 | 424.2 [M + H]⁺ |
| 156 | 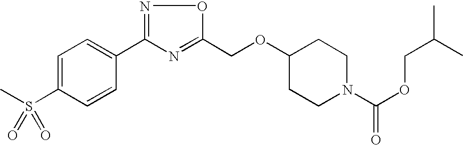 | 4-[3-(4-Methanesulfonylphenyl)- [1,2,4]oxadiazol-5-ylmethoxy] piperidine-1-carboxylic acid isobutyl ester | 3.67 | 438.1 [M + H]⁺ |
| 157 | 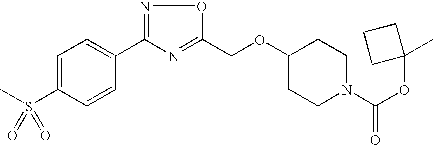 | 4-[3-(4-Methanesulfonylphenyl)- [1,2,4]oxadiazol-5-ylmethoxy] piperidine-1-carboxylic acid 1- methylcyclobutyl ester | 3.65 | 450.2 [M + H]⁺ |
| 158 | 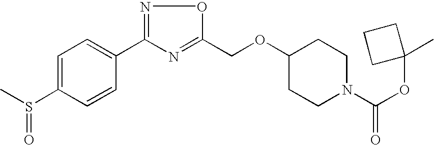 | 4-[3-(4-Methanesulfinylphenyl)- [1,2,4]oxadiazol-5-ylmethoxy]- piperidine-1-carboxylic acid 1- methylcyclobutyl ester | 3.47 | 434.2 [M + H]⁺ |
| 159 | 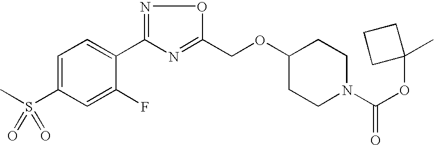 | 4-[3-(2-Fluoro-4-methane sulfonylphenyl)-[1,2,4]oxadiazol- 5-ylmethoxy]piperidine-1- carboxylic acid 1-methylcyclo butyl ester | 3.74 | 468.1 [M + H]⁺ |
| 160 | 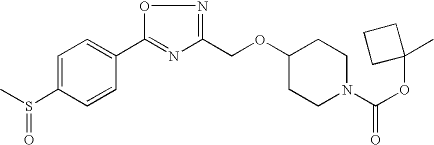 | 4-[5-(4-Methanesulfinylphenyl)- [1,2,4]oxadiazol-3-ylmethoxy] piperidine-1-carboxylic acid 1- methylcyclobutyl ester | 3.36 | 434.1 [M + H]⁺ |
| 161 | 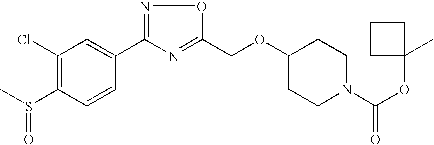 | 4-[3-(3-Chloro-4-methane sulfinylphenyl)-[1,2,4]oxadiazol- 5-ylmethoxy]piperidine-1- carboxylic acid 1-methylcyclo butyl ester | 3.72 | 468.1 [M + H]⁺ |
| 162 | 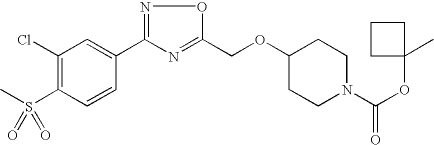 | 4-[3-(3-Chloro-4-methane sulfonylphenyl)-[1,2,4]oxadiazol- 5-ylmethoxy]piperidine-1- carboxylic acid 1-methylcyclo butyl ester | 3.69 | 484.2 [M + H]⁺ |

### Example 163

### 4-{5-[4-(3-Methoxypropylcarbamoyl)phenyl]-[1,2,4]oxadiazol-3-ylmethoxy}piperidine-1-carboxylic acid tert-butyl ester

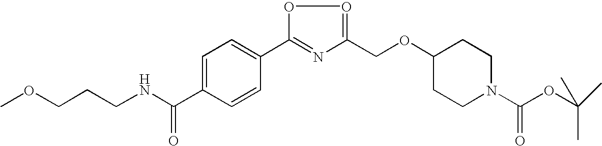

A vigorously-stirred suspension of 4-[5-(4-methoxycarbonylphenyl)-[1,2,4]oxadiazol-3-ylmethoxy]piperidine-1-carboxylic acid tert-butyl ester (Example 35, 450 mg, 1.08 mmol) in MeOH (20 mL) was treated with 2 M aqueous NaOH (1 mL). After 2 h, the methanol was evaporated and water (5 mL) added. This aqueous mixture was washed with EtOAc (2×10 mL), acidified to pH 4 using glacial acetic acid and extracted with EtOAc (3×20 mL). The combined organics were washed with brine (10 mL) and dried (MgSO₄). Evaporation of the solvent afforded 4-[5-(4-carboxyphenyl)-[1,2,4]oxadiazol-3-ylmethoxy]piperidine-1-carboxylic acid tert-butyl ester: RT=3.57 min (method 2), m/z (ES⁺)=404.1 [M+H]⁺.

A stirred solution of this acid (40 mg, 100 μmol) and HOBt (17.5 mg, 130 μmol) in dry THF (2 mL) was treated with EDC (25 mg, 130 μmol). After 20 min, 3-methoxypropylamine (21 μL, 200 μmol) was added, and stirring continued overnight. The mixture was diluted with EtOAc (15 mL), washed with water, saturated aqueous sodium carbonate, brine, dried and evaporated to afford the title compound: δ_{H} (CDCl₃) 1.39 (9H, s), 1.56 (2H, m), 1.85 (4H, m), 3.04 (2H, m), 3.34 (3H, s), 3.54 (4H, m), 3.63 (1H, m), 3.82 (2H, m), 4.68 (2H, s), 7.02 (1H, br t), 7.85 (2H, d), 8.16 (2H, d); RT=3.54 min (method 2), m/z (ES⁺)=475.2 [M+H]⁺.

The compounds in Table 6 were synthesized in a manner similar to that described in Example 163.

TABLE 6

| | | | RT | m/z |
|---|---|---|---|---|
| Eg | Structure | Name | (min) | (ES⁺) |
| 164 | 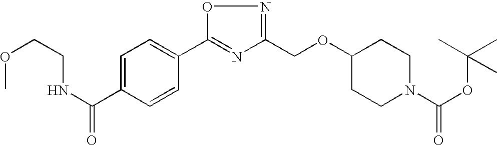 | 4-{5-[4-(2-Methoxyethyl carbamoyl)phenyl]-[1,2,4] oxadiazol-3-ylmethoxy} piperidine-1-carboxylic acid tert-butyl ester | 3.42 | 461.2 [M + H]⁺ |
| 165 | 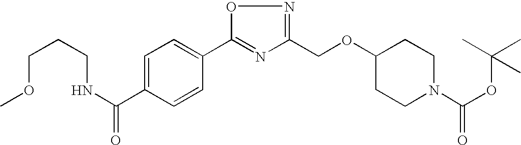 | 4-{5-[4-(3-Methoxypropyl carbamoyl)phenyl]-[1,2,4] oxadiazol-3-ylmethoxy} piperidine-1-carboxylic acid tert-butyl ester | 3.54 | 475.2 [M + H]⁺ |
| 166 | 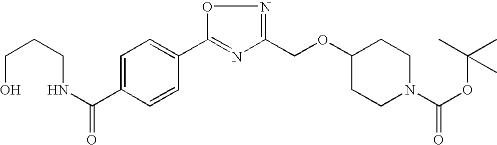 | 4-{5-[4-(3-Hydroxypropyl carbamoyl)phenyl]-[1,2,4] oxadiazol-3-ylmethoxy} piperidine-1-carboxylic acid tert-butyl ester | 3.19 | 461.3 [M + H]⁺ |
| 167 | 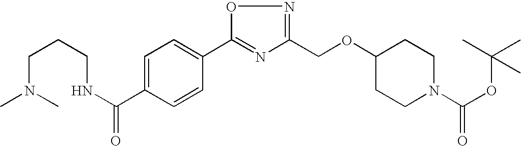 | 4-{5-[4-(3-Dimethylamino- propylcarbamoyl)phenyl]- [1,2,4]oxadiazol-3- ylmethoxy}piperidine-1- carboxylic acid tert-butyl ester | 2.72 | 488.3 [M + H]⁺ |
| 168 | 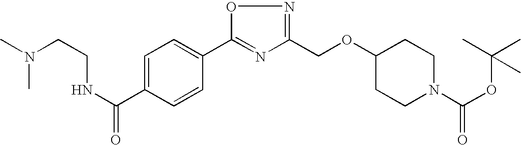 | 4-{5-[4-(2-Dimethylamino- ethylcarbamoyl)phenyl]- [1,2,4]oxadiazol-3- ylmethoxy}piperidine-1- carboxylic acid tert-butyl ester | 2.61 | 474.3 [M + H]⁺ |
| 169 | 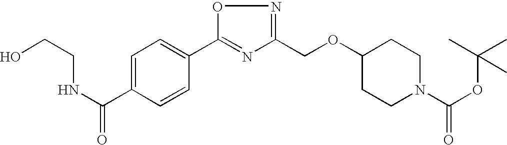 | 4-{5-[4-(2-Hydroxyethyl carbamoyl)phenyl]-[1,2,4] oxadiazol-3-ylmethoxy} piperidine-1-carboxylic acid tert-butyl ester | 3.17 | 447.2 [M + H]⁺ |
| 170 | 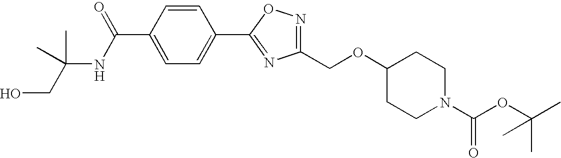 | 4-{5-[4-(2-Hydroxy-1,1- dimethylethylcarbamoyl) phenyl]-[1,2,4]oxadiazol-3- ylmethoxy}piperidine-1- carboxylic acid tert-butyl ester | 5.09 | 475.1 [M + H]⁺ |
| 171 | 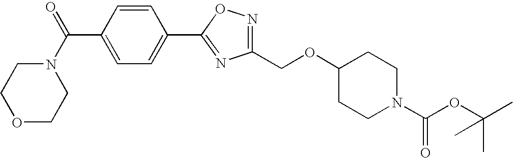 | 4-{5-[4-(Morpholine-4- carbonyl)phenyl]-[1,2,4] oxadiazol-3-ylmethoxy} piperidine-1-carboxylic acid tert-butyl ester | 5.02 | 473.1 [M + H]⁺ |
| 172 | 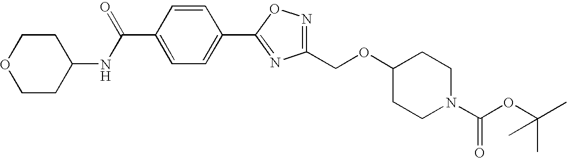 | 4-{5-[4-(Tetrahydropyran- 4-ylcarbamoyl)phenyl]- [1,2,4]oxadiazol-3- ylmethoxy}piperidine-1- carboxylic acid tert-butyl ester | 5.07 | 487.1 [M + H]⁺ |
| 173 | 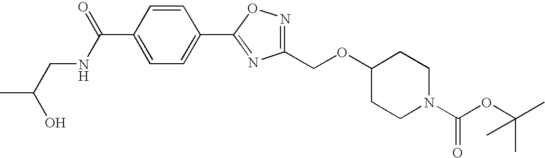 | 4-{5-[4-(2-Hydroxypropyl carbamoyl)phenyl]-[1,2,4] oxadiazol-3-ylmethoxy} piperidine-1-carboxylic acid tert-butyl ester | 4.67 | 461.1 [M + H]⁺ |
| 174 | 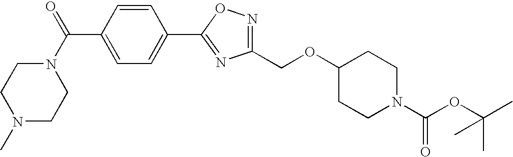 | 4-{5-[4-(4-Methyl- piperazine-1-carbonyl) phenyl]-[1,2,4]oxadiazol-3- ylmethoxy}piperidine-1- carboxylic acid tert- butyl ester | 4.89 | 486.1 [M + H]⁺ |
| 175 | 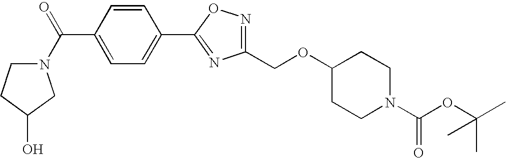 | 4-{5-[4-(3-Hydroxy- pyrrolidine-1-carbonyl) phenyl]-[1,2,4]oxadiazol-3- ylmethoxy}piperidine-1- carboxylic acid tert- butyl ester | 4.53 | 473.1 [M + H]⁺ |
| 176 | 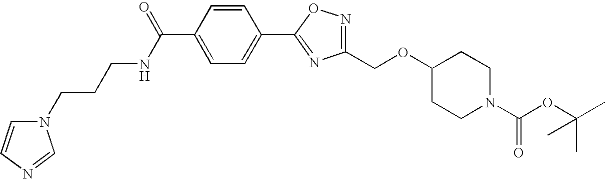 | 4-{5-[4-(3-Imidazol-1- ylpropylcarbamoyl)phenyl]- [1,2,4]oxadiazol-3- ylmethoxy}piperidine-1- carboxylic acid tert- butyl ester | 4.87 | 511.1 [M + H]⁺ |
| 177 | 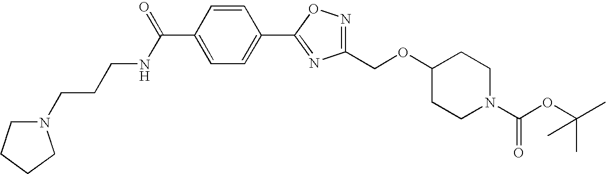 | 4-{5-[4-(3-Pyrrolidin-1-yl propylcarbamoyl)phenyl]- [1,2,4]oxadiazol-3-yl- methoxy}piperidine-1- carboxylic acid tert- butyl ester | 7.52 | 514.2 [M + H]⁺ |
| 178 | 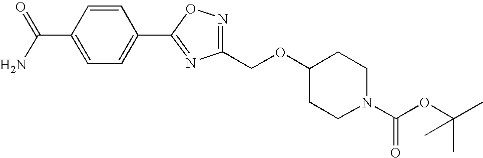 | 4-[5-(4-Carbamoylphenyl)- [1,2,4]oxadiazol-3-yl- methoxy]piperidine-1- carboxylic acid tert- butyl ester | 4.70 | 403.1 [M + H]⁺ |
| 179 | 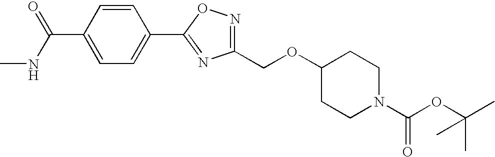 | 4-[5-(4-Methylcarbamoyl phenyl)-[1,2,4]oxadiazol-3- ylmethoxy]piperidine-1- carboxylic acid tert-butyl ester | 4.89 | 417.0 [M + H]⁺ |
| 180 | 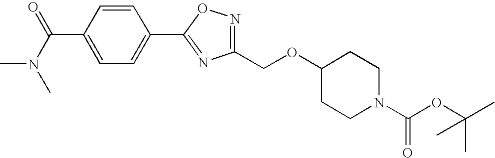 | 4-[5-(4-Dimethylcarbamoyl phenyl)-[1,2,4]oxadiazol-3- ylmethoxy]piperidine-1- carboxylic acid tert-butyl ester | 5.07 | 431.1 [M + H]⁺ |
| 181 | 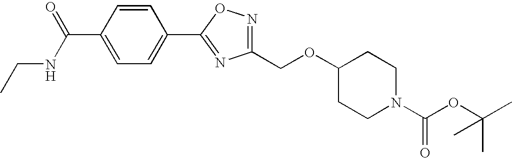 | 4-[5-(4-Ethylcarbamoyl- phenyl)-[1,2,4]oxadiazol-3- ylmethoxy]piperidine-1- carboxylic acid tert- butyl ester | 5.18 | 431.1 [M + H]⁺ |
| 182 | 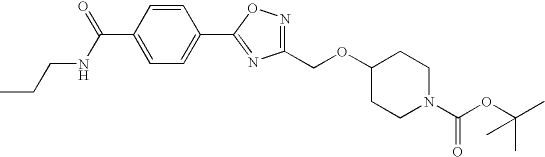 | 4-[5-(4-Propylcarbamoyl phenyl)-[1,2,4]oxadiazol-3- ylmethoxy]piperidine-1- carboxylic acid tert-butyl ester | 4.53 | 445.1 [M + H]⁺ |
| 183 | 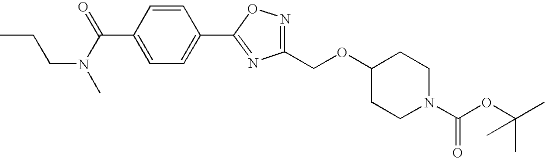 | 4-{5-[4-(Methylpropyl carbamoyl)phenyl]-[1,2,4] oxadiazol-3-ylmethoxy} piperidine-1-carboxylic acid tert-butyl ester | 4.75 | 459.1 [M + H]⁺ |
| 184 | 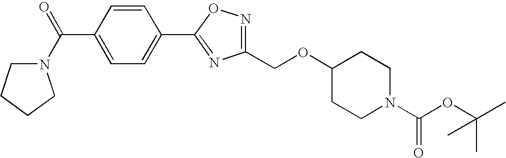 | 4-{5-[4-(Pyrrolidine-1- carbonyl)phenyl]-[1,2,4] oxadiazol-3-ylmethoxy} piperidine-1-carboxylic acid tert-butyl ester | 5.39 | 457.1 [M + H]⁺ |
| 185 | 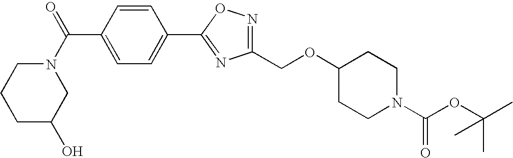 | 4-{5-[4-(3-Hydroxy- piperidine-1-carbonyl) phenyl]-[1,2,4]oxadiazol-3- ylmethoxy}piperidine-1- carboxylic acid tert-butyl ester | 4.79 | 487.1 [M + H]⁺ |
| 186 | 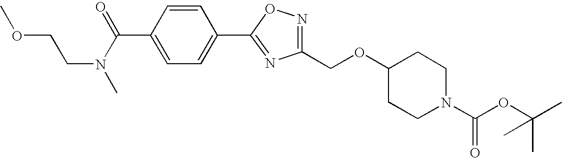 | 4-(5-{4-[(2-Methoxyethyl) methylcarbamoyl]phenyl}- [1,2,4]oxadiazol-3- ylmethoxy)piperidine-1- carboxylic acid tert- butyl ester | 5.20 | 475.1 [M + H]⁺ |
| 187 | 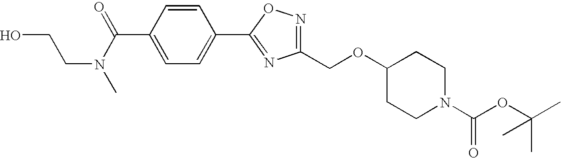 | 4-(5-{4-[(2-Hydroxyethyl) methylcarbamoyl]phenyl}- [1,2,4]oxadiazol-3- ylmethoxy)piperidine-1- carboxylic acid tert- butyl ester | 4.57 | 461.1 [M + H]⁺ |
| 188 | 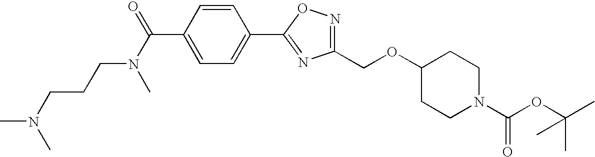 | 4-(5-{4-[(3-Dimethylamino propyl)methylcarbamoyl] phenyl}-[1,2,4]oxadiazol-3- ylmethoxy)piperidine-1- carboxylic acid tert-butyl ester | 5.93 | 502.2 [M + H]⁺ |
| 189 | 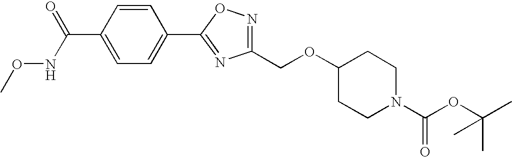 | 4-[5-(4-Methoxycarbamoyl phenyl)-[1,2,4]oxadiazol-3- ylmethoxy]piperidine-1- carboxylic acid tert-butyl ester | 3.10 | 433.1 [M + H]⁺ |
| 190 | 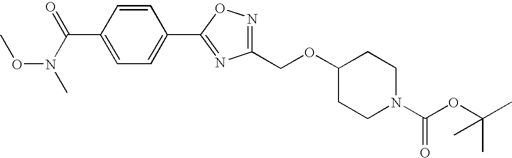 | 4-{5-[4-(Methoxymethyl carbamoyl)phenyl]- [1,2,4]oxadiazol-3- ylmethoxy}piperidine-1- carboxylic acid tert- butyl ester | 5.40 | 447.1 [M + H]⁺ |
| 191 | 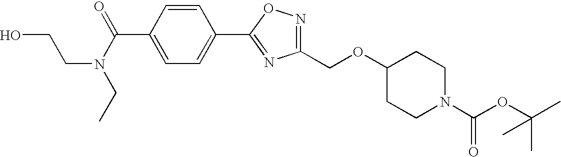 | 4-(5-{4-[Ethyl-(2-hydroxy- ethyl)carbamoyl]phenyl}- [1,2,4]oxadiazol-3-yl- methoxy)piperidine-1- carboxylic acid tert- butyl ester | 4.84 | 475.2 [M + H]⁺ |
| 192 | 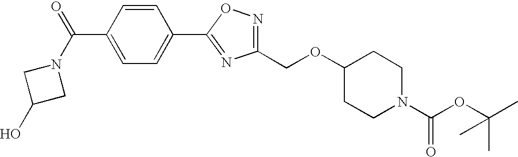 | 4-{5-[4-(3-Hydroxy- azetidine-1-carbonyl) phenyl]-[1,2,4]oxadiazol-3- ylmethoxy}piperidine-1- carboxylic acid tert- butyl ester | 4.57 | 459.1 [M + H]⁺ |
| 193 | 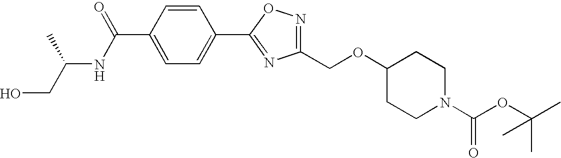 | 4-{5-[4-((S)-2-Hydroxy-1- methylethylcarbamoyl) phenyl]-[1,2,4]oxadiazol-3- ylmethoxy}piperidine-1- carboxylic acid tert- butyl ester | 4.68 | 461.1 [M + H]⁺ |
| 194 | 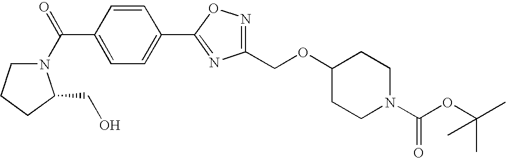 | 4-{5-[4-((S)-2-Hydroxy- methylpyrrolidine-1- carbonyl)phenyl]-[1,2,4] oxadiazol-3-ylmethoxy} piperidine-1-carboxylic acid tert-butyl ester | 4.92 | 487.1 [M + H]⁺ |
| 195 | 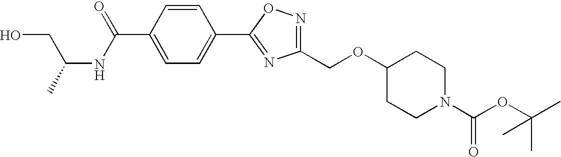 | 4-{5-[4-((R)-2-Hydroxy-1- methylethylcarbamoyl) phenyl]-[1,2,4] oxadiazol-3-ylmethoxy} piperidine-1-carboxylic acid tert-butyl ester | 4.72 | 461.1 [M + H]⁺ |
| 196 | 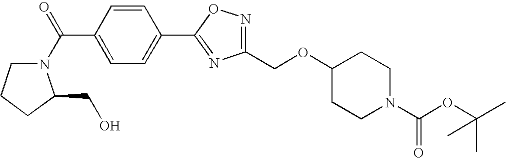 | 4-{5-[4-((R)-2-Hydroxy- methyl pyrrolidine-1- carbonyl)phenyl]-[1,2,4] oxadiazol-3-ylmethoxy} piperidine-1-carboxylic acid tert-butyl ester | 4.93 | 487.1 [M + H]⁺ |
| 197 | 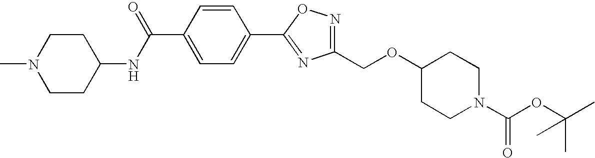 | 4-{5-[4-(1-Methylpiperidin- 4-ylcarbanxoyl)phenyl]- [1,2,4]oxadiazol-3- ylmethoxy}piperidine-1- carboxylic acid tert- butyl ester | 5.62 | 500.1 [M + H]⁺ |
| 198 | 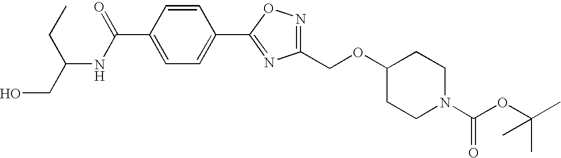 | 4-{5-[4-(1-Hydroxymethyl- propylcarbamoyl)phenyl]- [1,2,4]oxadiazol-3- ylmethoxy}-piperidine-1- carboxylic acid tert- butyl ester | 4.93 | 475.1 [M + H]⁺ |
| 199 | 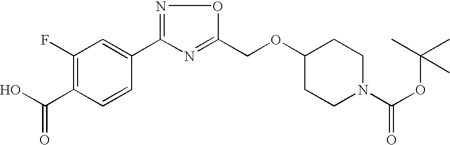 | 4-[3-(4-Carboxy-3-fluoro- phenyl)-[1,2,4]oxadiazol-5- ylmethoxy]piperidine-1- carboxylic acid tert- butyl ester | 3.49 | 422.1 [M + H]⁺ |
| 200 | 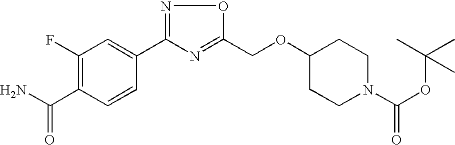 | 4-[3-(4-Carbamoyl-3-fluoro phenyl)-[1,2,4]oxadiazol-5- ylmethoxy]piperidine-1- carboxylic acid tert-butyl ester | 3.40 | 421.1 [M + H]⁺ |
| 201 | 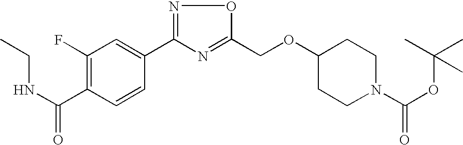 | 4-[3-(4-Ethylcarbamoyl-3- fluorophenyl)-[1,2,4] oxadiazol-5-ylmethoxy] piperidine-1-carboxylic acid tert-butyl ester | 3.65 | 449.1 [M + H]⁺ |
| 202 | 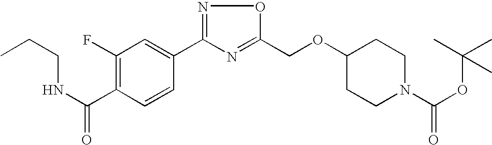 | 4-[3-(3-Fluoro-4-propyl carbamoylphenyl)-[1,2,4] oxadiazol-5-ylmethoxy] piperidine-1-carboxylic acid tert-butyl ester | 3.70 | 463.1 [M + H]⁺ |
| 203 | 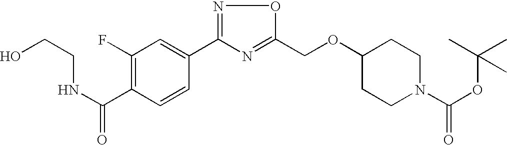 | 4-{3-[3-Fluoro-4-(2- hydroxyethylcarbamoyl) phenyl]-[1,2,4] oxadiazol-5-ylmethoxy} piperidine-1-carboxylic acid tert-butyl ester | 3.24 | 465.1 [M + H]⁺ |
| 204 | 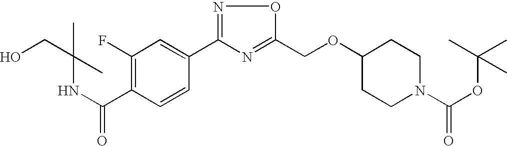 | 4-{3-[3-Fluoro-4-(2- hydroxy-1,1-dimethylethyl- carbamoyl)phenyl]-[1,2,4] oxadiazol-5-ylmethoxy} piperidine-1-carboxylic acid tert-butyl ester | 3.54 | 493.1 [M + H]⁺ |
| 205 | 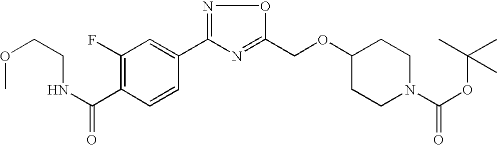 | 4-{3-[3-Fluoro-4-(2- methoxyethylcarbamoyl) phenyl]-[1,2,4]oxadiazol-5- ylmethoxy}piperidine-1- carboxylic acid tert- butyl ester | 3.57 | 479.1 [M + H]⁺ |
| 206 | 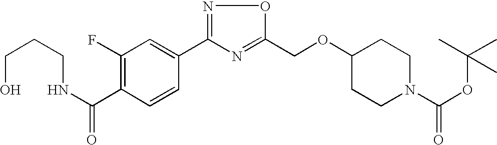 | 4-{3-[3-Fluoro-4-(3- hydroxypropylcarbamoyl) phenyl]-[1,2,4]oxadiazol- 5-ylmethoxy}piperidine-1- carboxylic acid tert- butyl ester | 3.32 | 479.1 [M + H]⁺ |
| 207 | 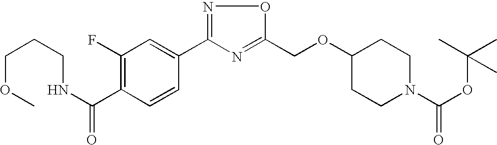 | 4-{3-[3-Fluoro-4-(3- methoxypropylcarbamoyl) phenyl]-[1,2,4] oxadiazol-5-ylmethoxy} piperidine-1-carboxylic acid tert-butyl ester | 3.57 | 493.1 [M + H]⁺ |
| 208 | 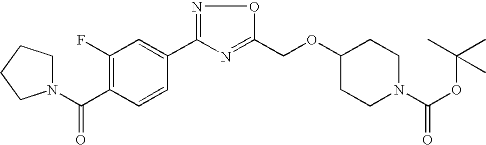 | 4-{3-[3-Fluoro-4- (pyrrolidine-1-carbonyl) phenyl]-[1,2,4] oxadiazol-5-ylmethoxy} piperidine-1-carboxylic acid tert-butyl ester | 3.70 | 475.1 [M + H]⁺ |
| 209 | 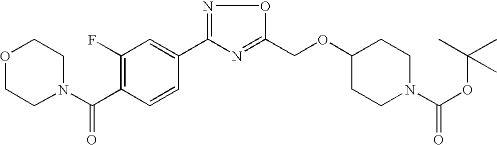 | 4-{3-[3-Fluoro-4- (morpholine-4-carbonyl) phenyl]-[1,2,4] oxadiazol-5-ylmethoxy} piperidine-1-carboxylic acid tert-butyl ester | 3.57 | 491.1 [M + H]⁺ |
| 210 | 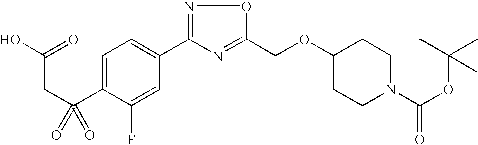 | 4-[3-(4-Carboxymethane sulfonyl-3-fluorophenyl)- [1,2,4]oxadiazol-5-yl- methoxy]piperidine-1- carboxylic acid tert- butyl ester | 3.56 | 500.0 [M + H]⁺ |

### Example 211

### 2-{4-[5-(4-Methanesulfonylphenyl)-[1,2,4]oxadiazol-3-ylmethoxy]piperidine-1-yl}pyrimidine

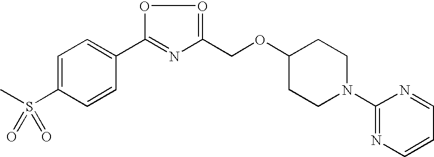

A solution comprised of 4-[5-(4-methanesulfonylphenyl)-[1,2,4]oxadizol-3-ylmethoxy]piperidine (see Example 147, 50 mg, 148 μmol), 2-bromopyrimidine (26 mg, 164 μmol) and DBU (44 μL, 295 μmol) in 1,4-dioxane was stirred until starting material had been consumed. The mixture was concentrated, and purified by column chromatography (EtOAc) to afford the title compound. RT=3.11 min (method 2), m/z (ES⁺)=416.0 [M+H]⁺.

The compounds in Table 7 were synthesised by reaction of 2-bromopyrimidine or 2-fluoropyridine with the appropriate piperidine (synthesised from the corresponding piperidine-1-carboxylic acid tert-butyl ester using the method described in Example 211).

TABLE 7

| Eg | Structure | Name | RT (min) | m/z |
|---|---|---|---|---|
| 212 | 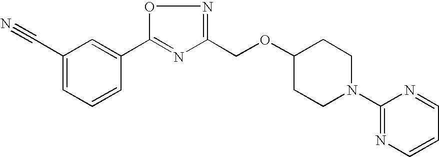 | 3-[3-(1-Pyrimidin-2-ylpiperidin- 4-yloxymethyl)-[1,2,4] oxadiazol-5-yl]benzonitrile | 3.36 | 363.1 [M + H]⁺ |
| 213 | 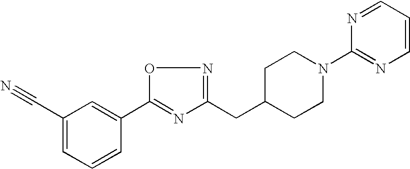 | 3-[3-(1-Pyrimidin-2-ylpiperidin- 4-ylmethyl)-[1,2,4]oxadiazol-5- yl]benzonitrile | 3.47 | 347.1 [M + H]⁺ |
| 214 | 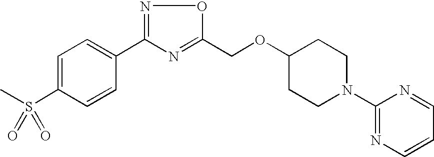 | 2-{4-[3-(4-Methanesulfonyl phenyl)-[1,2,4]oxadiazol-5-yl methoxy]piperidin-1-yl} pyrimidine | 3.17 | 416.1 [M + H]⁺ |
| 215 | 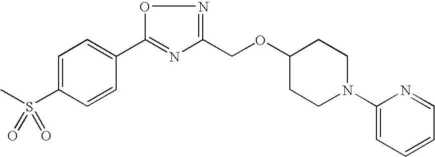 | 4-[5-(4-Methanesulfonylphenyl)- [1,2,4]oxadiazol-3-ylmethoxy]- 3,4,5,6-tetrahydro-2H-[1,2′] bipyridinyl | 2.32 | 415.1 [M + H]⁺ |
| 216 | 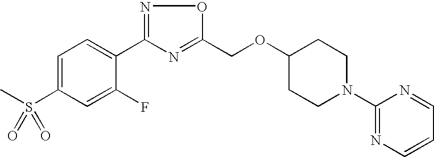 | 2-{4-[3-(2-Fluoro-4-methane sulfonylphenyl)-[1,2,4] oxadiazol-5-ylmethoxy] piperidin-1-yl}pyrimidine | 3.20 | 434.1 [M + H]⁺ |
| 217 | 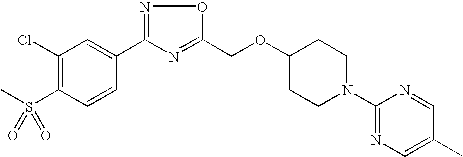 | 2-{4-[3-(3-Chloro-4-methane sulfinylphenyl)-[1,2,4]oxadiazol- 5-ylmethoxy]piperidin-1-yl}-5- methylpyrimidine | 3.36 | 448.1 [M + H]⁺ |
| 218 | 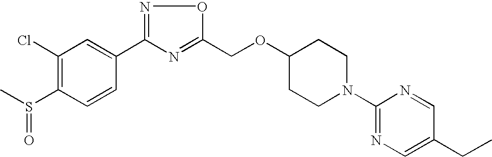 | 2-{4-[3-(3-Chloro-4-methane sulfinylphenyl)-[1,2,4]oxadiazol- 5-ylmethoxy]piperidin-1-yl}-5- ethylpyrimidine | 3.52 | 462.1 [M + H]⁺ |

### Example 219

### 4-{3-[3-Fluoro-4-(2-methoxyethanesulfonyl)phenyl]-[1,2,4]oxadiazol-5-ylmethoxy}piperidine-1-carboxylic acid tert-butyl ester

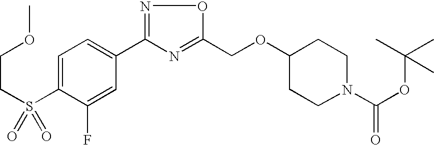

Sodium hydride (11.5 mg of a 60% dispersion in oil, 290 μmol) was suspended in anhydrous DMF (0.5 mL) under argon and cooled to 0° C. A solution of 4-{3-[3-fluoro-4-(2-hydroxyethylsulfanyl)phenyl]-[1,2,4]oxadiazol-5-ylmethoxy}piperidine-1-carboxylic acid tert-butyl ester (Example 69, 104 mg, 229 μmol) in anhydrous DMF (1 mL) was added via cannula and the mixture stirred for 30 min at rt Neat methyl iodide (18 μL, 290 μmol) was added and stirring continued for 18 h. The solvent was removed and ether (10 mL) and water (2 mL) added. The organic was separated, evaporated and the residue purified by preparative tlc (IH-EtOAc 1:1) to afford 4-{3-[3-fluoro-4-(2-methoxyethylsulfanyl)phenyl]-[1,2,4]oxadiazol-5-ylmethoxy}piperidine-1-carboxylic acid tert-butyl ester. RT=4.27 min (method 2); m/z (ES*)=468.1 [M+H]⁺. A sample of this thioether was oxidised using the procedure described for Example 96 and 97 to afford the title sulfone: RT=3.67 min (method 2); m/z (ES⁺)=500.0 [M+H]⁺.

### Example 220

### 4-{2-[3-(4-Methanesulfonylphenyl)-[1,2,4]oxadiazol-5-yloxy]ethyl}piperidine 1-carboxylic acid tert-butyl ester

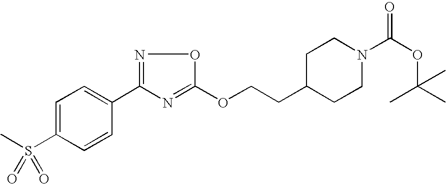

A solution of 4-(2-hydroxyethyl)piperidine-1-carboxylic acid tert-butyl ester (98 mg, 427 μmol) in anhydrous DMF (1.5 mL) was treated with sodium hydride (17 mg of a 60% dispersion in oil, 425 μmol) and the mixture stirred under argon for 35 min. 5-Chloro-3-(4-methanesulfonylphenyl)-[1,2,4]oxadiazole (Preparation 47, 100 mg, 388 μmol) was added in one portion and stirring continued for 20 h. The solvent was removed and the residue taken up in EtOAc (25 mL) and water (10 mL). The organic phase was separated and washed with brine and dried (MgSO₄). The solvent was removed and the residual material purified by column chromatography (IH-EtOAc 1:1) to give the title ether: RT=3.94 min (method 2); m/z (ES⁺)=396.0 [M+H—C₄H₈]⁺.

The 5-alkoxy-[1,2,4]oxadiazoles listed in Table 8 were prepared using a similar method to that described in Example 220.

### Example 221

### 4-(2-{[3-(4-Methanesulfonylphenyl)-[1,2,4]oxadiazol-5-yl]methylamino}ethyl)piperidine-1-carboxylic acid tert-butyl ester

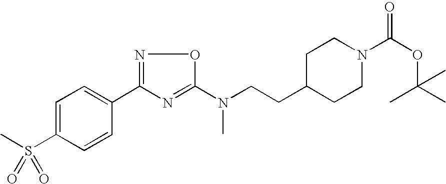

A solution of 4-(2-oxoethyl)piperidine-1-carboxylic acid tert-butyl ester (300 mg, 1.32 mmol) in toluene (1 mL) and methylamine (0.66 mL of a 2 M solution in toluene) was stirred at rt for 1 h and the solvent removed. The residue was dissolved in 1:1 THF/MeOH (2 mL) and sodiumborohydride (60 mg, 1.58 mmol) added. After stirring overnight, the mixture was diluted with EtOAc (50 mL) and washed with water (10 mL), brine (10 mL) and dried (MgSO₄). Evaporation of the solvent afforded crude 4-(2-methylaminoethyl)piperidine-1-carboxylic acid tert-butyl ester which was dissolved in anhydrous DMF (1 mL) and added to 5-chloro-3-(4-methanesulfonylphenyl)-[1,2,4]oxadiazole (Preparation 47, 100 mg, 388 μmol), followed by triethylamine (54 μL, 388 μmol). The mixture was heated at 120° C. for 2 h, cooled and the solvent removed. The residue was purified by RP-HPLC (CH₃CN—H₂O) to afford the title compound. RT=3.95 min (method 2); m/z (ES⁺)=465.1 [M+H]⁺.

The 5-alkylamino[1,2,4]oxadiazoles listed in Table 8 were similarly prepared by reaction of the appropriate amine with 5-chloro-3-(4-methanesulfonylphenyl)-[1,2,4]oxadiazole.

TABLE 8

| Eg | Structure | Name | RT (min) | m/z |
|---|---|---|---|---|
| 222 | 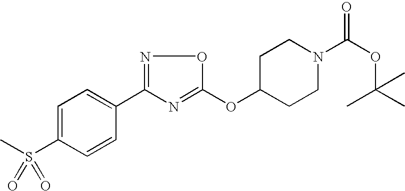 | 4-[3-(4-Methanesulfonylphenyl)- [1,2,4]oxadiazol-5-yloxy] piperidine-1-carboxylic acid tert- butyl ester | 3.74 | 424.0 [M + H]⁺ |
| 223 | 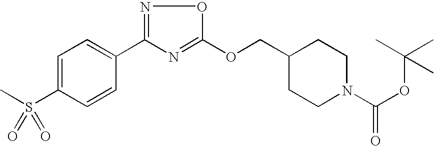 | 4-[3-(4-Methanesulfonylphenyl)- [1,2,4]oxadiazol-5-yloxymethyl] piperidine-1-carboxylic acid tert- butyl ester | 3.79 | 382.0 [M + H − C₄H₈]⁺ |
| 224 | 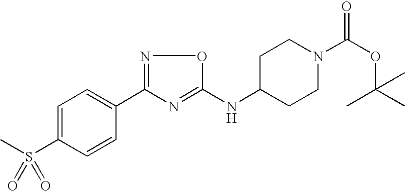 | 4-[3-(4-Methanesulfonylphenyl)- [1,2,4]oxadiazol-5-ylamino] piperidine-1-carboxylic acid tert- butyl ester | 3.52 | 423.1 [M + H]⁺ |
| 225 | 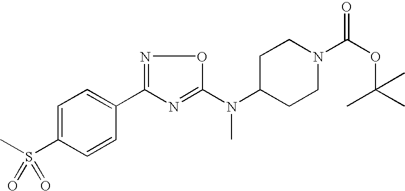 | 4-{[3-(4-Methanesulfonyl phenyl)-[1,2,4]oxadiazol-5-yl] methylamino)piperidine-1- carboxylic acid tert-butyl ester | 3.70 | 437.1 [M + H]⁺ |
| 226 | 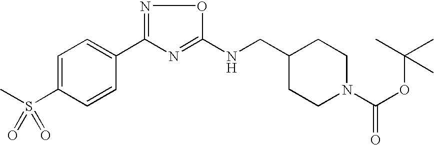 | 4-{[3-(4-Methanesulfonyl phenyl)-[1,2,4]oxadiazol-5- ylamino]methyl}piperidine-1- carboxylic acid tert-butyl ester | 3.49 | 437.1 [M + H]⁺ |
| 227 | 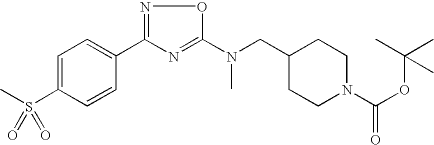 | 4-({[3-(4-Methanesulfonyl phenyl)-[1,2,4]oxadiazol-5- yl]methylamino}methyl) piperidine-1-carboxylic acid tert- butyl ester | 3.60 | 451.1 [M + H]⁺ |

### Example 228

### 4-{2-[3-(3-Fluoromethanesulfonylphenyl)-[1,2,4]oxadiazol-5-yl]acetyl}piperidine-1-carboxylic acid tert-butyl ester

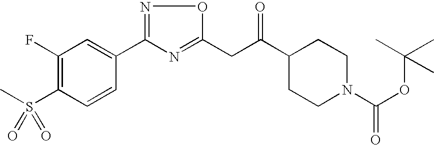

A stirred solution of 4-{2-[3-(3-fluoro-4-methanesulfonylphenyl)-[1,2,4]oxadiazol-5-yl]-1-hydroxyethyl}piperidine-1-carboxylic acid tert-butyl ester (Example 142,230 mg, 490 μmol) in DCM (14 mL) was treated with Dess-Martin periodinane (229 mg, 539 μmol). After 1 h the mixture was poured into 2 M aqueous NaOH (10 mL) and extracted with ether (100 mL). The organic phase was washed with brine, dried (MgSO₄) and evaporated. The residue was purified by column chromatography (IH-EtOAc 2:3) to afford the title compound. RT=3.67 min (method 2); m/z (ES⁺)=468.0 [M+H]⁺.

### Example 229 and 230

### 4-[3-(3-Fluoro-4-methylsulfamoylphenyl)-[1,2,4]oxadiazol-5-ylmethoxy]piperidine-1-carboxylic acid tert-butyl ester and 4-[3-(4-dimethylsulfamoyl-3-fluorophenyl)-[1,2,4]oxadiazol-5-ylmethoxy]piperidine-1-carboxylic acid tert-butyl ester

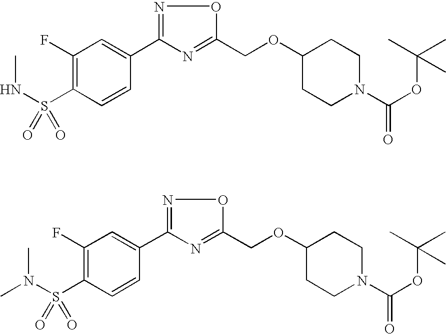

A stirred solution of 4-[3-(3-fluoro-4-sulfamoylphenyl)-[1,2,4]oxadiazol-5-ylmethoxy]piperidine-t-carboxylic acid tert-butyl ester (Example 70, 58 mg, 130 Amos) in anhydrous DMF (3 mL) was treated with sodium hydride (5.6 mg of a 60% dispersion in oil, 140 μmol). After 30 min, neat methyl iodide (8 mL, 130 μmol) was added and stirring was continued for 3 h. The mixture was diluted with EtOAc (50 mL) and washed with water (10 mL) and brine (10 mL) and dried (MgSO₄). The solvent was evaporated and the residue purified by preparative HPLC to afford the title methylsulfonamide: RT=3.57 min (method 2); m/z (ES⁺)=415.0 [M+H−C₄H₈]⁺ and the title dimethylsulfonamide: RT=3.82 min (method 2); m/z (ES⁺)=485.0 [M+H]⁺

### Example 231

### 4-[1-(4-Methanesulfonylphenyl)-1H-[1,2,3]triazol-4-ylmethoxy]piperidine-1-carboxylic acid tert-butyl ester

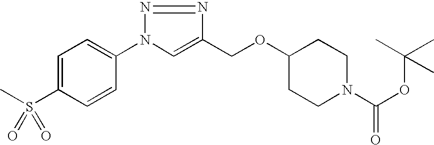

A mixture of 4-bromomethyl-1-(4-methanesulfonylphenyl)-1H-[1,2,3]triazole (Preparation 42, 142 mg, 450 μmol), silver triflate (113 mg, 441 μmol) and 4-hydroxypiperidine-1-carboxylic acid tert-butyl ester (176 mg, 875 μmol) in DCM (5 mL) was stirred for 18 h at rt. After washing with water (5 mL), the solvent was removed and the residue purified by column chromatography (IH-EtOAc 1:1) to give the title compound: RT=3.34 min (method 2); m/z (ES⁺)=437.1 [M+H]⁺.

### Example 232

### 4-[1-(4-Methanesulfonylphenyl)-1H-pyrazol-4-ylmethoxy]piperidine-1-carboxylic acid tert-butyl ester

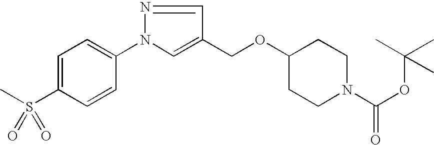

Using the method described in Example 231, 4-bromomethyl-1-(4-methanesulfonylphenyl)-1H-pyrazole (Preparation 43) was converted to the title compound: RT=3.47 min; m/z (ES⁺)=436.1 [M+H]⁺.

### Example 233

### 4-[5-(4-Pentylcyclohexyl)-[1,2,4]oxadiazol-3-yl]phenylamine

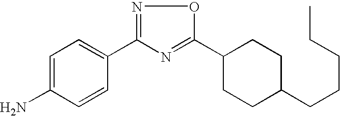

A solution of {4-[5-(4-pentylcyclohexyl)-[1,2,4]oxadiazol-3-yl]phenyl}carbamic acid tert-butyl ester (Example 61, 26 mg, 63 μmol) in a mixture of DCM (1 mL) and trifluoroacetic acid (1 mL) was stirred for 30 min. The solvent was removed, the residue taken up in DCM (4 mL) and this solution washed with saturated aqueous Na₂CO₃ (2 mL) and dried (MgSO₄). The solvent was evaporated to afford the title aniline: RT=4.72 min (method 2); m/z (ES⁺)=314.3 [M+H]⁺.

### Example 234

### 4-{3-[3-(2-Hydroxyethylamino)-4-methanesulfonylphenyl]-[1,2,4]oxadiazol-5-ylmethoxy}piperidine-1-carboxylic acid tert-butyl ester

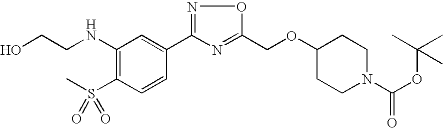

A solution of 4-[3-(3-fluoro-4-methanesulfonylphenyl)-[1,2,4]oxadiazol-5-ylmethoxy]piperidine-1-carboxylic acid tert-butyl ester (Example 134, 6.5 mg, 14 μmol) and ethanolamine (60.5 μL, 1 mmol) in DMF (1 mL) was stirred at rt for 72 h. The solvent was removed and the residue purified by column chromatography (EtOAc then THF) to afford the title compound: RT=3.52 min (method 2); m/z (ES⁺)=497.2 [M+H]⁺.

### Example 235 and 236

### 4-[3-(3-Dimethylamino-4-methanesulfonylphenyl)-[1,2,4]oxadiazol-5-ylmethoxy]piperidine-1-carboxylic acid tert-butyl ester and 4-[3-(3-amino-4-methanesulfonylphenyl)-[1,2,4]oxadiazol-5-ylmethoxy]piperidine-1-carboxylic acid tert-butyl ester

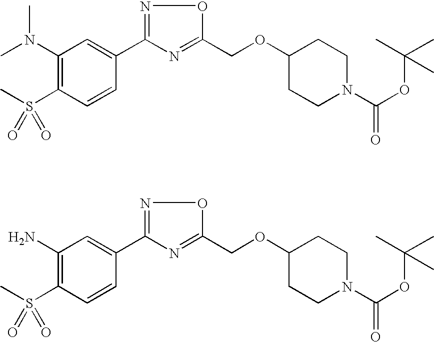

4-[3-(3-Fluoro-4-methanesulfonylphenyl)-[1,2,4]oxadiazol-5-ylmethoxy]piperidine-1-carboxylic acid tert-butyl ester (Example 134, 10 mg, 22 μmol) was dissolved in a solution of 1 M NH₃ in DMF (1 mL) in a sealed tube and heated at 80° C. for 65 h. The solvent was removed and the residue purified by preparative thin-layer chromatography (IH-EtOAc 1:1) to afford 235 as the less-polar component: RT=3.77 min (method 2); m/z (ES⁺)=481.3 [M+H]⁺ and 236: RT=3.59 min; m/z (ES⁺)=453.3 [M+H]⁺.

### Example 237

### 4-[3-(3,4-Dicyanophenyl)-[1,2,4]oxadiazol-5-ylmethoxy]piperidine-1-carboxylic acid tert-butyl ester

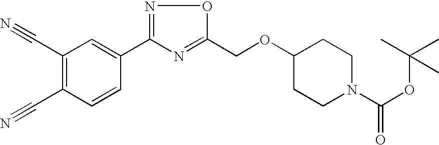

Sodium cyanide (23 mg, 470 μmol) and 4-[3-(3-fluoro-4-methanesulfonylphenyl)-[1,2,4]oxadiazol-5-ylmethoxy]piperidine-1-carboxylic acid tert-butyl ester (Example 134, 10 mg, 22 μmol) were weighed into a small vessel and DMSO (0.5 mL) added. The mixture was stirred for 72 h at rt then diluted with EtOAc (10 mL) and washed with water (2×3 mL) and brine (2 mL). After drying (MgSO₄) the solvent was removed and the residue purified by column chromatography (IH-EtOAc 1:1) to afford the title compound. RT=3.81 min (method 2); m/z (ES⁺)=345.2 [M+H—C₄H₈]⁺.

### Example 238

### 4-[3-(3-Fluoro-4-propionylaminophenyl)-[1,2,4]oxadiazol-5-ylmethoxy]piperidine-1-carboxylic acid tert-butyl ester

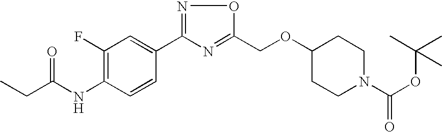

A slurry of 10% Pd on C (87 mg, 82 μmol) in EtOAc (1 mL) was added to a solution of 4-[3-(3-fluoro-4-nitrophenyl)-[1,2,4]oxadiazol-5-ylmethoxy]piperidine-1-carboxylic acid tert-butyl ester Example 65 (700 mg, 1.66 mmol) in EtOAc (30 mL) and the mixture stirred under a hydrogen atmosphere for 18 h. After filtering through a pad of Celite, the solvent was removed to afford 4-[3-(4-amino-3-fluorophenyl)-[1,2,4]oxadiazol-5-ylmethoxy]piperidine-1-carboxylic acid tert-butyl ester: RT=3.65 min (method 2); m/z (ES⁺)=393.1 [M+H]⁺. A sample of this aniline (110 mg, 281 μmol) in anhydrous THF (5 mL) was treated firstly with triethylamine (117 μL, 840 μmol) then propionyl chloride (49 μL, 560 μmol). After stirring for 17 h, the reaction mixture was diluted with EtOAc (80 mL), washed with saturated aqueous NaHCO₃ (10 mL), brine (10 mL) and dried (MgSO₄). Removal of the solvent and purification of the residue by column chromatography (IH-EtOAc 3:2) afforded the title amide RT=3.74 min (method 2); m/z (ES⁺)=449.1 [M+H]⁺.

The biological activity of the compounds of the invention may be tested in the following assay systems:

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast GPCR, Ste3 has been deleted to allow for heterologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (101 g), 21 g of each of two Fus1p-LacZ reporter plasmids (one with URA selection marker and one with TRP), 2 μg of GPR119 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μl of yeast cells added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di(β-D-galactopyranoside) was used (FDG), a substrate for the enzyme that releases fluorescein, allowing a fluorimetric read-out. 20 μl per well of 500 μM FDG/2.5% Triton X100 was added (the detergent was necessary to render the cells permeable). After incubation of the cells with the substrate for 60 min, 20 μl per well of 1M sodium carbonate was added to terminate the reaction and enhance the fluorescent signal. The plates were then read in a fluorimeter at 485/535 nm.

The compounds of the invention give an increase in fluorescent signal of at least ˜1.5-fold that of the background signal (i.e. the signal obtained in the presence of 1% DMSO without compound). Compounds of the invention which give an increase of at least 5-fold may be preferred.

cAMP Assay

A stable cell line expressing recombinant human GPR119 was established and this cell line was used to investigate the effect of compounds of the invention on intracellular levels of cyclic AMP (cAMP). The cell monolayers were washed with phosphate buffered saline and stimulated at 37° C. for 30 min with various concentrations of compound in stimulation buffer plus 1% DMSO. Cells were then lysed and cAMP content determined using the Perkin Elmer AlphaScreen™ (Amplified Luminescent Proximity Homogeneous Assay) cAMP kit Buffers and assay conditions were as described in the manufacturer's protocol.

Compounds of the invention produced a concentration-dependent increase in intracellular cAMP level and generally had an EC₅₀ of <10 μM. Compounds showing an EC₅₀ of less than 1 μM in the cAMP assay may be preferred.

### In Vivo Feeding Study

The effect of compounds of the invention on body weight and food and water intake was examined in freely-feeding male Sprague-Dawley rats maintained on reverse-phase lighting. Test compounds and reference compounds were dosed by appropriate routes of administration (e.g. intraperitoneally or orally) and measurements made over the following 24 h. Rats were individually housed in polypropylene cages with metal grid floors at a temperature of 21±4° C. and 55±20% humidity. Polypropylene trays with cage pads were placed beneath each cage to detect any food spillage. Animals were maintained on a reverse phase light-dark cycle (lights off for 8 h from 09.30-17.30 h) during which time the room was illuminated by red light. Animals had free access to a standard powdered rat diet and tap water during a two week acclimatization period. The diet was contained in glass feeding jars with aluminum lids. Each lid had a 3-4 cm hole in it to allow access to the food. Animals, feeding jars and water bottles were weighed (to the nearest 0.1 g) at the onset of the dark period. The feeding jars and water bottles were subsequently measured 1, 2, 4, 6 and 24 h after animals were dosed with a compound of the invention and any significant differences between the treatment groups at baseline compared to vehicle-treated controls.

Selected compounds of the invention showed a statistically significant hypophagic effect at one or more time points at a dose of ≦100 mg/kg.

### Anti-Diabetic Effects of Compounds of the Invention in an In-Vitro Model of Pancreatic Beta Cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP Assay

HIT-T15 cells were plated in standard culture medium in 96-well plates at 100,000 cells/0.1 ml/well and cultured for 24 hr and the medium was then discarded. Cells were incubated for 15 min at room temperature with 100 μl stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 ul lysis buffer (5 mM HEPES, 0.3% Tween-20, 0.1% BSA, pH 7.4) was added per well and the plate was shaken at 900 rpm for 20 min. Particulate matter was removed by centrifugation at 3000 rpm for 5 min, then the samples were transferred in duplicate to 384-well plates, and processed following the Perkin Elmer AlphaScreen cAMP assay kit instructions. Briefly 25 μl reactions were set up containing 8 μl sample, 5 μl acceptor bead mix and 12 μl detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC₅₀ of less than 10 μM. Compounds showing an EC₅₀ of less than 1 μM in the cAMP assay may be preferred.

### Insulin Secretion Assay

HIT-T15 cells were plated in standard culture medium in 12-well plates at 106 cells/1 ml/well and cultured for 3 days and the medium was then discarded. Cells were washed ×2 with supplemented Krebs-Ringer buffer (KRB) containing 119 mM NaCl, 4.74 mm KCl, 2.54 mM CaCl₂, 1.19 mM MgSO₄, 1.19 mM KH2PO4, 25 mM NaHCO₃, 10 mM HEPES at pH 7.4 and 0.1% bovine serum albumin. Cells were incubated with 1 ml KRB at 37° C. for 30 min which was then discarded. This was followed by a second incubation with KRB for 30 min, which was collected and used to measure basal insulin secretion levels for each well. Compound dilutions (0, 0.1, 0.3, 1, 3, 10 uM) were then added to duplicate wells in 1 ml KRB, supplemented with 5.6 mM glucose. After 30 min incubation at 37° C. samples were removed for determination of insulin levels. Measurement of insulin was done using the Mercodia Rat insulin ELISA kit, following the manufacturers instructions, with a standard curve of known insulin concentrations. For each well insulin levels were corrected by subtraction of the basal secretion level from the pre-incubation in the absence of glucose. Data was analysed using XLfit 3 software.

Representative compounds of the invention were found to increase insulin secretion at an EC₅₀ of less than 10 μM. Compounds showing an EC₅₀ of less than 1 μM in the insulin secretion assay may be preferred.

### Oral Glucose Tolerance Tests

The effects of compounds of the invention on oral glucose (Glc) tolerance were evaluated in male C57B1/6 or male ob/ob mice. Food was withdrawn 5 h before administration of Glc and remained withdrawn throughout the study. Mice had free access to water during the study. A cut was made to the animals' tails, then blood (20 μL) was removed for measurement of basal Glc levels 45 min before administration of the Glc load. Then, the mice were weighed and dosed orally with test compound or vehicle (20% aqueous hydroxypropyl-β-cyclodextrin or 25% aqueous Gelucire 44/14) 30 min before the removal of an additional blood sample (20 μL) and treatment with the Glc load (2-5 g kg⁻¹ p.o.). Blood samples (20 μL) were then taken 25, 50, 80, 120, and 180 min after Glc administration. The 20 μL blood samples for measurement of Glc levels were taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood were then added to 180 μL of Trinders glucose reagent (Sigma enzymatic (Trinder) colorimetric method) in a 96-well assay plate. After mixing, the samples were left at rt for 30 min before being read against Glc standards (Sigma glucose/urea nitrogen combined standard set). Representative compounds of the invention statistically reduced the Glc excursion at doses ≦100 mg kg⁻¹.

What is claimed is:

1. A compound of formula (I), or a pharmaceutically acceptable salt thereof: 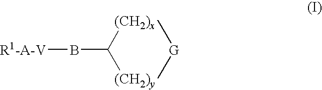
wherein V is a 5-membered heteroaryl ring containing up to four heteroatoms selected from O, N and S, which is optionally substituted by C₁₋₄ alkyl;
A is —CH═CH— or (CH₂)ₙ;
B is —CH═CH— or (CH₂)ₙ, where one of the CH₂ groups may be replaced by O, NR⁵, S(O)ₘ, C(O), C(O)NR⁵, CH(NR⁵R⁵⁵), CH(OH), C(O)O, C(O)S, SC(O) or OC(O);
n is independently 0, 1, 2 or 3;
m is independently 0, 1 or 2;
x is 0, 1, 2 or 3;
y is 1, 2, 3, 4 or 5;
with the proviso that x+y is 2, 3, 4 or 5;
G is CHR¹² or NR²;
R¹ is phenyl or a 5- or 6-membered heteroaryl group containing up to four heteroatoms selected from O, N and S, any of which may be optionally substituted by one or more substituents selected from halo, C₁₋₄ alkyl, C₁₋₄ fluoroalkyl, C₁₋₄ hydroxyalkyl, C₂₋₄ alkenyl, C₂₋₄ alkynyl, C₃₋₇ cycloalkyl, aryl, OR⁶, CN, NO₂, —(CH₂)ⱼ—S(O)ₘR⁶, —(C₂)ⱼ—C(O)NR⁶R⁶⁶, NR⁶R⁶⁶, NR¹⁰C(O)R⁶, NR¹⁰C(O)NR⁶R⁶⁶, NR¹⁰SO₂R⁶, SO₂NR⁶R⁶⁶, C(O)R¹⁰, C(O)OR¹⁰, —(CH₂)ⱼ-(4- to 7-membered heterocyclyl) or —(CH₂)ⱼ-(5- to 6-membered heteroaryl); provided that R¹ is not optionally substituted 3- or 4-pyridyl, 4- or 5-pyrimidinyl or 2-pyrazinyl;
j is 0, 1 or 2;
R² is C(O)OR³, C(O)NR³R¹³, C₁₋₄alkylene-C(O)OR³, C(O)C(O)OR³, S(O)₂R³, C(O)R³ or P(O)(O-Ph)₂; or heterocyclyl or heteroaryl, either of which may optionally be substituted by one or two groups selected from C₁₋₄alkyl, C₁₋₄alkoxy or halogen;
R³ is C₁₋₈ alkyl, C₂₋₈ alkenyl or C₂₋₈ alkynyl, any of which may be optionally substituted by one or more halo atoms, NR⁴R⁴⁴, OR⁴, C(O)OR⁴, OC(O)R⁴ or cyano, and may contain a CH₂ group that is replaced by O or S; or C₃₋₇ cycloalkyl, aryl, heterocyclyl, heteroaryl, C₁₋₄ alkyleneC₃₋₇ cycloalkyl, C₁₋₄ alkylenearyl, C₁₋₄ alkyleneheterocyclyl or C₁₋₄ alkyleneheteroaryl, any of which may be substituted with one or more substituents selected from halo, C₁₋₄ alkyl, C₁₋₄ fluoroalkyl, OR⁴, CN, NR⁴R⁴⁴, SO₂Me, NO₂ or C(O)OR⁴;
R⁴ and R⁴⁴ are independently hydrogen or C₁₋₄alkyl; or, taken together, R⁴ and R⁴⁴ may form a 5- or 6-membered heterocyclic ring;
R⁵ and R⁵⁵ independently represent hydrogen or C₁₋₄ alkyl;
R⁶ and R⁶⁶ are independently hydrogen or C₁₋₄ alkyl, which may optionally be substituted by halo, hydroxy, C₁₋₄ alkyloxy-, C₁₋₄ alkylthio-, C₃₋₇ heterocyclyl, —C(O)OR¹⁴ or N(R¹⁰)₂; or C₃₋₇ cycloalkyl, aryl, heterocyclyl or heteroaryl, wherein the cyclic groups may be substituted with one or more substituents selected from halo, C₁₋₄ alkyl, C₁₋₄ fluoroalkyl, OR⁹, CN, SO₂CH₃, N(R¹⁰)₂ and NO₂; or, taken together, R⁶ and R⁶⁶ may form a 4- to 6-membered heterocyclic ring optionally substituted by hydroxy, C₁₋₄ alkyl or C₁₋₄ hydroxyalkyl and optionally containing a further heteroatom selected from O and NR¹¹, or R⁶⁶ is C₁₋₄ alkyloxy-;
R⁹ is hydrogen, C₁₋₂ alkyl or C₁₋₂ fluoroalkyl;
R¹⁰ are independently hydrogen or C₁₋₄ alkyl; or a group N(R¹⁰)₂ may form a 4- to 7-membered heterocyclic ring optionally containing a further heteroatom selected from O and NR¹⁰;
R¹² is C₃₋₆alkyl; and
R¹³ and R¹⁴ are independently hydrogen or C₁₋₄ alkyl;
provided that the compound is not:
4-(3-phenyl-[1,2,4]oxadiazol-5-yl)piperidine-1-carboxylic acid tert-butyl ester;
4-[3-(4-fluorophenyl)-[1,2,4]oxadiazol-5-yl]piperidine-1-carboxylic acid tert-butyl ester;
4-[3-(4-chlorophenyl)-[1,2,4]oxadiazol-5-yl]piperidine-1-carboxylic acid tert-butyl ester;
4-[3-(4-bromophenyl)-[1,2,4]oxadiazol-5-yl]piperidine-1-carboxylic acid tert-butyl ester;
4-[3-(4-iodophenyl)-[1,2,4]oxadiazol-5-yl]piperidine-1-carboxylic acid tert-butyl ester;
4-[3-(4-nitrophenyl)-[1,2,4]oxadiazol-5-yl]piperidine-1-carboxylic acid tert-butyl ester;
4-[3-(4-methoxyphenyl)-[1,2,4]oxadiazol-5-yl]piperidine-1-carboxylic acid tert-butyl ester;
4-(3-p-tolyl-[1,2,4]oxadiazol-5-yl)piperidine-1-carboxylic acid tert-butyl ester;
4-(3-thiophen-2-yl-[1,2,4]oxadiazol-5-yl)piperidine-1-carboxylic acid tert-butyl ester; or
4-(3-thiophen-2-ylmethyl-[1,2,4]oxadiazol-5-yl)piperidine-1-carboxylic acid tert-butyl ester.

2. A compound according to claim 1, or a pharmaceutically acceptable salt thereof, wherein V represents a 5-membered heteroaryl ring containing up to three heteroatoms selected from O, N and S of the formula: 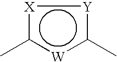
wherein W, X and Y represent the positions of the heteroatom(s) or otherwise represent CH.

3. A compound according to claim 2, or a pharmaceutically acceptable salt thereof, wherein two of W, X and Y are N, and the other is O.

4. A compound according to claim 3, or a pharmaceutically acceptable salt thereof, wherein W is N.

5. A compound according to claim 1, or a pharmaceutically acceptable salt thereof, wherein the n groups of A and B do not both represent 0.

6. A compound according to claim 1, or a pharmaceutically acceptable salt thereof, wherein in A is (CH₂)ₙ wherein n is 0, 1 or 2.

7. A compound according to claim 6, or a pharmaceutically acceptable salt thereof, wherein in A, n is 0.

8. A compound according to claim 1, or a pharmaceutically acceptable salt thereof, wherein B is (CH₂)ₙ wherein, n is 1, 2 or 3.

9. A compound according to claim 8, or a pharmaceutically acceptable salt thereof, wherein in B, n is 2 or 3.

10. A compound according to claim 9, or a pharmaceutically acceptable salt thereof, wherein in B, n is 2.

11. A compound according to claim 1, or a pharmaceutically acceptable salt thereof, wherein R¹ is phenyl or 6-membered heteroaryl group containing up to two heteroatoms selected from O, N and S, either of which may be optionally substituted.

12. A compound according to claim 11, or a pharmaceutically acceptable salt thereof, wherein R¹ is optionally substituted phenyl.

13. A compound according to claim 1, or a pharmaceutically acceptable salt thereof, wherein G is NR².

14. A compound according to claim 1, or a pharmaceutically acceptable salt thereof, wherein x and y each represent 1.

15. A compound according to claim 1 to 13, or a pharmaceutically acceptable salt thereof, wherein x and y each represent 2.

16. A compound according to claim 1, or a pharmaceutically acceptable salt thereof, wherein R² is C(O)OR³, C(O)NR³R¹³ or heteroaryl.

17. A compound according to claim 16, or a pharmaceutically acceptable salt thereof, wherein R² is C(O)OR³.

18. A compound according to claim 1, or a pharmaceutically acceptable salt thereof, wherein R³ represents C₁₋₈ alkyl, C₂₋₈ alkenyl or C₂₋₈ alkynyl optionally substituted by one or more halo atoms or cyano, and which may contain a CH₂ group that may be replaced by O or S; or a C₃₋₇ cycloalkyl or aryl, either of which may be substituted with one or more substituents selected from halo, C₁₋₄ alkyl, C₁₋₄ fluoroalkyl, OR⁴, CN, NR⁴R⁴⁴, NO₂ or C(O)OC₁₋₄alkyl.

19. A compound according to claim 18, or a pharmaceutically acceptable salt thereof, wherein R³ represents C₂₋₅alkyl optionally substituted by one or more halo atoms or cyano, and which may contain a CH₂ group that is replaced by O or S; or C₃₋₅ cycloalkyl optionally substituted by halo, C₁₋₄ alkyl, C₁₋₄ fluoroalkyl, OR⁴, CN, NR⁴R⁴⁴, NO₂ or C(O)OC₁₋₄alkyl.

20. A compound according to claim 1, or a pharmaceutically acceptable salt thereof, of formula (Ib): 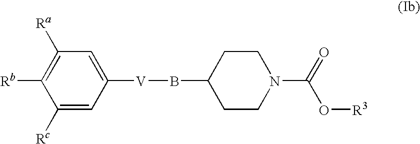
wherein:
R^{a} and R^{c} independently represent hydrogen, fluorine, chlorine, methyl or CN;
R^{b} represents S(O)ₘR⁶, C(O)NR⁶R⁶⁶SO₂NR⁶R⁶⁶, NR¹⁰C(O)R⁶, NR¹⁰SO₂R⁶, NR¹⁰C(O)NR⁶R⁶⁶ or 5-membered heteroaryl;
R³ represents C₂₋₅ alkyl or C₃₋₅ cycloalkyl which may optionally be substituted by methyl;
m represents 1 or 2;
R⁶ and R⁶⁶ independently represent hydrogen or C₁₋₄ alkyl which may optionally be substituted by hydroxyl or NH₂, alternatively R⁶ and R⁶⁶ taken together may form a 5- or 6-membered heterocyclic ring optionally substituted with OH or CH₂OH; and
R¹⁰ are independently hydrogen or C₁₋₄ alkyl; or a group N(R¹⁰)₂ may form a 4- to 7-membered heterocyclic ring optionally containing a further heteroatom selected from O and NR¹⁰.

21. A compound of formula (I) as defined in any one of Examples 1 to 238, or a pharmaceutically acceptable salt thereof.

22. A pharmaceutical composition comprising a compound according to claim 1, or a pharmaceutically acceptable salt thereof; and a pharmaceutically acceptable carrier.

23. A method for the treatment of a disease or condition in which GPR119 plays a role comprising a step of administering to a subject in need thereof an effective amount of a compound according to claim 1, or a pharmaceutically acceptable salt thereof.

24. A method for the regulation of satiety comprising a step of administering to a subject in need thereof an effective amount of a compound according to claim 1, or a pharmaceutically acceptable salt thereof.

25. A method for the treatment of obesity comprising a step of administering to a subject in need thereof an effective amount of a compound according to claim 1, or a pharmaceutically acceptable salt thereof.

26. A method for the treatment of diabetes comprising a step of administering to a subject in need thereof an effective amount of a compound according to claim 1, or a pharmaceutically acceptable salt thereof.

27. A method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound according to claim 1, or a pharmaceutically acceptable salt thereof.

28. (canceled)

29. (canceled)

30. (canceled)

31. A compound of formula (XXVII): 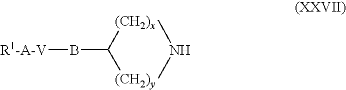
or a salt or protected derivative thereof, wherein the groups R¹, A, V, B, x and y are as defined in claim 1;
provided that the compound is not:
4-(3-phenyl-[1,2,4]oxadiazol-5-yl)piperidine;
4-[3-(4-fluorophenyl)-[1,2,4]oxadiazol-5-yl]piperidine;
4-[3-(4-chlorophenyl)-[1,2,4]oxadiazol-5-yl]piperidine;
4-[3-(4-bromophenyl)-[1,2,4]oxadiazol-5-yl]piperidine;
4-[3-(4-iodophenyl)-[1,2,4]oxadiazol-5-yl]piperidine;
4-[3-(4-nitrophenyl)-[1,2,4]oxadiazol-5-yl]piperidine;
4-[3-(4-methoxyphenyl)-[1,2,4]oxadiazol-5-yl]piperidine;
4-(3-p-tolyl-[1,2,4]oxadiazol-5-yl)piperidine;
4-(3-thiophen-2-yl-[1,2,4]oxadiazol-5-yl)piperidine;
4-(3-thiophen-2-ylmethyl-[1,2,4]oxadiazol-5-yl)piperidine;
4-[5-(4-tert-butylphenyl)-[1,2,4]oxadiazol-3-ylmethyl]piperidine;
4-[5-(biphen-4-yl)-[1,2,4]oxadiazol-3-yl]piperidine; or
4-[3-(biphen-4-yl)-[1,2,4]oxadiazol-5-yl]piperidine.
